(19) United States
(12) Patent Application Publication
(10) Pub. No.: US 2022/0370471 A1
(43) Pub. Date: Nov. 24, 2022

(54) COMPOSITION AND METHOD FOR TREATMENT OF HIPPOCAMPAL SYNAPSE DYSFUNCTION AND COGNITIVE DEFICITS IN ALZHEIMER'S DISEASE

(71) Applicant: BOARD OF REGENTS, THE UNIVERSITY OF TEXAS SYSTEM, Austin, TX (US)

(72) Inventor: Heng Du, Richardson, TX (US)

(73) Assignee: BOARD OF REGENTS, THE UNIVERSITY OF TEXAS SYSTEM, Austin, TX (US)

(21) Appl. No.: 17/634,850

(22) PCT Filed: Aug. 13, 2020
(86) PCT No.: PCT/US2020/046062

§ 371 Date: Feb. 11, 2022

Related U.S. Application Data
(60) Provisional application No. 62/886,079, filed on Aug. 13, 2019.
(60) Provisional application No. 62/986,440, filed on Mar. 6, 2020.

(51) Int. Cl. A61K 31/55; A61K 31/438; A61K 45/06; A61P 25/28

(52) U.S. Cl. CPC A61K 31/55; A61K 31/438; A61K 45/06; A61P 25/28

(57) ABSTRACT

Hippocampal lesions, including synaptic failure, are a defining pathology of Alzheimer's disease (AD). However, the molecular mechanisms that underlie hippocampal synaptic injury in this neurodegenerative disorder have yet not been fully elucidated. In Current therapeutic efforts for the treatment of AD are not effective in correcting hippocampal synaptic deficits. It is disclosed that the interaction with amyloid beta (Aβ) induces dysfunction of growth hormone secretagogue receptor 1a (GHSR1α; ghrelin receptor), leading to suppressed GHSR1α regulation of hippocampal dopamine receptor D1 (DRD1) in AD hippocampi. The modulation of DRD1 by GHSR1α impacts hippocampal synaptic plasticity. The simultaneous application of a selective GHSR1α agonist and a selective DRD1 agonist mitigates Aβ-induced hippocampal synaptic injury and improves spatial memory in AD mouse models. These data reveal a novel mechanism of hippocampal vulnerability in AD and that the combined activation of GHSR1α and DRD1 provides a new avenue for the pharmaceutical treatment of AD.

### PRIORITY INFORMATION

This application is a national phase application under 35 U.S.C. § 371 of International Application No. PCT/US2020/046062, filed Aug. 13, 2020, which claims benefit of priority to U.S. Provisional Application Ser. Nos. 62/886,079, and 62/986,440, filed Aug. 13, 2019 and Mar. 6, 2020, respectively, the entire contents of each application being hereby incorporated by reference.

### STATEMENT REGARDING FEDERALLY-SPONSORED RESEARCH OR DEVELOPMENT

This invention was made with government support under Grant Nos. R00 AG037716, R01 AG053588 and R01 AG059753 awarded by the National Institutes of Health. The government has certain rights in the invention.

### STATEMENT REGARDING OTHER FUNDING

This work was supported by a grant from the Alzheimer's Association (AARG-16-442863).

### BACKGROUND

### 1. Field

This invention relates to a novel therapeutic strategy for the treatment of synaptic failure and cognitive deficits in AD by targeting to GHSR dysfunction.

### 2. Description of the Related Art

Alzheimer's disease (AD) is a devastating neurodegenerative disorder in which cognitive impairments and amyloid beta (Aβ) toxicity converge¹. Clinical observations have shown that hippocampal lesions are an early and defining AD pathology that underlies the decline of cognitive activity²⁻⁴. Currently no effective therapy exists that corrects hippocampal synaptic deficits in AD⁵, which underscores the need to elucidate the precise mechanisms through which Aβ toxicity contributes to the selective hippocampal vulnerability. Growth hormone secretagogue receptor 1a (GHSR1α), also known as ghrelin receptor, is a member of the class A G-protein coupled receptor (GPCR) family. In addition to its abundance in the pituitary gland and hypothalamus, GHSR1α is highly expressed in the hippocampus including both the dentate gyms and Ammon's horn⁶⁻⁸, indicating its relevance to hippocampal function. Indeed, several studies have demonstrated specific roles for hippocampal ghrelin signaling in more complex forms of eating, such as those with learning, motivational, and hedonic components⁸⁻¹⁰.

Emerging evidence suggests that GHSR1α is critical for hippocampal synaptic physiology through its downstream signaling and specifically its regulation of the dopamine receptor D1 (DRD1)¹¹⁻¹⁵. Importantly, the modulation of DRD1 signaling by GHSR1α is critical for initiating hippocampal synaptic plasticity and transmission¹¹. This pivotal role of GHSR1α in hippocampal synaptic function raises the question whether GHSR1α dysfunction contributes to hippocampal synaptic deficits in AD. However, previous studies revealed inconsistent effects of GHSR1α activation on AD phenotypes in patients¹⁶⁻¹⁸ , as well as in AD animal and cell models¹⁹⁻²².

The inventor's own recent studies in patients with mild cognitive impairments (MCI) due to AD showed an unexpected negative correlation between cognitive performance and the levels of the acylated form of ghrelin, which is the endogenous GHSR1α ligand²³. These results implicate that in AD, hippocampal GHSR1α becomes insensitive to activation by agonists or their endogenous ligand.

Previous studies into the function of GHSR1α focused predominantly on its role in the regulation of feeding behavior^{8,9}. Recent studies have highlighted the importance of GHSR1α for hippocampal synaptic plasticityl^{11,13,14}. The selective abundance of GHSR1α in the hippocampus and its role in hippocampal synaptic physiology prompted the inventor's interest in GHSR1α and hippocampal synaptic injury in AD. But information on GHSR1α and the ghrelin system in the pathogenesis of AD is limited. There is a need to better understand the functional status of hippocampal GHSR1α in AD-related conditions to provide insights into the molecular mechanisms of hippocampal pathology and to find a novel target for therapeutic interventions.

### REFERENCES FOR BACKGROUND

1. Querfurth, H. W. & LaFerla, F. M. Alzheimer's disease. N Engl J Med 362, 329-344 (2010).
2. Scheff, S. W., Price, D. A., Schmitt, F. A. & Mufson, E. J. Hippocampal synaptic loss in early Alzheimer's disease and mild cognitive impairment. Neurobiol Aging 27, 1372-1384 (2006).
3. Tampellini, D. Synaptic activity and Alzheimer's disease: a critical update. Front Neurosci 9, 423 (2015).
4. Frisoni, G. B., et al. Mapping local hippocampal changes in Alzheimer's disease and normal ageing with MRI at 3 Tesla. Brain 131, 3266-3276 (2008).
5. Graham, W. V., Bonito-Oliva, A. & Sakmar, T. P. Update on Alzheimer's Disease Therapy and Prevention Strategies. Annu Rev Med 68, 413-430 (2017).
6. Zigman, J. M., Jones, J. E., Lee, C. E., Saper, C. B. & Elmquist, J. K. Expression of ghrelin receptor mRNA in the rat and the mouse brain. J Comp Neurol 494, 528-548 (2006).
7. Mani, B. K., et al. Neuroanatomical characterization of a growth hormone secretagogue receptor- green fluorescent protein reporter mouse. J Comp Neurol 522, 3644-3666 (2014).
8. Hsu, T. M., et al. Hippocampus ghrelin signaling mediates appetite through lateral hypothalamic orexin pathways. eLife 4(2015).
9. Kanoski, S. E., Fortin, S. M., Ricks, K. M. & Grill, H. J. Ghrelin signaling in the ventral hippocampus stimulates learned and motivational aspects of feeding via PI3K-Akt signaling. Biol Psychiatry 73, 915-923 (2013).
10. Hsu, T. M., et al. Hippocampus ghrelin receptor signaling promotes socially-mediated learned food preference. Neuropharmacology 131, 487-496 (2018).
11. Kern, A., et al. Hippocampal Dopamine/DRD1 Signaling Dependent on the Ghrelin Receptor. Cell 163, 1176-1190 (2015).
12. Kim, C., Kim, S. & Park, S. Neurogenic Effects of Ghrelin on the Hippocampus. International journal of molecular sciences 18(2017).
13. Diano, S., et al. Ghrelin controls hippocampal spine synapse density and memory performance. Nat Neurosci 9, 381-388 (2006).
14. Ribeiro, L. F., et al. Ghrelin triggers the synaptic incorporation of AMPA receptors in the hippocampus. Proc Natl Acad Sci USA 111, E149-158 (2014).
15. Kern, A., Grande, C. & Smith, R. G. Apo-Ghrelin Receptor (apo-GHSR1α) Regulates Dopamine Signaling in the Brain. Frontiers in endocrinology 5, 129 (2014).
16. Sevigny, J. J., et al. Growth hormone secretagogue MK-677: no clinical effect on AD progression in a randomized trial. Neurology 71, 1702-1708 (2008).
17. Gahete, M. D., Cordoba-Chacon, J., Kineman, R. D., Luque, R. M. & Castano, J. P. Role of ghrelin system in neuroprotection and cognitive functions: implications in Alzheimer's disease. Peptides 32, 2225-2228 (2011).
18. Dos Santos, V. V., et al. Ghrelin as a neuroprotective and palliative agent in Alzheimer's and Parkinson's disease. Curr Pharm Des 19, 6773-6790 (2013).
19. Kunath, N., et al. Ghrelin agonist does not foster insulin resistance but improves cognition in an Alzheimer's disease mouse model. Sci Rep 5, 11452 (2015).
20. Cecarini, V., et al. Effects of Ghrelin on the Proteolytic Pathways of Alzheimer's Disease Neuronal Cells. Molecular neurobiology 53, 3168-3178 (2016).
21. Moon, M., Cha, M. Y. & Mook-Jung, I. Impaired hippocampal neurogenesis and its enhancement with ghrelin in SXFAD mice. J Alzheimers Dis 41, 233-241 (2014).
22. Santos, V. V., et al. Acyl ghrelin improves cognition, synaptic plasticity deficits and neuroinflammation following amyloid beta (Abetal-40) administration in mice. J Neuroendocrinol 29(2017).

### SUMMARY

Disclosed is a drug composition or drug compositions and method providing for combined activation of GHSR1α and DRD1 with the selective agonists for rescuing hippocampal synaptic function and cognition in AD patients. Increased GHSR1α expression and a direct interaction of GHSR1α with Aβ in the hippocampi from AD cases is found. The interaction with Aβ inhibits GHSR1α activation and prevents GHSR1α/DRD1 heteromerization. The functional loss of GHSR1α replicates hippocampal synaptic stress and cognitive impairments seen in AD mouse models. In particular, the combined activation of GHSR1α and DRD1 with the selective agonists MK0677 and SKF81297, respectively, rescues hippocampal synaptic function and cognition.

Thus, in accordance with the disclosure, there is provided a method for treatment of hippocampal synaptic dysfunction and Alzheimer's disease in a patient comprising the step of administering one more drug compositions to the patient wherein the drug composition is configured to promote the heteromerization of GHSR1α and DRD1. The method of applying the one or more drug compositions may include the step of simultaneously administering an agonist of GHSR1α and an agonist of DRD1 to the patient, such as where both agonists are co-formulated into a single composition, or where the drugs are administered as separate drug compositions. The method may further comprise the step of selecting the agonist of GHSR1α from the group consisting of: Adenosine, Alexamorelin, Anamorelin, Capromorelin, CP-464709, Cortistatin-14, Examorelin (hexarelin), Ghrelin (lenomorelin), GHRP-1, GHRP-3, GHRP-4, GHRP-5, GHRP-6, Ibutamoren (MK-0677), Ipamorelin, L-692,585, LY-426410, LY-444711, Macimorelin, Pralmorelin, (GHRP-2), Relamorelin, SM-130,686, Tabimorelin and Ulimorelin. The method may further comprise the step of selecting the agonist of DRD1 from the group consisting of: dopamine, A-86929, Dihydrexidine, Dinapsoline, Dinoxyline, Doxanthrine, SKF-81297, SKF-82958, SKF-38393, Fenoldopam, 6-Br-APB, Stepholidine, A-68930, A-77636, CY-208,243, SKF-89145, SKF-89626, 7,8-Dihydroxy-5-phenyl-octahydrobenzo[h]isoquinoline, Cabergoline, Pergolide, levodopa and carbidopa. The agonist of GHSR1α may be MK0677 and the agonist of DRD1 may be SKF81297. MK0677 may be combined with SKF81297 in a ratio of about 1:1.5. The agonist of DRD1 may be selected based on 200-fold selectivity for D1.

In another embodiment, there is provided a method for treatment of hippocampal synaptic dysfunction and Alzheimer's disease in a patient comprising the step of simultaneously applying a first drug and a second drug to promote the heteromerization of GHSR1α and DRD1, wherein the first drug is an agonist of GHSR1α and the second drug is an agonist of DRD1. The step of simultaneously applying a first drug and a second drug may further comprise the step of combining the first drug and the second drug into a fixed-dose combination (FDC) product in a single dosage form. The method may further comprise the step of selecting the first drug from the group consisting of: Adenosine, Alexamorelin, Anamorelin, Capromorelin, CP-464709, Cortistatin-14, Examorelin (hexarelin), Ghrelin (lenomorelin), GHRP-1, GHRP-3, GHRP-4, GHRP-5, GHRP-6, Ibutamoren (MK-0677), Ipamorelin, L-692,585, LY-426410, LY-444711, Macimorelin, Pralmorelin, (GHRP-2), Relamorelin, SM-130,686, Tabimorelin and Ulimorelin. The method may further comprise the step of selecting the second drug from the group consisting of: dopamine, A-86929, Dihydrexidine, Dinapsoline, Dinoxyline, Doxanthrine, SKF-81297, SKF-82958, SKF-38393, Fenoldopam, 6-Br-APB, Stepholidine, A-68930, A-77636, CY-208,243, SKF-89145, SKF-89626, 7,8-Dihydroxy-5-phenyl-octahydrobenzo[h]isoquinoline, Cabergoline, Pergolide, levodopa and carbidopa. The first drug may be MK0677 and the second drug may be SKF81297.

In yet another embodiment, there is provided a drug composition for rescuing hippocampal synaptic function in a patient and for rescuing cognition in Alzheimer's disease patients comprising an agonist of GHSR1 and an agonist of DRD1. The ratio of the agonist of GHSR1α to the agonist of DRD1 may be configured to promote the heteromerization of GHSR1α and DRD1. The agonist of GHSR1 and the agonist of DRD1 may be combined into a fixed-dose combination (FDC) product in a single dosage form. The agonist of GHSR1α may be selected from the group consisting of: Adenosine, Alexamorelin, Anamorelin, Capromorelin, CP-464709, Cortistatin-14, Examorelin (hexarelin), Ghrelin (lenomorelin), GHRP-1, GHRP-3, GHRP-4, GHRP-5, GHRP-6, Ibutamoren (MK-0677), Ipamorelin, L-692,585, LY-426410, LY-444711, Macimorelin, Pralmorelin, (GHRP-2), Relamorelin, SM-130,686, Tabimorelin and Ulimorelin. The agonist of DRD1 may be selected from the group consisting of: dopamine, A-86929, Dihydrexidine, Dinapsoline, Dinoxyline, Doxanthrine, SKF-81297, SKF-82958, SKF-38393, Fenoldopam, 6-Br-APB, Stepholidine, A-68930, A-77636, CY-208,243, SKF-89145, S KF- 89626, 7,8-Dihydroxy-5-phenyl-octahydrobenzo [h] isoquinoline, Cabergoline, Pergolide, levodopa and carbidopa. The agonist of GHSR1α may be MK0677 and the agonist of DRD1 may be SKF81297. MK0677 may be combined with SKF81297 in a ratio of about 1:1.5.

Also provide is a fixed dose combination drug for rescuing hippocampal synaptic function in a patient and for rescuing cognition in Alzheimer's disease patients comprising an agonist of GHSR1 and an agonist of DRD1 wherein the ratio of the agonist of GHSR1α to the agonist of DRD1 is configured to promote the heteromerization of GHSR1α and DRD1.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### DESCRIPTION OF THE DRAWINGS

The illustrative embodiments, further objectives and features thereof, will best be understood by reference to the following detailed description of an illustrative embodiment of the present disclosure when read in conjunction with the accompanying drawings.

FIGS. 1a-j show that Aβ physically interacts with GHSR1α. (FIG. 1a) Measurement of PLA-positive dots for Aβ/GHSR1α complex in hippocampi from subjects with AD. Unpaired Student's t-test. n=4 healthy donors or subjects with AD. *** P<0.001. (FIG. 1b) Representative images of quantification in A. Scale bar=100 μm. Arrows indicate Aβ/GHSR1α PLA-positive dots. (FIG. 1c) Analysis of Aβ/Ghsr1α PLA-positive dots in the hippocampal region from 4 and 9 month-old 5×FAD mice. Unpaired Student's t-test. ** P<0.01. n=4 mice per group. (FIG. 1d) Representative 3D-reconstructed images. The slices from 9 month-old Ghsr null mice were used as negative control. Scale bar=10 μm. (FIGS. 1e-g) Analysis of Ghsr1α/Aβ PLA-positive dots in HEK 293T cells expressing different forms of Ghsr1α treated with vehicle or 5 μM oligomeric Aβ42 for 24 hours. Anti-FLAG antibody was used to detect Ghsr1α and its mutants. Unpaired Student's t-test. †P<0.001 vs. cells expressing full-length Ghsr1α without oligomeric Aβ42 treatment. #P<0.001 vs cells expressing Ghsr1α Δaa42-116 with oligomeric AΔ42 treatment. n=4-7. (FIG. 1f) Representative 3D-reconstructed images of Ghsr1α/Aβ PLA-positive dots in HEK 293T cells expressing different forms of Ghsr1α treated with vehicle or oligomeric Aβ42 (upper panels), scale bar=5 μm; and (FIG. 1g) representative 3D-reconstructed images of immunofluorescent staining of different forms of Ghsr1α (lower panels) recognized by anti-FLAG antibody, scale bar=10 μm. (FIG. 1h) Densitometry of all immunoreactive bands generated from Co-IP on HEK 293T cells expressing different forms of Ghsr1α treated with vehicle or 5 μM oligomeric Aβ42 for 24 hours. One-way ANOVA followed by Bonferroni post hoc analysis. *** P<0.001. Data were collected from three independent experiments. n (from left to right)=3, 5, 2, 3, 3. Nonimmune IgG to replace specific FLAG antibody was used for examining specificity of Co-IP. (FIG. 1i) Representative immunoblots showing the interaction of oligomeric Aβ42 with Ghsr1α and Ghsr1α Δaa42-116. (FIG. 1j) Representative immunoblots showing the input of Ghsr1α and Ghsr1α Δaa42-116.

FIGS. 2a-i show the interaction with Aβ disrupts GHSR1α activity. (FIGS. 2a-c) The impact of oligomeric Aβ42 (2 μM, 5 minutes' pretreatment) on Ghsr1α FlAsH-FRET response in the presence or absence of MK0677 (50 μM). (FIG. 2a) FRET ratio quantified from data collected from a microplate reader. The effect of Ghsr1α antagonist JMV2959 (50 μM) against MK0677-induced Ghsr1α activation was used as positive control. Two-way ANOVA followed by Bonferroni post hoc analysis. ** P<0.01, compared with other groups. n=12 per group. (FIG. 2b) Representative confocal microscopy images for FRET pseudocolor ratio (FlAsH^{exECFP/emFlAsH}/ECFP^{exECFP/emECFP}), (top panel), FlAsH^{exECFP/emFlAsH} (middle panel, green color) and ECFP^{exECFP/emECFP} (bottom panel, red color). Scale bar=20 μm. (FIG. 2c) Representative 3D-reconstructed images for Ghsr1α and Drd1 expression in Ghsr1α/Drd1 co-expressing HEK 293T cells. Scale bar=10 μm. (FIG. 2d) Analysis of Ghsr1α/Drd1 PLA-positive dot intensity in Ghsr1α/Drd1 co-expressing HEK 293T cells. Unpaired Student's t-test. Data were collected from 3 independent experiments. *** P<0.001. n=78 cells for vehicle treated group; n=60 cells for the group with oligomeric Aβ42 treatment (5 μM, 24 hours). Anti-Ghsr1α and anti-Drd1 antibodies were used in this experiment. (FIG. 2e) Representative 3D-reconstructed images for Ghsr1α/Drd1 PLA-positive dots in Ghsr1α/Drd1 co-expressing HEK 293T cells. Scale bar=5 μm. (FIG. 2f) Analysis of GHSR1α/DRD1 PLA positive dots in hippocampal sections from patients with AD and healthy controls. Unpaired Student's t-test. *** P<0.001. n=5 per group. (FIG. 2g) Representative images of GHSR1α/DRD1 PLA dots. Scale bar=100 μm. Arrows indicate GHSR1α/DRD1 PLA-positive dots. (FIG. 2h) Analysis of Ghsr1α/Drd1 PLA positive dots in hippocampal CA1 region in 4 and 9 months old 5×FAD mice. Unpaired Student's t-test. * P<0.05, ** P<0.01. n=3 for each group. (FIG. 2i) Representative 3D-reconstructed images of Ghsr1α/Drd1 PLA-positive dots in the hippocampus of 4 and 9 months old non-Tg and 5×FAD mice. Ghsr null mice at 9 months old were used as critical negative control. Scale bar=10 μm.

FIGS. 3a-i show that loss of Ghsr replicates AD-like phenotypes. (FIG. 3a) Analysis of synaptic density in CA1 regions from 4 and 9 month-old mice. One-way ANOVA followed by Bonferroni post hoc analysis, ** P<0.001, *** P<0.001 non-Tg vs. other groups at the same age. 4 month-old mice, non-Tg, n=4; 5×FAD, n=7; Ghsr null mice, n=5; GHSR-Null/5×FAD, n=4. 9 month-old mice, non-Tg, n=4; 5×FAD, n=4; Ghsr null mice, n=4; Ghsr null/5×FAD, n=3. (FIG. 3b) Representative 3D-reconstructed images of synapse staining. vGLUT1 (blue) and PSD95 (red) were used to visualize pre- and post-synaptic terminals, respectively. The overlapped staining of vGLUT1 and PSD95 indicates synapses. Scale bar=20 μm. (FIG. 3c) Time course of LTP and representative fEPSP responses during the baseline period (black trace) and 30 seconds after theta burst simulation (red trace) in 4 groups of mice at 9 months old. One-way ANOVA followed by Bonferroni post hoc analysis, * P<0.05, *** P<0.001 non-Tg vs. other groups. non-Tg, n=5; 5×FAD, n=4; Ghsr null n=5; Ghsr null/5×FAD, n=5. (FIGS. 3d-i) Spatial navigation of four groups of mice in the Morris water maze test. (FIGS. 3d&g) Spatial learning of four groups of mice at 4 (FIG. 3d) and 9 (FIG. 3g) months old. One-way ANOVA followed by Bonferroni post hoc analysis, * P<0.05, ** P<0.01, *** P<0.001, non-Tg vs. other groups on the same day. (FIGS. 3e, 3h) Spatial reference memory of different groups of mice at 4 (FIG. 3e) and 9 (FIG. 3h) months of age. One-way ANOVA followed by Bonferroni post hoc analysis. * P<0.05. (FIGS. 3f, 3i) Swimming speed of four groups of mice at 4 (FIG. 3f) and 9 (FIG. 3i) months old. NS, not significant. 4 month-old non-Tg, n=7; 5×FAD, n=9; Ghsr null mice, n=10; Ghsr null/5×FAD, n=6. 9 month-old non-Tg, n=8; 5×FAD, n=11; Ghsr null mice, n=5; Ghsr null/5×FAD, n=8. NS, not significant.

FIGS. 4a-h show that combined Ghsr1α/Drd1 activation rescues hippocampal synapse in vitro. (FIG. 4a) Effect of different treatments (1.5 μM MK0677, 2 μM SKF81297, or in combination) on synaptic density in hippocampal neurons in the presence or absence of oligomeric Aβ42 (1 μM, 24 hours). Two-way ANOVA followed by Bonferroni post hoc analysis, *** P<0.001 vehicle-treated vs. oligomeric Aβ42-treated groups. Data were collected from 3 independent experiments. n=30-48 neurites. (FIG. 4b) Representative 3D-reconstructed images of synapse staining. vGLUT1 (blue) and PSD95 (red) were used to visualize pre- and post-synaptic terminals, respectively. The dendrites were stained with MAP2 (green). The overlaid staining of vGLUT1/PSD95 identifies synapses. Scale bar=10 μm. (FIG. 4c) Effect of different treatments (1.5 μM MK0677, 2μM SKF81297, or in combination) on Ghsr1α/Drd1 complex in hippocampal neurons in the presence or absence of oligomeric Aβ42 (1 μM, 24 hours). Two-way ANOVA followed by Bonferroni post hoc analysis, ** P<0.01, *** P<0.001 vehicle-treated vs. oligomeric Aβ42-treated groups. Data were collected from 3 independent experiments. n=8-10 neurons. (FIG. 4d) Representative images of Ghsr1α/Drd1 PLA dots. Scale bar=30 μm. (FIG. 4e) Time course of LTP and representative fEPSP responses during the baseline period (black trace) and 30 seconds after theta burst simulation (red trace) in 5 treatment groups at 4 months of age. One-way ANOVA followed by Bonferroni post hoc analysis, ** P<0.01 5×FAD MK0677/SKF81297 vs. 5×FAD Saline. non-Tg saline, n=9; 5×FAD saline, n=10; 5×FAD MK0677/SKF81297, n=7; 5×FAD MK0677, n=9; 5×FAD SKF81297, n=9. (FIGS. 4f-h) mEPSC frequency (FIG. 4f) and amplitude (FIG. 4g) in the indicated groups of 4 month-old mice. One-way ANOVA followed by Bonferroni post hoc analysis, * P<0.05, ** P<0.01. n=6. (FIG. 4h) Representative traces of mEPSC recordings.

FIGS. 5a-c show that co-activation of Ghsr1α and Drd1 preserves Ghsr1α activity from Aβ toxicity. (FIG. 5a) Effect of SKF81297 (100 μM) and MK0677 (50 μM) alone or in combination on Ghsr1α FlAsH-FRET response in the presence or absence of oligomeric Aβ42 (2 μM, 5 minutes' pretreatment). Cells expressing Ghsr1α^{FlAsH/ECFP} alone or co-expressed with Drd1 were used. Data were collected from a microplate reader. Two-way ANOVA followed by Bonferroni post hoc analysis. Data were collected from 3 independent experiments. *** P<0.001. n=9-25 samples. (FIGS. 5b-c) Effect of different treatments (1.5 μM MK0677, 2 μM SKF81297, or in combination) on Aβ/Ghsr1α complex in oligomeric Aβ42 (1 μM, 24 hours)-treated hippocampal neurons. One-way ANOVA followed by Bonferroni post hoc analysis. Data were collected from 3 independent experiments. * P<0.05. n=10 neurons per group. Scale bar=30 μm.

FIGS. 6a-i show that combined Ghsr1α/Drd1 activation protects hippocampal synapse and cognition in vivo. (FIGS. 6a-c) Spatial navigation analysis in four groups of mice treated with vehicle (saline) or MK0677/SKF81297 (MK0677 1 mg/kg; SKF81297 1.5 mg/kg) performing the Morris water maze test. (FIG. 6a) Spatial learning. Two-way ANOVA followed by Bonferroni post hoc analysis. ** P<0.01, *** P<0.001 5×FAD saline vs. other groups. (FIG. 6b) Spatial reference memory. Two-way ANOVA followed by Bonferroni post hoc analysis. *** P<0.001 5×FAD saline vs. other groups. (FIG. 6c) Swimming speed. NS, not significant. non-Tg saline, n=8; 5×FAD saline, n=8; non-Tg MK0677/SKF81297, n=7; 5×FAD MK0677/SKF81297, n=5. (FIGS. 6d-e) Analysis of synaptic density in the hippocampal CA1 region. Two-way ANOVA followed by Bonferroni post hoc analysis. ** P<0.01, *** P<0.001 5×FAD saline vs. other groups. non-Tg saline, n=3; 5×FAD saline, n=4; non-Tg MK0677/SKF81297, n=4; 5×FAD MK0677/SKF81297, n=4. (FIG. 6e) Representative 3D-reconstructed images of synapse staining in the CA1 region. vGLUT1 (blue) and PSD95 (red) were used to visualize pre- and post-synaptic components, respectively. The overlaid staining of vGLUT1 and PSD95 indicates synapses. Scale bar=20 μm. (FIGS. 6f-g) Analysis of Ghsr1α/Drd1 complex in the CA1 region. Two-way ANOVA followed by Bonferroni post hoc analysis. *** P<0.001 5×FAD saline vs. other groups. n=4 per group. (FIG. 6g) Representative 3D-reconstructed images of Ghsr1α/Drd1 PLA-positive dots (red). Nuclei were stained with DAPI. Scale bar=10 μm. (FIG. 6h-i) Analysis of Aβ/Ghsr1α PLA-positive dots in CA1 region. Unpaired Student's t-test. *** P<0.001. n=4 per group. (FIG. 6i) Representative 3D-reconstructed images of Aβ/Ghsr1α PLA dots (red). Nuclei were stained with DAPI. Scale bar=10 μm.

FIGS. 7a-f show that hippocampal amyloidosis and tau phosphorylation remain unaltered in treated 5×FAD mice. (FIG. 7a) Analysis of APP expression level in the hippocampus by using immunoblotting. Unpaired Student's t-test. n=4 per group. NS, not significant. The right panel shows representative images of immunoblotting. (3-actin was used as the loading control. (FIG. 7b) Aβ deposition in the hippocampal region was measured and analyzed by immunostaining using Aβ antibody. Unpaired Student's t-test. NS, not significant. 5×FAD saline mice, n=6; 5×FAD MK0677/SKF81297 mice, n=5. The right panel shows representative images of Aβ staining (red color). The neurons were identified by the staining of NeuN (green color). Scale bar=1 mm. (FIG. 7c) Soluble Aβ40 and 42 amounts in hippocampal homogenate were detected by ELISA assay. Unpaired Student's t-test. NS, not significant. 5×FAD saline mice, n=6; 5×FAD MK0677/SKF81297 mice, n=5. (FIG. 7d) Analysis of intracellular Aβ in hippocampal CA1 neurons. Unpaired Student's t-test. NS, not significant. 5×FAD saline mice, n=8; 5×FAD MK0677/SKF81297 mice, n=5. The right panel shows representative images. Aβ was recognized by anti-Aβ antibody (red color). Neurons were labeled by anti-β-III-tubulin (green color). Nuclei were identified by the staining of DAPI (blue color). The overlaid staining of Aβ and β-III-tubulin indicates intraneuronal Aβ. Scale bar=20 μm. (FIG. 7e) Congo Red staining was used to label extracellular parenchymal Aβ plaques. Unpaired Student's t-test. NS, not significant. 5×FAD saline mice, n=8; 5×FAD MK0677/SKF81297 mice, n=5. The right panel shows representative images of Aβ plaque staining. Scale bar=1 mm. (FIG. 7f) Immunoblotting analysis of Tau phosphorylation at different motifs and total Tau in mouse hippocampal tissues. Unpaired Student's t-test. NS, not significant. n=4 per group. The lower panel shows representative images of immunoblotting. β-actin was used as the loading control. P-Tau stands for phosphorylated Tau and T-Tau stands for total Tau.

FIG. 8 shows an embodiment of a method of treatment for hippocampal dysfunction and/or Alzehimer's disease.

FIGS. 9a-b show combined Ghsr1α agonists/SKF81297 treatments rescue hippocampal synapse in vitro. (FIG. 9a) Effect of different treatments (1 μM ipamorelin or capromorelin+2 μM SKF81297) on synaptic density in hippocampal neurons in the presence or absence of oligomeric Aβ42 (1 μM, 24 hours). **P <0.01, ***P<0.001 oligomeric Aβ42-treated neurons without agonists treatment vs with agonists treatment, two-way ANOVA followed by Bonferroni post hoc analysis. Data were collected from three independent experiments. n=24 to 30 neurites. (FIG. 9b) Representative 3D reconstructed images of synapse staining. vGLUT1 (blue) and PSD95 (red) were used to visualize pre- and postsynaptic terminals, respectively. The dendrites were stained with MAP2 (green). The overlaid staining of vGLUT1/PSD95 identifies synapses.

FIGS. 10a-b show Drd1 agonists dose test in vitro. (FIG. 9a) Effect of different treatments on synaptic density in hippocampal neurons in the presence or absence of Drd1 agonists (1 μM, 5 μM, 10 μM). ***P<0.001 vehicle vs Drd1 agonists treatment groups, two-way ANOVA followed by Bonferroni post hoc analysis. Data were collected from three independent experiments. n=27 to 30 neurites. (FIG. 9b) Representative 3D reconstructed images of synapse staining. vGLUT1 (blue) and PSD95 (red) were used to visualize pre- and postsynaptic terminals, respectively. The dendrites were stained with MAP2 (green). The overlaid staining of vGLUT1/PSD95 identifies synapses.

FIGS. 11a-b show combined ghrelin/Drd1 agonist treatments rescue hippocampal synapse in vitro. (FIG. 10a) Effect of different treatments (1 μM ghrelin+10 μM SKF38393 or 5 μM dihydrexidine) on synaptic density in hippocampal neurons in the presence or absence of oligomeric Aβ42 (1 μM, 24 hours). **P <0.01, ***P<0.001 oligomeric Aβ42-treated neurons without agonists treatment vs with agonists treatment, two-way ANOVA followed by Bonferroni post hoc analysis. Data were collected from three independent experiments. n=27 to 30 neurites. (FIG. 10b) Representative 3D reconstructed images of synapse staining. vGLUT1 (blue) and PSD95 (red) were used to visualize pre- and postsynaptic terminals, respectively. The dendrites were stained with MAP2 (green). The overlaid staining of vGLUT1/PSD95 identifies synapses.

FIGS. 12a-d3 show unchanged hippocampal Aβ deposition in MK0677-treated 5×FAD mice. (FIG. 12a) Daily MK0677 Intraperitoneal Injection on mice. (FIG. 12b) 3.0 mg/kg MK0677 treatment had significant impact on 5×FAD lifespan. Two-way ANOVA followed by Bonferroni post hoc analysis. ** P<0.01 between non-TG saline and 5×FAD 3.0 mg/kg MK0677groups, NS: no significant difference between non-TG saline and 5×FAD 1.5 mg/kg MK0677 groups, n=4-8 mice. (FIG. 12c) Mouse body weight remained the same during 1.5 mg/kg MK0677 treatment. NS=no significant difference among all the groups, n=4-8 mice. (FIGS. 12d1-d2) 1.5 mg/kg MK0677 treatment had no influence on amyloid plaque-occupied volume on 5×FAD mice. Unpaired Student's t-test. t=0.3613, NS: not significant, n=4 mice per group. (FIG. 12d2) 1.5 mg/kg MK0677 treatment did not change the single amyloid plaque volume in mice hippocampi. Unpaired Student's t-test. t=0.6155, NS: not significant, n=4 mice each group. (FIG. 12d3) Representative images for amyloid plaque staining. Scale bar=100 μm.

FIGS. 13a-b show that MK0677 treatment significantly increased neurogenesis in 5×FAD mice. (FIG. 13a) 5×FAD mice presented increased DCX⁺ cells after MK0677 treatment. Two-way ANOVA followed by Bonferroni post hoc analysis. t=3.541, * P<0.05 non-TG saline vs 5×FAD saline; t=3.172; * P<0.05 5×FAD saline vs 5×FAD MK0677; t=3.223, * P<0.05 non-TG saline vs Ghsr null saline; t=0.5837, NS: no significant difference between non-TG saline and non-TG MK0677 treatment groups; t=0.04955, NS: no difference between Ghsr null saline and MK0677 treatment groups. n=4 mice per group. (FIG. 13b) Representative images for neurogenesis, DCX⁺ cells represent newly generated neurons (red), nuclei were labeled with DAPI (blue). Scale bar=40 μm.

FIGS. 14a-b show that synaptic density remained unaltered in 5×FAD regardless of MK0677 treatment. (FIG. 14a) Saline and MK0677-treated 5×FAD mice showed similar synaptic density. Two-way ANOVA followed by Bonferroni post hoc analysis. t=7.725, *** P<0.001 between non-TG saline and 5×FAD saline groups; t=0.6671, NS: no significant difference for 5×FAD saline and MK0677 treatment groups; t=2.704, NS: no significant difference for non-TG saline and MK0677 treatment groups. n=4 mice per group. (FIG. 14b) Representative images of synapse staining. vGlutl (blue) and PSD95 (red) were used as pre- and post-synaptic markers, respectively. The overlaid staining of vGlutl and PSD95 indicates synapses. Scale bar=20 μm.

FIGS. 15a1-c3 show that MK0677 did not cause microglia-related changes in 5×FAD mice. (FIG. 15a1) Microglia density remained the same in 5×FAD mice after MK0677 treatment. Two-way ANOVA followed by Bonferroni post hoc analysis. t=8.244, *** P<0.001 between non-TG saline and 5×FAD saline groups; t=0.8793, NS: no significant difference for 5×FAD saline and MK0677 treatment groups; t=0.1297, NS: no significant difference for non-TG saline and MK0677 treatment groups. n=4 mice per group. (FIG. 15a2) 3D representative images for hippocampal microglia density. Ibal (red) antibody were used to mark microglia. Scale bar=50 p.m. (FIG. 15b1) Plaque-associated microglia density remained unchanged after MK0677 treatment in 5×FAD mice. Unpaired Student's t-test. t=2.814, NS: not significant, n=4 mice per group. (FIG. 15b2) 3D representative images generated using NIS element software. Scale bar=30 μm. (FIGS. 15c1-2) Microglia total dendrite length and branch point number did not change in 5×FAD mice with MK0677 treatment. Unpaired Student's t-test. t=0.5612 for microglia total dendrite length comparison between 5×FAD saline and MK0677 treatment groups, t=0.6058 for microglia branch points comparison between 5×FAD saline and MK0677 treatment groups. NS: not significant. n=4 mice per group. (FIG. 15c3) 3D representative images generated using Imaris software. Scale bar=40 μm.

FIGS. 16a-c show that MK0677 treatment did not improve 5×FAD mice spatial learning and memory ability. (FIG. 16a) Morris water maze learning curves had no difference between saline (0.9% NaCl) and MK0677-treated 5×FAD mice. * P<0.05, ** P<0.01 for non-TG saline and 5×FAD with and without MK0677 treatment. NS: no significant difference between 5×FAD with and without MK0677 treatment. (FIG. 16b) 5xFAD saline and MK0677 mice showed similar spatial reference memory. t=2.597, * P<0.05 between non-TG saline and 5×FAD saline treatment; t=0.3628, NS: no significant difference between 5×FAD w/, w/o MK0677 treatment; t=0.8931, NS: no significant difference between non-TG w/, w/o MK0677 treatment (FIG. 16c) Swimming speed remained the same among four experimental groups. Two-way ANOVA followed by Bonferroni post hoc analysis. t=0.3967 between non-TG saline and MK0677 treatment, t=0.6343 between non-TG and 5×FAD saline, t=0.4530 between 5×FAD saline and MK0677 treatment, NS: no significant difference among all the groups, n=5-7 mice per group.

Table 1 is a list of agonists of GHSR1α.

Table 2 is a list of agonists of DRD1.

Table S1 is a table of human brain tissue information.

FIGS. S1a-f. Expression of GHSR1α increased in hippocampi from subjects with AD and 9 month-old 5×FAD mice. (FIG. S1a) Analysis of GHSR1α expression in the hippocampal region from subjects with AD and non-AD healthy controls. Unpaired Student's t-test. ** P<0.01. n=4 per group. The right panel is representative images. Nonimmune IgG to replace specific GHSR1α antibody was used to reflect the specificity of the staining. Scale bar=200 μm. (FIG. Slb) Analysis of cell membrane-incorporated GHSR1α in the hippocampal tissues from subjects with AD and non-AD healthy controls. Unpaired Student's t-test. * P<0.05. non-AD, n=9; AD, n=8. The right panel is representative membrane blotting images. β-III-tubulin was used as the loading control. (FIG. S1c-d) Correlation between GHSR1α expression and soluble Aβ40 (FIG. S1c) (r=0.9293, P=0.0008) or Aβ42 amount (FIG. S1c) (r=0.9272, P=0.0009). Pearson correlation coefficient. n=8. (FIG. S1e) Analysis of Ghsr1α expression in the hippocampal region from 4 and 9 month-old non-TG and 5×FAD mice. Unpaired Student's t-test. NS, not significant. * P<0.05. 4 month-old mice, non-TG, n=6; 5×FAD, n=5. 9 month-old mice, non-TG, n=4; 5×FAD, n=4. The right panel are representative images. 9 month-old Ghsr null mice were used as a critical negative control. Scale bar=10 pm. (FIG. Slf) Analysis of cell membrane-incorporated Ghsr1α in the hippocampal tissues from 4 and 9 months old non-TG and 5×FAD mice. Unpaired Student's t-test. NS, not significant. * P<0.05. 4 month-old mice, non-TG, n=6; 5×FAD, n=6. 9 month-old mice, non-TG, n=7; 5×FAD, n=7. The right panel is representative membrane blotting. β-III-tubulin was used as the loading control.

FIGS. S2a-b. Expressions of Ghsr1α and Drd1 were validated in transfected HEK 293T cells. FLAG-tagged mouse Ghsr1α and HA-tagged mouse Drd1 were transiently expressed in otherwise non-expressing HEK 293T cells. The antibody specificity and expression of Ghsr1α (FIG. S2a) and Drd1 (FIG. S2b) were determined by immunofluorescence staining. The nuclei were visualized by DAPI staining. Scale bar=10 μm.

FIG. S3. Schematic diagram shows the sequence of full length Ghsr1α and its truncating mutants. Schematic map of full-length Ghsr1α, Ghsr1α aa1-67, Ghsr1α aa1-78, Ghsr1α aa1-100, Ghsr1α aa1-116, Ghsr1α Δaa42-116, Ghsr1α Δaa101-181 and Ghsr1α Δaa182-364. IL, TM and EL stand for intracellular loop, transmembrane domain and extracellular loop, respectively. A stands for deletion of fragment.

FIG. S4. FLAG-tagged Ghsr1α and its truncating mutants were similarly expressed in HEK 293T cells. Anti-FLAG-tag was used to recognize FLAG-tagged Ghsr1α and its mutants in transiently transfected HEK 293T cells, followed by incubation with Alexa Fluor 488 conjugated secondary antibody. The images were collected on a Nikon confocal microscope. The expressions of FLAG-tagged Ghsr1α and its mutants were quantified by measuring the intensity of fluorescence signals. One-way ANOVA followed by Bonferroni post hoc analysis. NS, not significant. n=8-11 cells.

FIGS. S5a-b. The interaction between Ghsr1α mutants and Aβ42 was assessed by using co-immunoprecipitation (Co-IP). (FIG. S5a) Densitometric analysis of protein interaction between oligomeric Aβ42 and Ghsr1α mutants including aa1-67, aa1-78, aa1-100, 1-116, Δaa182-364 and Δaa101-181. n=3. (FIG. S5b) Representative immunoblots showing the interactions between oligomeric Aβ42 and Ghsr1α mutants.

FIG. S6. Schematic diagram represents FlAsH-FRET assay for Ghsr1α activity and the impact of oligomeric Aβ42 on Ghsr1α activation. Ghsr1α activation was monitored by recording changes in FRET between ECFP (the donor) and FlAsH (the acceptor) introduced respectively into the C-terminal tail and the third intracellular loop of the Ghsr1α . Decreased FRET ratio in the presence of agonist reflected the activation of Ghsr1α by its agonist. Co-incubation of oligomeric Aβ42 blunted agonist-induced reduction of FRET ratio, indicating that oligomeric Aβ42 inhibits agonist-induced Ghsr1α activation.

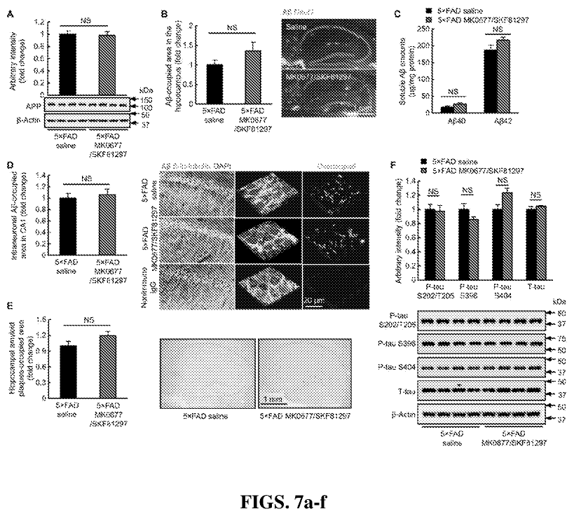

FIGS. 57a-b. GHSR1α/DRD1 complex density was negatively correlated with hippocampal soluble Aβ40 and 42 amounts in subjects with AD. GHSR1α/DRD1 complex density exhibited a strong correlation with the amounts of soluble Aβ40 (FIG. S7a) (r=−0.9003, P=0.0372) or Aβ42 (FIG. 7Sb) (r=−0.9294, P=0.0223) in hippocampi from patients with AD. Pearson correlation coefficient. n=5.

FIGS. S8a-d. Expression of hippocampal DRD1 remained unaltered in hippocampi from subjects with AD and 5×FAD mice. (FIGS. S8a-b) Analysis of DRD1 expression in the hippocampal region from subjects with AD and non-AD healthy controls. Unpaired Student's t-test. NS, not significant. n=4 cases per group. (FIG. S8b) Representative images. Nonimmune IgG to replace specific DRD1 antibody was used to determine the specificity of the staining. Scale bar=100 μm. (FIGS. S8c-d) Analysis of Drd1 expression in the hippocampal CA1 region from 4 and 9 month-old four genotypes of mice. One-way ANOVA followed by Bonferroni post hoc analysis. NS, not significant. 4 month-old mice, non-TG, n=5; 5×FAD, n=5; Ghsr null, n=3; Ghsr null/5×FAD, n=3. 9 month-old mice, non-TG, n=4; 5×FAD, n=4; Ghsr null, n=4; Ghsr null/5×FAD, n=4. (FIG. S8d) Representative images. Scale bar=10 μm.

FIGS. S9a-e. Oligomeric Aβ42 did not affect agonist-induced activation of Drd1 or form complex with Drd1 . (FIG. S9a) Oligomeric Aβ42 (2 μM) exhibited little interference with Drd1 FlAsH-FRET response in the presence or absence of Drd1 agonist SKF81297 (100 μM). Two-way ANOVA followed by Bonferroni post hoc analysis. *** P<0.001. n=18-20 per group. Moreover, the inventor's in vitro and in vivo PLA assay did not support the interaction between Aβ42 and DRD1 in hippocampal tissues from subjects with AD (FIG. S9b) or Drd1-expressing HEK 293T cells (FIGS. S9c-e). (FIG. S9b) Aβ/DRD1 PLA detection in hippocampi from AD and non-AD cases. n=4 per group. Scale bar=100 μm. (FIG. S9c) Expression of transfected Drd1-HA in HEK 293T cells. Scale bar=5 μm. (FIG. S9d) No PLA-positive dot was detected in oligomeric Aβ42-treated Drd1-HA expressing HEK 293T cells. Scale bar=5 μm. (FIG. S9e) Interaction between Drd1 and oligomeric Aβ42 was not detected in oligomeric Aβ42-treated Drd1-HA expressing HEK 293T cells by using Co-IP. Mouse HA antibody was used for immunoprecipitation of Drd1-HA. Drd1-HA immunoblots was detected with rabbit anti-HA antibody.

FIG. S10. Input/output (I/O) curves of fEPSPs were similar in four types of transgenic mice. Input/output (I/O) curves of fEPSPs were obtained by plotting the slope of fEPSPs recorded in the CA1 area of the hippocampus as a function of the stimulation intensity (from 25 to 150 μA). A one-way ANOVA followed by Bonferroni post hoc analysis revealed no significant differences between the four groups at 9 months old. NS, not significant. non-TG, n=8; 5×FAD, n=4; Ghsr null, n=6; Ghsr null/5×FAD, n=6.

FIGS. S11a-b. Aβ deposition in the hippocampus remained unchanged in Ghsr null/5×FAD mice as compared with their 5×FAD littermates. Aβ deposition in the hippocampal region was measured and analyzed from 4 (FIG. S11a) and 9 (FIG. S11b) month-old mice. Unpaired Student's t-test. NS, not significant. 4 month-old mice, 5×FAD, n=7; Ghsr null/5×FAD, n=5. 9 month-old mice, 5×FAD, n=5; Ghsr null/5×FAD, n=4. The lower panels are representative images for Aβ staining (red color). Neurons were identified by NeuN staining (green color). Scale bar=1 mm.

FIGS. S12a-b. Serum ghrelin amounts were similar in four types of transgenic mice. The amounts of serum total ghrelin in four groups of mice at 4 (FIG. S12a) and 9 (FIG. S12b) months old. One-way ANOVA followed by Bonferroni post hoc analysis. NS, not significant. 4 month-old mice, non-Tg, n=6; 5×FAD, n=6; Ghsr null, n=8; Ghsr null/5×FAD mice, n=8. 9 month-old mice, non-TG, n=8; 5×FAD, n=8; Ghsr null, n=6; Ghsr null/5×FAD mice, n=7.

FIG. S13. Loss of Ghsr1α suppressed postsynaptic CaMKII activation in the hippocampus. Analysis of CaMKII phosphorylation (Thr286) in postsynaptic density from the hippocampus of four groups of mice at 9 months old. One-way ANOVA followed by Bonferroni post hoc analysis. *** P<0.001. non-TG, n=3; 5×FAD, n=4; Ghsr null, n=3; Ghsr null/5×FAD, n=4. The lower panels are representative immunoreactive bands of phosphorylated CaMKII (P-CaMKII Thr286) and total CaMKII (T-CaMKII).

FIGS. 514a-b. The optimal doses of MK0677 and SKF81297 were determined by their augmenting effect on synaptogenesis in cultured hippocampal neurons. To determine the appropriate doses of MK0677 and SKF81297 for in vitro studies on primary hippocampal neuron culture, the inventor exposed the neurons to different doses of the indicated drugs and examined the change of synapse density. Application of MK0677 at 1.5 μM (FIG. 514a) and SKF81297 at 2 μM (FIG. 514b) greatly promoted synaptogenesis. Unpaired Student's t-test. *** P<0.001. n=33-45 neurons from 3 independent experiments. The lower panels are 3D-reconstructed representative images of synaptic staining in the presence of different doses of drugs. The pre- and post-synaptic content was determined by the staining of vGLUT1 (blue color, presynaptic marker) and PSD95 (red color, postsynaptic marker), respectively. The dendrites were identified through staining for MAP2 (green color). Scale bar=10 μm.

FIG. S15. Time course of LTP and fEPSP amplitudes was not changed by different treatments on hippocampal slices from non-TG mice. Hippocampal slices from 4 months-old non-TG mice were used for experiments. One-way ANOVA followed by Bonferroni post hoc analysis. NS, not significant. non-TG saline, n=9; non-Tg MK0677/SKF81297, n=7; non-Tg MK0677, n=7; non-TG SKF81297, n=2.

FIGS. 516a-g. The doses of MK0677/SKF81297 treatment were optimized based on the influence on body weight, serum ghrelin and behavioral performance. Non-TG and 5×FAD mice at 3 months old received daily intraperitoneal (i.p.) injections of saline or MK0677/SKF81297 combination therapy for 30 days, then the mice were subjected to behavioral experiments at 4-5 months of age. The doses of the treatment were MK0677 1 mg/kg and SKF81297 1.5 mg/kg (FIGS. S16a-b), or MK0677 3 mg/kg and SKF81297 4.5 mg/kg (FIGS. S16c-g). (FIG. S16a) The mice body weights were measured every 5 days. Two-way ANOVA followed by Bonferroni post hoc analysis. NS, not significant. non-TG saline, n=8; 5×FAD saline, n=8; non-TG MK0677/SKF81297, n=7; 5×FAD MK0677/SKF81297, n=5. (FIG. S16b) Total ghrelin amounts in mice serum were measured after treatment. Two-way ANOVA followed by Bonferroni post hoc analysis. NS, not significant. non-TG saline, n=6; 5×FAD saline, n=6; non-TG MK0677/SKF81297, n=7; 5×FAD MK0677/SKF81297, n=5. (FIG. S16c) Body weight was measured every 5 days. Two-way ANOVA followed by Bonferroni post hoc analysis. NS, not significant. (FIG. S16d) Ghrelin amount in mice serum was measured after treatments. Two-way ANOVA followed by Bonferroni post hoc analysis. NS, not significant. (FIGS. S16e-g) Spatial navigation analysis in four groups of mice treated with vehicle (saline) or MK0677/SKF81297 performing the Morris water maze test. (FIG. S16e) Spatial learning. Two-way ANOVA followed by Bonferroni post hoc analysis. There is no significant difference (NS) between 5×FAD saline and 5×FAD MK0677/SKF81297. (FIG. S16e) Spatial reference memory. Two-way ANOVA followed by Bonferroni post hoc analysis. ** P<0.01, *** P<0.001. (FIG. S16g) Swimming speed. Two-way ANOVA followed by Bonferroni post hoc analysis. NS, not significant. non-TG saline, n=4; 5×FAD saline, n=8; non-TG MK0677/SKF81297, n=7; 5×FAD MK0677/SKF81297, n=4.

FIGS. S17a-c. MK0677/SKF81297 (MK0677 1 mg/kg; SKF81297 1.5 mg/kg) treatment on mice had little effect on hepatic, renal and hippocampal cell density. The cell density in liver (FIG. S17a), kidney (FIG. S17b) and hippocampus (FIG. S17c) in different groups of mice with saline or MK0677/SKF81297 treatment. Two-way ANOVA followed by Bonferroni post hoc analysis. NS, not significant. n=5 mice per group. The right panels are the representative images of HE staining, scale bar=100 μm (FIG. S17b),=1 mm (FIG. S17c).

FIG. S18. MK0677/SKF81297 (MK0677 1 mg/kg; SKF81297 1.5 mg/kg) treatment improved neurogenesis in the dentate gyrus of 5×FAD mice. The number of doublecortin (DCX)-positive neurons were counted in the dentate gyms from four groups of mice. Two-way ANOVA followed by Bonferroni post hoc analysis. ** P<0.01. n=3 mice per group. The lower panel are 3D-reconstructed representative images. Neurons were identified by the staining of NISSL blue (blue color) and MAP2 (green color). Adult neurogenesis was determined by DCX positive staining (red color). Scale bar=40 μm.

FIGS. S19a-c. MK0677 treatment did not influence cortical amyloid plaque deposition. (FIGS. S19a) MK0677 treatment did not change amyloid plaque-occupied volume on 5×FAD mice. Unpaired Student's t-test. t=1.059, NS: not significant, n=4 mice per group. (FIG. S19b) Cortical single amyloid plaque volume remained the same in 5×FAD mice with MK0677 treatment. Unpaired Student's t-test. t=1.982, NS: not significant, n=4 mice per group. (FIG. S19c) Representative images for amyloid plaque staining. Scale bar=200 μm.

FIGS. S20a-b2. MK0677 did not cause microglia related changes in 5×FAD mice cortex. (FIG. S20a1) Microglia density remained the same in 5×FAD mice cortex after MK0677 treatment. Unpaired Student's t-test. t=1.704, NS: not significant, n=4 mice per group. (FIG. S20a2) 3D representative images for hippocampal microglia density. Thal (red) were used to stain microglia. Scale bar=10 μm. (FIG. S20b1) Cortical Plaque-associated microglia density remained unchanged after MK0677 treatment in 5×FAD mice. Unpaired Student's t-test. t=0.7636, NS: not significant, n=4 mice per group. (FIG. S20b2) 3D representative images for cortical plaque-associated microglia density. Scale bar=20 μm.

### DETAILED DESCRIPTION

### A. ALZHEIMER'S DISEASE

Alzheimer's Disease (AD) is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40-residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes the protease responsible for cleaving APP into Aβ. There are two forms—PS1 and PS2. Mutations in PS1, causing production of Aβ42, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

### B. PHARMACEUTICAL FORMULATIONS AND ROUTES OF ADMINISTRATION

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render drugs stable and allow for uptake by target cells. Buffers may be employed when drugs are introduced into a patient. Aqueous compositions of the present disclosure comprise an effective amount of the drug to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the drugs of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration, the compounds of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### C. COMBINATION THERAPY

Compounds of the disclosure may be used in combination with other drugs that are known to be useful in the treatment or amelioration of the diseases or similar diseases. In the combination administration, such other drugs may be administered, by a route administration and in an amount commonly used, and contemporaneously or sequentially with a compound of Formula. When a compound of Formula (I) is used contemporaneously with one or more other drugs, a pharmaceutical composition containing one or more other known drugs and the compound of Formula (I) is preferred.

To treat a subject using the methods and compositions of the present disclosure, one would generally contact a “target” cell or subject with an agent according to the present disclosure and at least one other agent. These compositions would be provided in a combined amount effective to treat the disease. This process may involve contacting the cells with the agent according to the present disclosure and the other treatment at the same time. This may be achieved by contacting the cell with a single composition or pharmacological formulation that includes both agents, or by contacting the cell with two distinct compositions or formulations, at the same time, wherein one composition includes the agent according to the present disclosure and the other includes the other agent.

Alternatively, the agent according to the present disclosure may precede or follow the other agent treatment by intervals ranging from minutes to weeks. In embodiments where the other agent and the agent according to the present disclosure are applied separately to the cell, one would generally ensure that a significant period of time did not expire between the time of each delivery, such that the agent according to the present disclosure and the other therapy would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the agent according to the present disclosure or the other therapy will be desired. Various combinations may be employed, where an agent according to the present disclosure therapy is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve an effective treatment, such as slowing the progression of AD, reducing the amount of formation of Aβ plaques, or improving cognitive abilities, both agents are delivered in a combined amount effective to achieve such clinical endpoints.

As mentioned above, Aricept (donepezil) and Exelon (rivastigmine) are reversible acetylcholinesterase inhibitors that are indicated for the treatment of mild to moderate dementia of the Alzheimer's type. Cholesterol reducing agents have also been suggested as useful in treating AD. Other medications may help control behavioral symptoms such as sleeplessness, agitation, wandering, anxiety, and depression, thus making the patient more comfortable. These are all non-limiting examples of suitable combination therapies with the compounds of this disclosure.

### D. EXAMPLES

The following examples are included to demonstrate preferred embodiments. It should be appreciated by those of skill in the art that the techniques disclosed in the examples which follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Results

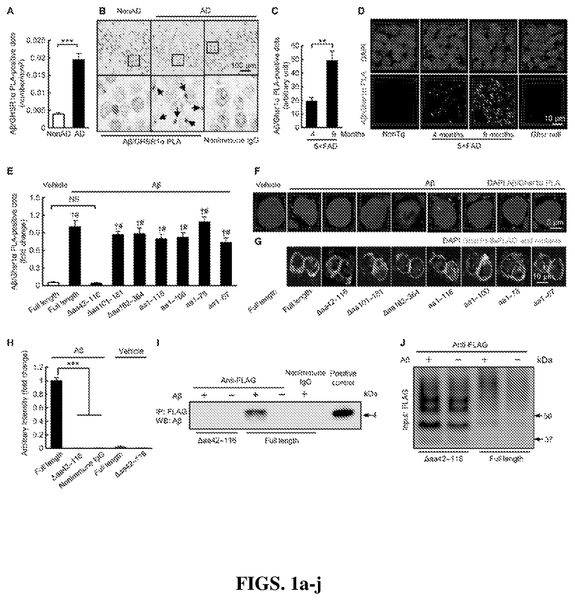

Aβ3 physically interacts with GHSR1α. To determine whether GHSR1α expression is changed in the hippocampus in AD, the inventor performed immunohistochemical and membrane blotting assays in post-mortem hippocampal tissues from 4 subjects with AD and 4 non-demented healthy donors (non-AD). He observed increased GHSR1α expression in hippocampal tissues from patients with AD (FIGS. S1a-b and Table S1), which positively correlated with the amounts of soluble Aβ40 and Aβ42 in hippocampi (FIGS. S1c-d). Increased hippocampal Ghsr1α expression was prominent in 5×FAD mice especially at 9 months old (FIGS. S1e-f), when the mice demonstrate heavy brain amyloidopathy with severe hippocampal lesions (13). These results suggest a potential relationship between GHSR1α expression and Aβ toxicity. Because Aβ binds to multiple proteins (14-16), the inventor next explored whether GHSR1α is an Aβ binding target. In order to examine the interaction between GHSR1α and AP, he labeled GHSR1α and Aβ with their specific antibodies and ran Duolink proximity ligation assay (PLA), which is a sensitive method to visualize and quantify direct protein interactions in situ (17), on hippocampal tissues from 4 subjects with AD and 4 healthy donors. The inventor observed Aβ3/GHSR1α complexes in hippocampi from patients with AD (FIGS. 1a-b). Moreover, 5×FAD mice at 4 and 9 months old exhibited increased hippocampal Aβ/Ghsr1α complexes in an age-dependent manner (FIGS. 1c-d). To validate the specificity of this interaction, the expressed full-length mouse Ghsr1α or its truncating mutants (FIGS. S2-3) in otherwise non-Ghsr1α-expressing human embryonic kidney (HEK 293T) cells, and the cells exhibited similar expression of FLAG-tagged Ghsr1α and its mutants (FIG. 1g, FIG. S4). HEK 293T cells expressing full-length mouse Ghsr1α or its mutants were exposed to 5 μM oligomeric Aβ42 for 24 hours followed by Duolink PLA to detect Aβ/Ghsr1α complexes. In contrast to full-length Ghsr1α and other tested Ghsr1α mutants (FIGS. 1e-f), Ghsr1α mutant devoid of aa42-116 (Ghsr1α Δaa42-116) showed no interaction with oligomeric Aβ42 (FIGS. 1e-f). The Duolink PLA results were further validated by using coimmunoprecipitation (Co-IP) (FIGS. 1h-j, FIGS. S5a-b). These results confirm the interaction between the two proteins as seen in AD and further suggest that aa42-116 residues on Ghsr1α are critical for Aβ binding. Taken together, these findings indicate that Aβ physically interacts with GHSR1α in AD.

TABLE S1
Human brain tissues information

| | | | | | | CERAD |
|---|---|---|---|---|---|---|
| Clinical Dx | Case number | Gender | Age | PMI(Hr) | Braak | score |
| non-AD | 36359 | M | 84 | 24 | IV | Normal |
| non-AD | 42133 | F | 100 | 12 | IV | Normal |
| non-AD | 42990 | F | 84 | 14 | I | Normal |
| non-AD | 45329 | M | 78 | 21 | I | Normal |
| non-AD | 39146 | F | 67 | 12 | II | Normal |
| non-AD | 46202 | M | 77 | 20 | II | Normal |
| non-AD | K45 | M | 66 | 18 | N/A | Normal |
| non-AD | K46 | M | 79 | 17 | I | Normal |
| non-AD | K101 | M | 76 | 21.25 | N/A | Normal |
| non-AD | K108 | F | 75 | 20 | 0 | Normal |
| Mean ± SE | | 4F/6M | 78.4 ± 2.8 | 18.61 ± 1.6 | | |
| AD | 46090 | F | 75 | 27 | VI | Definite |
| AD | 46121 | F | 74 | 16 | VI | Definite |
| AD | 46991 | M | 62 | 23 | V | Definite |
| AD | 47586 | F | 78 | 18 | V | Definite |
| AD | K01 | M | 84 | 15.5 | V | Definite |
| AD | K26 | F | 79 | 10 | V/VI | Definite |
| AD | K44 | M | 87 | 17.75 | V/VI | Probable |
| AD | K55 | F | 87 | 28.5 | V | Definite |
| Mean ± SE | | 5F/3M | 78.25 ± 2.93 | 19.49 ± 2.21 | | |

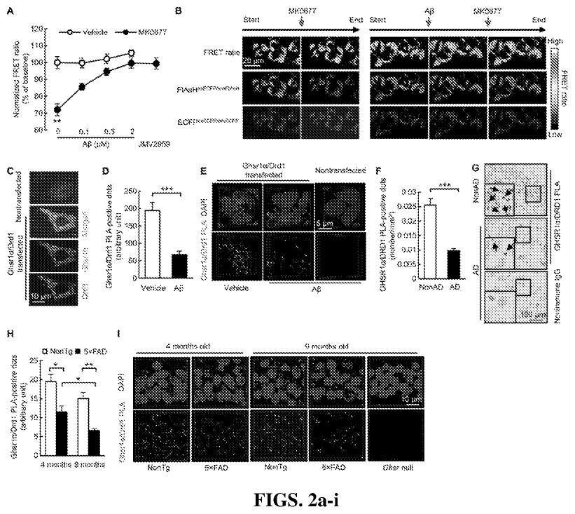

The interaction with Aβ induces GHSR1α dysfunction. To determine whether s interaction affects the function of Ghsr1α, the inventor examined Ghsr1α activity in Aβ-enriched environments by using fluorescein arsenical hairpin binder (FlAsH)-based fluorescence resonance energy transfer (FRET) assay, which is advantageous for GPCR activity assay because of its minimally-perturbing effect on GPCR function (18). FlAsH-based FRET was created by structural dynamics of Ghsr1α with FlAsH in the intercellular loop 3 and enhanced cyan fluorescent protein (ECFP) in the C-terminus (Ghsr1α^{FlAsH/ECFP}). Ghsr1α^{FlAsH/ECFP} was expressed in HEK 293T cells and changes in FRET ratio (F_{FlAsH}/F_{ECFP}) was monitored for the measurement of agonist-induced Ghsr1α activation. No change in FRET ratio was detected in vehicle-treated Ghsr1α^{FlAsH/ECFP}-expressing HEK 293T cells, whereas the administration of MK0677 (50 μM) induced a decrease in FRET ratio (FIGS. 2a-b). Ghsr1α antagonist, JMV2959 (50 μM) diminished MK0677-mediated FRET ratio decrease (FIG. 2a). These results indicate that agonist-induced Ghsr1α activation results in less energy transfer between ECFP and FlAsH (FIGS. 2a&b). Oligomeric Aβ42 at indicated concentrations was applied on Ghsr1α^{FlAsH/ECFP}-expressing HEK 293T cells for 5 minutes (pre-incubation) followed by co-incubation with vehicle or Ghsr1α agonist MK0677. Although oligomeric Aβ42 had no impact on FRET ratio in vehicle-treated cells, agonist-induced Ghsr1α activation was suppressed by oligomeric Aβ42 (FIGS. 2a-b), suggesting a Ghsr1α antagonist-like effect of oligomeric Aβ42. Therefore, oligomeric Aβ42 reduced the response of Ghsr1α to activation (FIG. S6) and that this effect was likely to be due at least partially to Aβ/Ghsr1α interaction.

GHSR1α forms a complex with DRD1 to regulate DRD 1-mediated hippocampal synaptic strength and memory (8). In order to fully evaluate the influence of Aβ on GHSR1α, the inventor next examined whether oligomeric Aβ42 affects heterodimerization of GHSR1α and DRD1 by using HEK 293T cells co-expressing Ghsr1α and Drd1 (FIG. 2c, FIG. S2). After an incubation with oligomeric Aβ42 (5 μM) for 24 hours, the interaction between Ghsr1α and Drd1 determined by Duolink PLA was suppressed (FIGS. 2d-e), indicating the inhibitory effect of oligomeric Aβ42 on Ghsr1α/Drd1 heterodimerization. To test whether GHSR1α/DRD1 interaction was modulated in a clinical setting, the inventor examined hippocampal tissues from 5 patients with AD and compared with tissues from 5 healthy controls. Analysis of Duolink PLA data showed a reduction of GHSR1α/DRD1 complexes in hippocampi from subjects with AD compared with those from control subjects (FIGS. 2f-g), with a negative correlation between GHSR1α/DRD1 complex density and the amounts of hippocampal soluble Aβ40 and 42 (FIGS. S7a-b). Decreased Ghsr1α/Drd1 heterodimerization also occurred in 5×FAD mice, and this effect was exacerbated with age (FIGS. 2h-i). The preserved expression of DRD1 in hippocampi from subjects with AD (FIGS. S8a-b) and 5×FAD mice (FIGS. S8c-d) suggests that decreased GHSR1α/DRD1 interaction in AD-relevant pathological settings is not due to DRD1 loss. FlAsH-based FRET assay was performed to examine the effect of oligomeric Aβ42 (2 μM minutes preincubation) on agonist (SKF81297 100 μM)-induced Drd1 activity in HEK 293T cells expressing Drd1 with FlAsH in the intercellular loop 3 and ECFP in the C-terminus (Drd1^{FlAsH/ECFP}). Unaltered agonist—induced Drd1 activation examined by FlAsH-based FRET indicates no impact of oligomeric Aβ42 on Drd1 activation (FIG. S9a). Moreover, Aβ/DRD1 complexes were not observed in hippocampi from patients of AD (FIG. S9b) by using Duolink PLA or oligomeric Aβ42 (5 M, 24 hours)-exposed Drd1-expressing HEK 293T cells by using Duolink PLA (FIGS. S9c-d) and Co-IP assays (FIG. S9e). Therefore, decreased hippocampal GHSR1α/DRD1 interaction in AD-relevant conditions most likely results from Aβ-mediated GHSR1α deregulation.

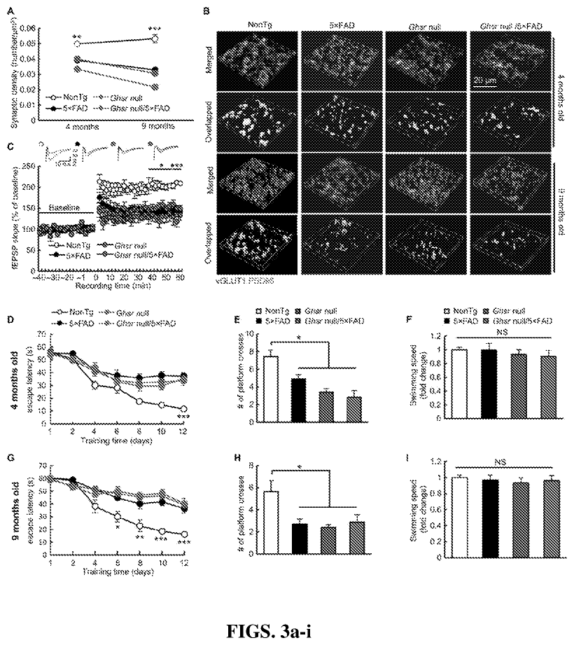

Loss of Ghsr1α induces AD-like hippocampal synaptic stress and memory deficits. Because GHSR1α function is critical for hippocampal synaptic physiology (19), the inventor hypothesized that Ghsr1α-deficient mice would display AD-like synaptic loss in the hippocampus and cognitive impairment. To test this hypothesis, the inventor measured synaptic density in hippocampal slices from Ghsr null (20), non-transgenic (non-Tg), 5×FAD and Ghsr null/5×FAD mice at 4 and 9 months old. Ghsr null mice demonstrated reduced synapse density (FIGS. 3a-b) in the hippocampal CA1 region, one of the areas afflicted in AD with Ghsr1α abundance (21, 22). The effect of the lack of Ghsr signaling on synaptic density is in agreement with a previous report (19). The inventor next examined long-term potentiation (LTP) in the hippocampal CA3-CA1 pathway (15) to determine the effect of Ghsr loss on synaptic strength. He found impairments of stimulus-evoked LTP on hippocampal slices from 9 month-old Ghsr null mice demonstrated by decreased field excitatory postsynaptic potential (fEPSP) slope after theta burst stimuli at 40-60 minutes (FIG. 3c) without altering baseline input-output relationships of the evoked responses (FIG. S10). Moreover, loss of Ghsr impaired hippocampus-dependent spatial navigation in the Morris water maze test (FIGS. 3d, 3e, 3g, 3h) without affecting mouse swimming speed (FIGS. 3f, 3i). These changes in Ghsr null mice were in line with phenotypic alterations observed in age- and gender-matched 5×FAD mice (FIGS. 3d-i). Ghsr null/5×FAD showed no major differences in the tested parameters relative to their age- and gender-matched 5×FAD littermates (FIGS. 3a-i), and no differences in hippocampal AP loading (FIGS. S11a&b) or serum ghrelin (FIGS. S12a&b). These results suggest that the impairments observed in Ghsr null and in 5×FAD mice might be mediated by overlapping mechanisms. The regulation of DRD1 by GHSR1α is pivotal for hippocampal synaptic plasticity, which is modulated through the activation of CaMKII downstream of DRD1 signaling (8). To determine whether impaired CaMKII activation due to Ghsr1α deregulation is involved in hippocampal synaptic deficits in 5×FAD mice, the inventor examined CaMKII phosphorylation at Thr286 (P-CaMKIIα Thr286), an activated form of CaMKII (23), in postsynaptic densities isolated from mouse hippocampus by immunoblotting to reflect CaMKIIα activation at hippocampal postsynapses. Decreased CaMKII phosphorylation at Thr286 was detected in hippocampal postsynaptic densities from 9 month-old Ghsr null, 5×FAD and Ghsr null/5×FAD mice as compared with their non-Tg littermates (FIG. S13). Of note, Ghsr1α deficiency did not affect the expression of hippocampal Drd1 , regardless of Aβ overexpression (FIGS. S8c-d). These results implicate the detrimental effect of Ghsr1α deregulation on Drd1-related CaMKII signaling in 5×FAD mice. Altogether, these findings support the idea that Aβ-induced Ghsr1α deregulation underpins hippocampal synaptic deficits and cognitive decline in 5×FAD mice.

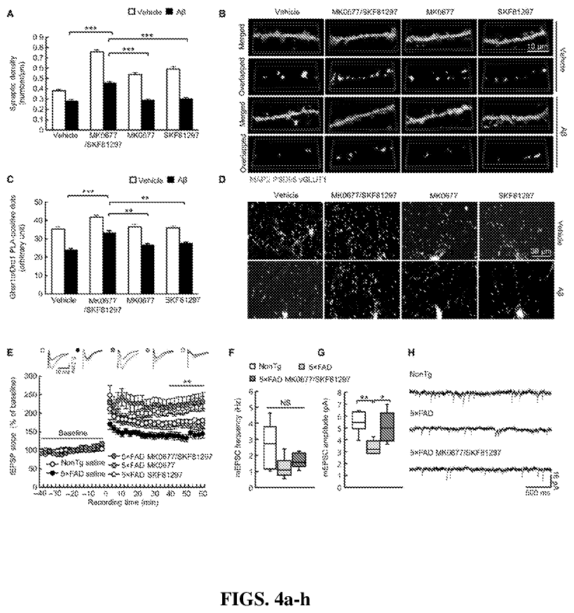

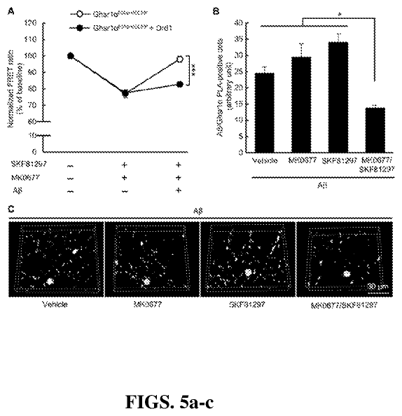

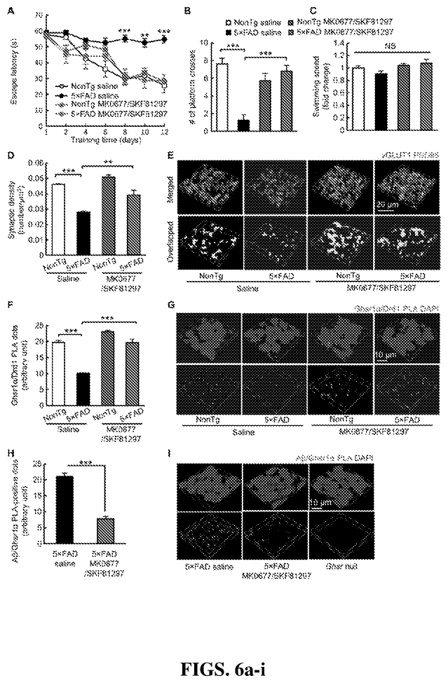

Reduced Ghsr1α/Drd1 interaction contributes to An-induced hippocampal synaptic injury. Given the deleterious influence of the GHSR1α deficiency on hippocampal synapses, the inventor asked whether GHSR1α activation could restore synaptic function in Aβ-rich environments. To directly test this, he applied the Ghsr1α agonist MK0677 to oligomeric Aβ42-exposed hippocampal neuron cultures. The inventor's interest in MK0677 derives from the translational potential of MK0677, a non-peptide ghrelin mimetic compound with higher potency than ghrelin (24). The agonist MK0677 alone at doses greater than 1.5 μM substantially promoted synaptic formation demonstrated by increased synaptic density (FIG. S14a), suggesting that Ghsr1α activity can induce synaptogenesis. However, administration of MK0677 (1.5 μM) did not mitigate oligomeric Aβ42 (1 μM, 24 hours)-induced synapse loss in hippocampal neuron cultures (FIGS. 4a-b), which is consistent with the inventor's finding that oligomeric Aβ42 blunts MK0677-mediated Ghsr1α activation. Because MK0677 alone was not protective against oligomeric Aβ42-induced synapse loss, the inventor further explored the importance of Ghsr1α/Drd1 interaction. He sought to promote Ghsr1α regulation of Drd1 by increasing Ghsr1α/Drd1 heterodimerization through simultaneously activating Ghsr1α and Drd1 in cultured mouse hippocampal neurons. The optimal dose for the selective Drd1 agonist SKF81297 (2 μM) was determined based on its capability in promoting synapse formation in cultured mouse hippocampal neurons (FIG. S14b). Same as MK0677, SKF81297 itself increased synaptic density in cultured mouse hippocampal neurons but failed to alleviate oligomeric Aβ42 (1 μM, 24 hours)-induced synapse loss in hippocampal neuron cultures (FIGS. 4a-b). In contrast to MK0677 or SKF81297 alone, the simultaneous stimulation of Ghsr1α and Drd1 using both compounds (1.5 μM MK0677 and 2 μM SKF81297) increased synaptic density in oligomeric Aβ42 (1 μM, 24 hours)-treated mouse hippocampal neuron cultures (FIGS. 4a-b). Consistent with this observation, co-application of MK0677 and SKF81297, but not the agonists alone, preserved Ghsr1α/Drd1 complex formation from oligomeric Aβ42 (1 μM, 24 hours) (FIGS. 4c-d). These results indicate that interaction of Ghsr1α/Drd1 promotes synaptic formation in hippocampal neurons, which is in agreement with previous reports (8), and they further support the hypothesis that perturbed GHSR1α regulation of DRD1 contributes to synaptic injury in AD.

To verify this hypothesis in Aβ overexpression-mediated model of synaptic injury, the inventor applied MK0677 (1.5 μM) and SKF81297 (2 μM), alone or in combination, on hippocampal slices from 4 month-old non-Tg and 5×FAD mice and examined LTP to reflect synaptic strength in the hippocampal CA3-CA1 pathway. Previous studies (13) showed that 5×FAD mice present early hippocampal synaptic lesions at 4 months of age. Vehicle-treated 5×FAD hippocampal slices exhibited impairments of stimulus-evoked LTP (FIG. 4e), indicating decreased synaptic strength. The treatment of MK0677 or SKF81297 alone had no effect on hippocampal LTP in 5×FAD hippocampal slices (FIG. 4e). In contrast, simultaneous application of MK0677 and SKF81297 markedly mitigated impairments of stimulus-evoked LTP in 5×FAD hippocampal slices (FIG. 4e). Of note, the treatment of MK0677 or SKF81297 alone or in combination had no effect on hippocampal LTP in brain slices from non-Tg mice (FIG. S15). In view of damaged excitatory synaptic transmission in AD (25), the inventor next examined the effect of MK0677/SKF81297 mixture on excitatory synaptic transmission in the hippocampus of 5×FAD mice at 4 months old by performing whole-cell recordings of miniature excitatory postsynaptic currents (mEPSCs). Although no genotypic effect on mEPSC frequency was observed (FIGS. 4f, 4h), CA1 neurons from 5×FAD mice demonstrated decrease in mEPSC amplitude, which was protected by the MK0677/SKF81297 mixture (FIGS. 4g, 4h). Because mEPSC frequency primarily represents the probability of presynaptic release and mEPSC amplitude is largely associated with the conductance of postsynaptic receptors (26), the results suggest improved postsynaptic receptor function in MK0677/SKF81297-treated 5×FAD CA1 neurons. Taken together, these findings strongly suggest a role for dysfunctional GHSR1α regulation of DRD1 in AD hippocampal synaptic failure, and also indicate that combined stimulation of GHSR1α and DRD1 can rescue Aβ-induced hippocampal synaptic deficits.

Ghsr1α/Drd1 co-activation rescues Ghsr1α function from Aβ toxicity. In order to assess whether the protective effects of Ghsr1α and Drd1 co-activation was mediated by Ghsr1α activity, the inventor measured Ghsr1α activity by FlAsH-based FRET on HEK 293T cells either expressing Ghsr1α^{FlAsH/ECFP} alone or co-expressed with Drd1 . The cells were exposed to vehicle treatment or the mixture of MK0677 (50 μM) and SKF81297 (100 μM) in the presence or absence of a 5 minutes' pretreatment of 2 μM oligomeric Aβ42. Without oligomeric Aβ42 the two types of cells exhibited similar response to the combined treatment (FIG. 5a), indicating that Ghsr1α was activated by its agonist regardless of Drd1 expression. Of note, the inhibitory effect of oligomeric Aβ42 on Ghsr1α activation was diminished in MK0677/SKF81297 mixture-treated Ghsr1α/Drd1 co-expressing cells (FIG. 5a). These results suggest that co-activation of Ghsr1α and Drd1 can prevent Aβ-induced effects on Ghsr1α . In further support of this hypothesis, the MK0677/SKF81297 mixture but not MK0677 or SKF81297 alone alleviated Aβ/Ghsr1α complex formation in oligomeric Aβ42 (1 μM, 24 hours)-treated mouse hippocampal neuron cultures (FIGS. 5b,c). Taken together, these results suggest that Ghsr1α/Drd1 co-activation preserves Ghsr1α activity by reducing the interaction between Aβ and Ghsr1α.

Ghsr1α/Drd1 co-activation rescues synaptic density and memory in 5×FAD mice. Next, the inventor attempted to replicate these findings in vivo. Because 5×FAD mice begin to exhibit compromised spatial learning and memory at 4-5 months old (27-29), he expected that Ghsr1α/Drd1 co-activation would restore hippocampal synaptic function and improve behavior in young 5×FAD mice at 4-5 months old, when hippocampal lesions are limited and AP accumulation is low (27-29). Age- and gender-matched non-Tg and presymptomatic 5×FAD mice (“presymptomatic” is referred to unaffected spatial learning and memory) at 3 months old received daily intraperitoneal (i.p.) injections of the MK0677/SKF81297 combination therapy (MK0677 1 mg/kg; SKF81297 1.5 mg/kg) for 30 days followed by behavioral experiments at 4 months of age. These treatment regimens were optimized based on preliminary experiments that took into account the influence on body weight (FIGS. S16a, S16c), serum ghrelin (FIGS. S16b, S16d) and behavioral performance (FIGS. 6a-c, FIGS. S6e-g) as well as previous reports (30, 31). Saline-treated 5×FAD mice demonstrated memory defects in the Morris water maze test, which were prevented by treatment with MK0677/SKF81297 (FIGS. 6a-c). Moreover, mice that received MK0677/SKF81297 treatment maintained bodyweight (FIG. S16a) and serum ghrelin amount (FIG. S16b) and showed unaffected cell density in the liver, kidney and brain (FIG. S17). 5×FAD mice treated with MK0677/SKF81297 showed considerably less hippocampal CA1 synapse loss (FIGS. 6d-e), preserved Ghsr1α/Drd1 heterodimerization (FIGS. 6f-g), and fewer Aβ3/Ghsr1α complexes (FIGS. 6h-i) as compared with their vehicle-treated counterparts. Additionally, because Ghsr1α activation has been shown to improve hippocampal neurogenesis in 5×FAD mice (32), the inventor examined neurogenesis in the dentate gyms by performing immunocytochemistry staining of adult brain neurogenesis marker, Doublecortin (DCX) (33). Compared with their vehicle-treated counterparts, MK0677/SKF81297-treated 5×FAD mice exhibited increased DCX-positive neurons in their dentate gyms (FIG. S18).

Ghsr1α/Drd1 co-activation does not affect hippocampal amyloidosis or Tau pathology in 5×FAD mice. To determine whether synaptic function and memory improvement in MK0677/SKF81297-treated 5×FAD mice was associated with altered Aβ production and deposition, the inventor examined hippocampal tissues from MK0677/SKF81297- and vehicle-treated 5×FAD mice. Immunoblotting assay using antibody against amyloid-beta precursor protein (APP) was performed to examine APP expression in hippocampal homogenates (FIG. 7a). Immunohistochemical staining using antibody against Aβ was performed to detect Aβ load on hippocampal slices (FIG. 7b). The amounts of soluble Aβ40 and Aβ42 in hippocampal homogenates were measured by ELISA assay (FIG. 7c). Moreover, because 5×FAD mice have intra-neuronal Aβ deposition in addition to extracellular plaques mimicking AD brain amyloidosis (13), the inventor further examined intraneuronal Aβ deposition in hippocampal CA1 neurons demonstrated by the overlapping staining of Aβ and class III β-tubulin, a specific neuronal marker (34) (FIG. 7d), as well as extracellular amyloid plaques in the hippocampus determined by Congo red-positive staining (FIG. 7e). No difference in these parameters was observed between MK0677/SKF81297- and vehicle-treated 5×FAD mice (FIGS. 7a-e). Additionally, to determine whether MK0677/SKF81297 treatment affects tau pathology, the inventor analyzed tau phosphorylation by performing immunoblotting on mice hippocampal homogenates. Tau phosphorylation at multiple phosphorylation sites including S202/T205, S396 and S404 or total tau in 5×FAD mice were not changed by MK0677/SKF81297 treatment (FIG. 7f). Therefore, the protective effects of MK0677/SKF81297 treatment do not result from modulation on brain amyloidosis or Tau pathology.

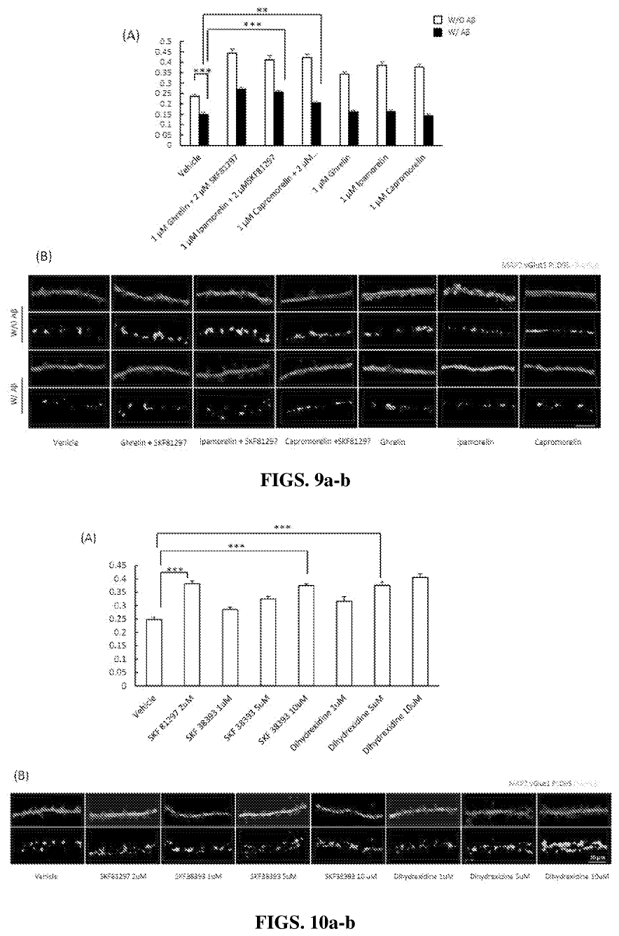

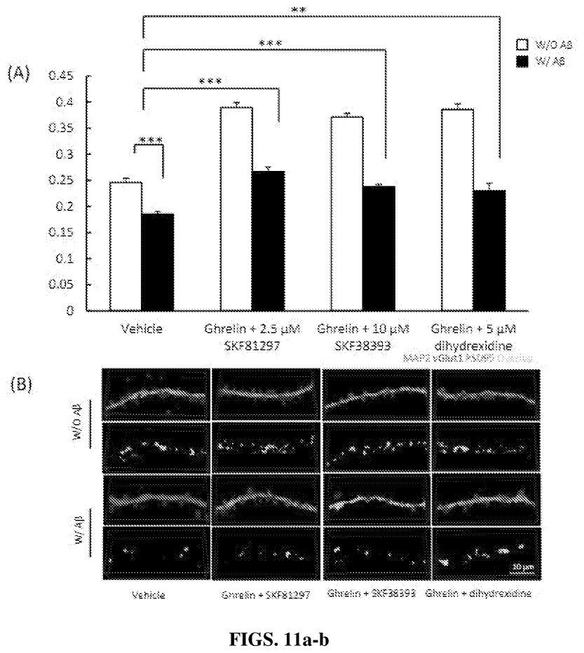

Co-activation of Ghsr1α/Drd1 rescues Aβ-induced hippocampal synaptic injury. The inventor has tested the protective effect of MK677 and SKF81297. He then sought to examine whether other GHSR1α and DRD1 agonists have similar protection against Aβ toxicity-induced synaptic injury. To this end, the inventor first tested two peptide agonists of GHSR1α (MK677 is a compound agonist) including ipamorelin and capromorelin. The concentrations of ipamorelin and capromorelin were optimized to display comparable synaptogenic effect with ghrelin, the natural ligand of GHSR1α (FIGS. 9a-b). In the presence of oligomeric Aβ42 (1 μM, 24 hours), either ipamorelin or capromorelin alone could ameliorate Aβ-induced synaptic loss on cultured hippocampal neurons (FIGS. 9a-b), which is in agreement with the inventor's previous observation with MK677⁶⁹. Of note, the mixture of SKF81297 with either ipamorelin or capromorelin demonstrated significant protection on synaptic density from Aβ toxicity (FIGS. 9a-b). Furthermore, the inventor tested two DRD1 agonists including SKF38393 and dihydrexidine⁷⁰⁻⁷³. Different from SKF81297 and SKF38393, which are benzazepine derivatives and selectively bind DRD1, dihydrexidine binds both DRD1 and DRD2 but with a much weaker binding affinity with the latter one. After optimizing the doses of SKF38393 and dihydrexidine based on their capability in promoting synaptogenesis (FIGS. 10a-b), the inventor applied the two drugs at appropriate dosages in combination with ghrelin to cultured hippocampal neurons in the presence or absence of oligomeric Aβ42 (1 μM, 24 hours). In contrast to SKF38393 or dihydrexidine alone, mixed use of ghrelin with either SKF38393 or dihydrexidine exhibited substantial effect on preserving synaptic density from Aβ toxicity (FIGS. 11a-b). These results are consistent with the inventor's previous finding that the simultaneous use of MK0677 and SKF81297 protects synapses on Aβ-insulted hippocampal neurons. Moreover, based on the use of different classes of drugs to confirm that such a protection is largely associated with the co-activation of GHSR1α/Drd1 , the inventor proposes that the combination of selective DRD1 and GHSR1α agonists at appropriate ratio is a promising AD therapy that confers protection on hippocampal synaptic strength against Aβ toxicity.

### Discussion

Recent studies highlighted the importance of GHSR1α in hippocampal synaptic physiology (8, 19, 35), but the functional status of GHSR1α in AD remains largely unknown. In this study, the inventor found elevated expression of GHSR1α in the hippocampus from patients with AD and in 5×FAD mice. The inventor's recent observation of an inverse relationship between serum acylated ghrelin amounts and cognitive function in MCI (12), as well as the findings here showing that Aβ alters the response of Ghsr1α to its agonist and that a strong correlation of GHSR1α expression with soluble Aβ amounts in subjects with AD exists, seem to suggest that increased GHSR1α expression in hippocampi from patients with AD might reflect a compensatory response to Aβ toxicity. These results are in disagreement with a previous report showing decreased GHSR1α mRNA in temporal gyri from patients with AD (36). This difference may result from different mechanisms of regulation of GHSR1α at the pre- and post-transcriptional steps and/or a brain region-specific response to Aβ toxicity. Because GHSR1α expression is relatively low in the neocortex (21), it is unclear whether the decreased GHSR1α mRNA expression in the neocortical temporal lobe contributes to AD. Furthermore, it should be noted that GHSR1α, a truncated splice variant of GHSR1α, blocks GHSR1α function (37) and the aforementioned study reported increased GHSR1α mRNA in neocortical temporal tissues from subjects with AD (36). Therefore, GHSR1α may also contribute to hippocampal GHSR1α deregulation in AD. Additional studies are needed to address these questions, and to understand the contribution of the GHSR/ghrelin system in AD pathogenesis. Here, the inventor shows that hippocampal GHSR1α deregulation can be induced through a physical interaction with AP, and he established a link between Ghsr1α deregulation, hippocampal synaptic injury, and cognitive impairments in mice. The alterations in Ghsr1α in young 5×FAD mice and the abnormal increase of acylated ghrelin in patients with MCI (12) seem to suggest that GHSR1α deregulation may develop in prodromal or early stages of the disease. Studies of postmortem tissues from preclinical patients with AD could help to address this possibility.

Substantial efforts are currently directed towards the development of new AD treatments, especially towards the development of disease-modifying therapies (38). However, current AD interventions, including acetylcholinesterase (AChE) inhibitors or N-methyl-D-aspartate receptor (NMDAR) blockers, do not target the underlying mechanisms that cause synaptic injury, and thus have limited efficacy (2, 39, 40). Moreover, ongoing attempts to remove Aβ or ameliorate tau pathology have yet to prove effective (41, 42). These results identify GHSR1α, and particularly its interaction with DRD1, as a target for AD treatment with translational potential. Studies that previously explored GHSR1α agonism for the treatment of AD produced inconsistent results (10, 11). GHSR1α agonists such as MK0677 and LY444711 provided protection in animal and cell models (43-46); however, a clinical trial of MK0677 in patients with AD failed to show clinical benefits (10). Although this trial was originally designed to enhance brain Aβ clearance by augmenting insulin-growth factor 1 (IGF-1) release (10), its negative outcome discouraged further attempts to target GHSR1α in AD. The inventor speculates here that a potential explanation for this clinical trial's failure may be that GHSR1α becomes insensitive to its agonists in AD. Previous studies found ghrelin (45) or acylated ghrelin (47) protective against acute Aβ-induced synaptic dysfunction, cognitive impairments, and neuroinflammation. Discrepancies between these studies and ours may reflect differences in the degree of Aβ exposure, as Aβ overexpression in transgenic mouse models of AD exerts a more insidious and sustained deleterious effect than transient Aβ exposure (48). In contrast to the inventor's observation of no effect of the treatment on hippocampal Aβ load in 5×FAD mice, a recent study did report that MK0677 treatment lowered neocortical Aβ plaques in young 5×FAD mice (44). A higher dose of MK0677 used in that study in comparison to ours may partially explain this discrepancy in the effect of Ghsr1α activation on Aβ load. In addition, a previous study from this group found that systemic ghrelin treatment did not affect hippocampal Aβ load in 5×FAD mice (32). The inventor cannot rule out that MK0677 may affect Aβ production and/or clearance in a dose-dependent manner. This idea requires further investigation, taking into account the potential systemic effects on body weight and glucose regulation of high doses of this drug, since obesity is a risk for AD (49).

GHSR1α and DRD1 are abundantly co-expressed in the hippocampus and are believed to serve important roles in hippocampal function (19, 50). Kern and colleagues have determined a mechanism that links GHSR1α and DRD1 in the regulation of hippocampal synaptic function. They found that activated GHSR1α shifts DRD1 from a Gas to a Gaq state via the formation of GHSR1α/DRD1 heterodimers, which allows dopamine to activate hippocampal synaptic activity-related Ca²⁺ signaling (8). This pivotal role of the GHSR1α/DRD1 heterodimer in hippocampal synaptic physiology reinforces considering co-activation of GHSR1α and DRD1 for restoring synaptic defects. A further relevant finding from this study is that co-activation of Ghsr1α/Drd1 protects Ghsr1α from Aβ toxicity. The inventor proposes that this resistance to AP is conferred through a Ghsr1α conformational change that arises via its interaction with Drd1 . This observation provides another potential explanation for why using a GHSR1α agonist in isolation lacked clinical efficacy. In support of this hypothesis, previous studies revealed that simultaneous use of agonists of different GPCR family members can induce allosteric interactions and alterations in functional properties (51, 52). Moreover, although the inventor did not observe DRD1 alterations in AD hippocampi, he cannot conclude that DRD1 function is intact in AD. In addition to the observation that DRD1 B2 allele is an AD risk factor (53), previous studies showed damage in the locus coeruleus in patients with AD (54). Because tyrosine-hydroxylase-expressing neurons in the locus coeruleus project to the hippocampus, which enhances synaptic activity and hippocampus-related memory via D1-type dopamine receptors (55). It is therefore possible that dopaminergic input to the hippocampus is altered in AD. In this regard, the impaired regulation of DRD1 by GHSR1α in AD-related conditions may also result from insufficient supply of dopamine, DRD1′s natural ligand, in the hippocampus, which could be mitigated by the supplementation of DRD1 agonist. Lastly, in this study, the inventor used young 5×FAD mice, which have relatively mild synaptic lesions and no hippocampal neuron loss (13). Whether MK0677/SKF81297 benefits older 5×FAD mice with more pervasive hippocampal lesions remains untested and need further investigation. Nevertheless, this proof-of-concept study shows the potential protective effects of this AD dual-GPCR-agonist intervention. MK0677 is approved by Food and Drug Administration (FDA), and although SKF81297 is not, other DRD1 agonists including levodopa and pergolide are clinically available for clinical use (56).

Another question related to the protective effects of the treatment merits discussion is the role of neurogenesis. A previous study suggested that ghrelin attenuates hippocampal pathology in 5×FAD mice by potentiating hippocampal neurogenesis (32). Similarly, in this study the inventor found that co-activation of Ghsr1α and Drd1 promotes neurogenesis in the dentate gyms of mixture treated 5×FAD mice. The neurogenic effect of GHSR1α has been linked to its role in hippocampal energy metabolism (57) . However, the impact of altered neurogenesis on cognitive impairments in AD and whether neurogenesis can correct synaptic and brain network injury remained unclear (58).

Previous studies directly attribute hippocampal synaptic injury that observed in AD to Aβ toxicity and/or tauopathy (59). Although the current study explored Ghsr1α defects in the context of an Aβ-rich environment, potential broader effects of Ghsr1α deregulation on hippocampal metabolic processes and calcium signaling should not be overlooked. It is well-documented that alterations in metabolic hormones such as ghrelin, leptin, and insulin can affect feeding behavior and nutrient availability, culminating in alterations of brain energy homeostasis and synapse remodeling (19, 60). Therefore, ghrelin system perturbations could cause brain and systemic metabolic deregulation, which is strongly associated with deficits in synaptic activity and hippocampus-dependent memory in both aging and AD (19). Moreover, the hypothalamus, a target of many metabolic hormones, plays a crucial role in maintaining brain metabolic homeostasis and hippocampal synaptic physiology (61). Hypothalamic pathology occurs in patients with AD (62). Therefore, given GHSR1α's role in hypothalamic function (63), it is possible that GHSR1α deregulation may affect hypothalamic function and indirectly drive hippocampal damage. The inventor cannot rule out the possibility that the treatment-derived neuroprotection in this study may at least in part reflect improvements in hippocampal energy metabolism that arises secondary to effects on hypothalamic GHSR1α. Moreover, in view of the influence of GHSR1α on DRD1-mediated Ca²⁺ signaling pathway related to hippocampal synaptic plasticity (8), GHSR1α deregulation may also represent a mechanism of calcium signaling-associated selective neuronal vulnerability (SNV) of AD hippocampi (64). In this context, altered hypothalamic-hippocampal connectivity and perturbed calcium signaling at synapses that result from GHSR1α dysfunction might act as critical Aβ-independent metabolic and calcium-related mechanisms of hippocampal synaptic failure in AD.

Herein, the potential protective effects for AD therapy that result from the combination of two GPCR agonists are indicated, thus providing a novel strategy of drug development for the prevention and treatment of AD. Importantly, MK0677 is FDA-approved, and in addition to the potential of SKF81297 for clinical trials, several less-specific DRD1 agonists, including levodopa and pergolide, have been used in clinical settings for years⁶¹.

In summary, a novel mechanism of selective hippocampal pathology through GHSR1α deregulation and an innovative strategy for AD therapy is presented. While other factors may contribute to GHSR1α dysfunction in AD, the most parsimonious interpretation of these findings is that Aβ-induced GHSR1α deregulation constitutes a primary AD pathology and this may have important therapeutic implications for the treatment of AD.

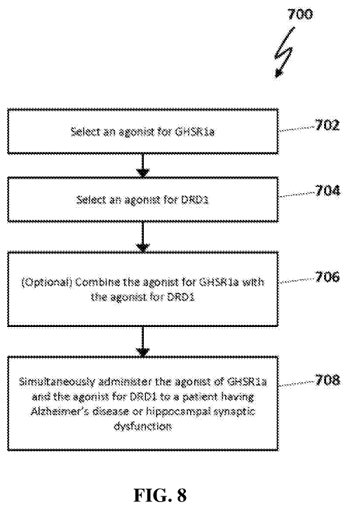

A method 700 according to an embodiment of the invention is described in FIG. 8. At step 702, an agonist for GHSR1α is selected. The agonist for GHSR1α is preferably selected from a list of agonists shown in Table 1. At step 704, an agonist for DRD1 is selected. The agonist for DRD1 is preferably selected from a list of agonists shown in Table 2. At step 708, the agonist for GHSR1α and the agonist for DRD1 are simultaneously administered to a patient having Alzheimer's disease or hippocampal synaptic dysfunction. In a preferred embodiment, the agonist for GHSR1α is MK0677 and the agonist for DRD1 is SKF81297.

TABLE 1
GHSR agonists
Adenosine
Alexamorelin
Anamorelin
Capromorelin
CP-464709
Cortistatin-14
Examorelin (hexarelin)
Ghrelin (lenomorelin)
GHRP-1
GHRP-3
GHRP-4
GHRP-5
GHRP-6
Ibutamoren (MK-677)
Ipamorelin
L-692,585
LY-426410
LY-444711
Macimorelin
Pralmorelin (GHRP-2)
Relamorelin
SM-130,686
Tabimorelin
Ulimorelin

TABLE 2
DRD1 agonists
dopamine
A-86929
Dihydrexidine
Dinapsoline
Dinoxyline
Doxanthrine
SKF-81297 (200-fold selectivity for D1)
SKF-82958
SKF-38393
Fenoldopam
6-Br-APB
Stepholidine
A-68930
A-77636
CY-208,243
SKF-89145
SKF-89626
7,8-Dihydroxy-5-phenyl-octahydrobenzo[h]isoquinoline
Cabergoline
Pergolide
levodopa
carbidopa

In step 708 the agonist for GHSR1α and the agonist for DRD1, which are drugs, may be administered in separate form, such as a pill form. However, the drugs must be administered at the same time, preferably in a prescribed proportion. In a preferred embodiment, the two types of agonists should be mixed in an optimized ratio to achieve the best efficacy and minimize potential side-effects. When the agonist for GHSR1α is MK0677 and the agonist for DRD1 is SKF81297, the preferred proportion of MK0677 to SKF81297 is 1:1.5 by weight.

Optionally, at step 706, the agonist for GHSR1α and the agonist for DRD1 are combined into a fixed-dose combination (FDC) product in a single dosage form for manufacturing and distributing. In a preferred embodiment, the FDC product contains the agonist for GHSR1α and the agonist for DRD1 in a prescribed proportion. Preferably, the two types of agonists should be combined in the FDC in an optimized ratio to achieve the best efficacy and minimize potential side-effects. In a preferred embodiment, the FDC is a combination of MK0677 with SKF81297, preferably in the ratio of 1:1.5 by weight.

### Materials and Methods

Study design. The objective of this study was to determine the role of GHSR1α in AD pathology and develop a strategy for preventing AD phenotype in a mouse model. Hippocampal tissues from patients with AD and non-AD controls were analyzed to determine GHSR1α expression, Aβ/GHSR1α interaction and GHSR1α/DRD1 complexes. The transgenic 5×FAD mouse model was used to mimic AD amyloidopathy (28). Ghsr null (20) and Ghsr null/5×FAD mice were used to explore the role of GHSR1α deficiency in an AD-like environment. To mimic mild cognitive impairment (MCI) and later stage AD respectively, the studies were performed using 4 month- and 9 month-old mice. Both male and female mice were used. The investigators performing the experiments did not allocate the mice.

For all the experiments, sample sizes were determined by the inventor's previous data, prior literature, and power calculation to ensure sufficient sample sizes to allow the detection of statistically significant differences. Sample exclusion was not permitted. The number of unique replicates for each experiment is specified in the figure legends. Mice were randomized by genotype and gender during behavioral testing. For the behavioral, electrophysiological, pathological, and Duolink proximity ligation assays, experimenters were blinded during data acquisition and unblinded for data analysis.

Statistical analysis. Statistical comparisons were performed using GraphPad Prism 5 software. One-way or two-way ANOVA followed by Bonferroni post hoc analysis, or unpaired two-way Student's t test were applied in data analysis. Pearson's correlation coefficient was used for correlation testing. Numbers of replicates and P values are stated in each figure legend. All data were expressed as the mean±s.e.m. except for the box plots which were shown as maximum, median and minimum. Significance was concluded when the P value was less than 0.05. Significance was indicated by symbols including * (P<0.05), ** (P<0.010), *** (P<0.001), # (P<0.001), t (P<0.001). NS (not significant) denotes P>0.05.

Human samples. Frozen postmortem hippocampal tissues and paraffin-embedded hippocampal slices were requested from the University of Texas (UT) Southwestern Medical Center ADC Neuropathology Core supported by ADC grant (AG12300) under a protocol approved by The UT Southwestern Medical Center as well as the University of Kansas (KU) Alzheimer's disease center Neuropathology Core under a protocol approved by KU Medical Center supported by NIH GRANT (P30 AG035982). Informed consent was collected from all subjects and the study adhered to the Declaration of Helsinki principles.

Mice. Animal studies were approved and performed under the guidelines of the University of Texas at Dallas (UTD) Institutional Animal Care and Use Committee (IACUC) and National Institutes of Health (NIH). 5×FAD mice (B6SJL-Tg (APPSwF1Lon, PSEN1*M146L*L286V) 6799Vas/Mmjax) (28) were originally obtained from Jackson Laboratory. Ghsr null mice on a pure C57BL/6N genetic background (20) from a colony maintained at UT Southwestern Medical Center were crossed with 5×FAD mice to generate litters including non-transgenic (non-Tg), Ghsr null, 5×FAD, and Ghsr null/5×FAD mice. Genotypes of animals were confirmed using PCR and/or amyloid plaques staining. The number of mice was determined by the inventor's previous data and power calculation to ensure that the minimal number of mice as required were used in the experiments.

Antibody validation. For accurate and reproducible results, the inventor validated all antibodies used in the current study as previously described (65). Most antibodies passed the validation; however, all commercially available antibodies against GHSR1α or DRD1 performed poorly in immunoblotting. Further antibody validation using non-Ghsr1α expressing mouse tissues and cells as well as non-Drd1 expressing cells as critical negative controls and GHSR1α- and DRD1-expressing tissues and cells as critical positive controls showed that anti-GHSR1α from Santa Cruz Biotechnology (#sc-10359) and anti-DRD1 from Abcam (#ab81296) exhibited specific and reproducible results in immunostaining and membrane blotting. The results suggest that the above mentioned anti- GHSR1α and anti-DRD1 antibodies only recognize antigens preserved in their natural structures in brain slices and isolated membranes. Therefore, in the current study the inventor conducted immunostaining and/or membrane blotting to reflect the expression of GHSR1α and DRD1, as well as membrane-incorporated GHSR1α and DRD1, respectively.

Oligomeric Aβ preparation. Aβ42 peptide (GenicBio, A-43-T-1000) was diluted in 1,1,1,3,3,3-hexafluoro-2-propanol (Sigma-Aldrich) to 1 mM. After centrifugation, the clear solution was then aliquoted in microcentrifuge tubes, and it was dried overnight in the fume hood. Peptide film was diluted in DMSO to 5 mM and sonicated for 10 minutes in bath sonicator. The peptide solution was resuspended in cold HAM′S F-12 (Sigma-Aldrich) to 100 μM and immediately vortexed for 30 seconds. The solution was then incubated at 4° C. for 24 hours to prepare oligomeric Aβ42.

Duolink In Situ Proximity Ligation Assay (PLA). Protein interactions between GHSR1α/DRD1 and Aβ3/GHSR1α, respectively, in human/mouse brain slices, cultured hippocampal neurons, and transfected HEK 293T cells were detected using Duolink In Situ PLA detection kits (Sigma-Aldrich, #DUO92008, #DUO92012), following the manufacturer's instruction. The following primary antibodies were used: goat polyclonal anti-GHSR1α (Santa Cruz Biotechnology, #sc-10359, 1:100), rabbit polyclonal anti-DRD1 (Abcam, #ab81296, 1:200), rabbit polyclonal anti-β-amyloid (CST, #8243, 1:1,000), mouse monoclonal anti-β-amyloid (CST, #15126, 1:1,000). Mouse anti-FLAG-tag (Thermo Fisher Scientific, #MA1-91878, 1:400) was used to recognize Ghsr1α-333 FLAG and its mutants in transiently transfected HEK 293T cells. The following Duolink in Situ PLA Probes were used: anti-Rabbit PLUS (Sigma-Aldrich, #DUO92002), anti-Goat MINUS (Sigma-Aldrich, #DUO92006), and anti-Mouse MINUS (Sigma-Aldrich, #DUO92004). Images were collected on a Nikon confocal microscope or Olympus upright microscope. PLA analysis was referred to previous report (66 67). NIH Image J was used for PLA signal quantification. The threshold was adjusted to attain the best visualization of the PLA-positive signal. The numbers and intensity of PLA-positive dots were counted and analyzed using NIH Image J “analyze particles” plug-in. The number of PLA-positive dots were counted and divided by the area to represent specific protein interaction. For mouse brain slice, HEK 293T cells and cultured hippocampal neurons (Aβ/GHSR1α only for cultured hippocampal neurons), due to the dense signal, the mean intensity (defined as arbitrary unit) of positive fluorescence signals was quantified instead of number of dots.

Immunocytochemistry. Mouse brains were dissected and immediately fixed in 4% paraformaldehyde (PFA) (Sigma-Aldrich) for 24-26 hours at 4° C. The frozen tissue sections were prepared as previously described (68). Primary cultured neurons on Lab-Tek chamber slides were fixed in 4% PFA for 30 minutes at 37° C. The slices or neurons were blocked with blocking buffer (5% goat or donkey serum, 0.3% Triton X-100 in PBS, pH 7.4) for 1 hr, then incubated with primary antibodies at room temperature overnight. Dilutions of antibodies were as follows: goat-anti-GHSR1α (Santa Cruz Biotechnology, #sc-10359, 1:100), rabbit-anti-DRD1 (Abcam, #ab81296, 1:200), rabbit-anti-β-amyloid (CST, #8243,1:1,000) for Aβ deposition detection, mouse-anti-β-amyloid (CST, #15126,1:1,000), rabbit-anti-PSD 95 (CST, #3450, 1:400), guinea pig-anti-vGLUT1 (Synaptic system, #135304, 1:400), mouse-anti-MAP2 (Sigma-Aldrich, #M4403, 1:300), rabbit-anti-MAP2 (CST, #4542, 1:600), mouse-anti-NeuN (Millipore, #MAB377, 1:600), mouse-anti-FLAG-tag (Thermo Fisher Scientific, #MA1-91878, 1:400), rabbit-anti-HA-tag (CST, #3724S, 1:400), mouse-anti-HA-tag (CST, #2367, 1:400), mouse-anti-Doublecortin (Santa Cruz Biotechnology, #sc271390, 1:100). After washing with PBS, the slices or neurons were probed with appropriate cross-adsorbed secondary antibodies conjugated to Alexa Fluor 488, Alexa Fluor 594 or Alexa Fluor 647 (Thermo Fisher Scientific, 1:500). Images were collected on a Nikon confocal microscope or inverted fluorescence microscope. NIS element software was used for image analysis. “Objective Count” dialog was used for intensity measurement. Positive staining was identified in the image using an intensity threshold set at the mean pixel intensity for the entire image plus the standard deviation of the pixel intensity. Total fluorescence intensity was taken as the sum of all intensity in these objects, which then was divided by the area of objects to calculate the average fluorescence intensity per area. For synapse density counting, vGLUT1 and PSD95 stained channels were saved as two binary layers which were overlapped by using “AND” operation in “binary operation” dialog of NIS element software. The overlapped dots represent synapses. For images from brain slice, average synapse number per volume was calculated. For images from primary neurons, the synapse number per length of dendritic segment was calculated. All confocal images were converted to 3 dimensional images by using NIS element software “3D reconstruction” module for analysis.

Plasmid construction. Mouse full length Ghsr1α cDNA (NCBI Gene ID: 208188) was purchased from OriGene (MR226073). Drd1 cDNA (NCBI Gene ID: 13488) was isolated by PCR from mouse brain cDNA. Ghsr1α cDNA and C terminal 333 FLAG tag, or Drd1 cDNA and C terminal HA tag, were subcloned into pcDNA3 to generate pcDNA3-Ghsr1α-333 FLAG and pcDNA3-Drd1-HA. To screen the Aβ binding site on Ghsr1α, seven deleted forms of Ghsr1α were generated (FIG. S3). The integrity of all constructs generated by PCR and subcloning was confirmed by nucleotide sequencing.

HEK 293T cell culture and transfection. HEK 293T cells (ATCC) were cultured in Dulbecco's Modified Eagle's Medium (DMEM, Sigma-Aldrich) with 10% FBS (Sigma-Aldrich) and penicillin—streptomycin (Thermo Fisher Scientific) at 37° C. and 5% CO₂.Trypsinized cells were seeded on poly-D-lysine-coated culture dishes for Co-IP, Lab-Tek chamber slides (Nunc, #177445) for immunostaining or black culture plates (Corning, 3603 or 4580) for FlAsH-Fret assay. Calcium-phosphate method was used for plasmid transfection on HEK 293T cells.

Co-immunoprecipitation assay (Co-IP). pcDNA3-Ghsr1α-333 FLAG or its mutants transfected HEK 293T cells were collected in IP buffer containing 50 mM Tris-HC1 (Fisher Scientific), pH 7.4, 150 mM NaCl (Fisher Scientific), 1 mM EDTA (Fisher Scientific), 0.1% NP-40 (Fisher Scientific) and protease inhibitor cocktail (Calbiochem, set V, EDTA free). After three freeze-thaw cycles, the cell lysates were centrifuged at 12,000×g for 5 minutes at 4° C. The supernatants were incubated with mouse anti-FLAG (Thermo Fisher Scientific, # MA1-91878, 0.5 μg IgG per 100m protein ratio) at 4° C. overnight, followed by an incubation with pre-washed protein A/G agarose beads (Pierce) for 2 hours at room temperature. Non-immune mouse IgG was used as negative control. After 6 times of washing with IP buffer, the protein complexes were eluted by boiling in 1×NuPAGE LDS Sample Buffer (Thermo Fisher Scientific) and subjected to immunoblotting with antibody to Aβ (CST, #8243, 1: 1,000). Anti-FLAG (Thermo Fisher Scientific, # MA1-91878, 1:2,000) was used for input detection of Ghsr1α-333 FLAG and its mutants.

Drd1 and Aβ interaction was tested from pcDNA3-Drd1-HA transfected HEK 293T cell lysates. Mouse anti-HA (CST, #2367, 1:400) was used for Drd1-HA immunoprecipitation. Rabbit anti-HA (CST, #3724S, 1:400) was used for Drd1-HA immunoblotting.

FlAsH-based FRET. The cDNA encoding the enhanced CFP (ECFP) was fused to C terminus of mouse Ghsr1α cDNA (Gene ID: 208188) or mouse Drd1 cDNA (Gene ID: 13488). The CCPGCC motif which has higher binding affinity for FlAsH was inserted in the third intracellular loop between Va1247 and Gly248 of Ghsr1α cDNA or between Thr245 and Gly246 of Drd1 cDNA. The cDNAs were cloned into pcDNA3 to generate construct pcDNA3-Ghsr1α^{FlAsH/ECFP} and pcDNA3-Drd1^{FlAsH/ECFP} using in-fusion system from Takara and verified by sequencing. HEK 293T cells were cultured on black 96 well plate with clear bottom (Corning) and transfected using calcium phosphate method. FlAsH labeling was performed as previously described (18). 50 μM MK0677 or 100 μM SKF81297 and/or 2 μM Aβ were used as the treatments. JMV2959 at a concentration of 50 μM was used to offset the effect of MK0677. Fluorescence signals were monitored on microplate reader (BioTek Cytation 5) before and after the treatments. The following excitation/emission wavelengths were used: 425±20 nm/475±20 nm for ECFP; 500±20 nm/535±20 nm for FlAsH; 425±20 nm/535±20 nm for FRET between ECFP and FlAsH. Area scanning read mode was used to correct artificial inaccuracies. The FRET ratio (F_{FlAsH}/F_{ECFP}) was calculated according to equation as previously reported (69):

Ratio (F_{FlAsH}/F_{ECFP})=(F_{FlAsH}^{ex425/em535}−α33 F_{ECFP}^{ex425/em475}−b×F_{FlAsH}^{ex500/em535})/F_{ECFP}^{ex425/em475}

In this equation, the leakage from ECFP into 535 nm channel (F_{ECFP}^{ex425/em475}) and from the direct FlAsH excitation by light at 425 (F_{FlAsH}^{ex500/em535}) were subtracted from the FlAsH signal after ECFP excitation (F_{FlAsH}^{ex425/em535}). The terms a and b are correction factors for the two leakages mentioned above. a is the ratio of the direct emission of donor, ECFP at 535 nm, to its emission at 475 nm when excited at 425 nm which was calculated with cells transfected with pcDNA3-Ghsr1α^{FlAsH/ECFP} or pcDNA3-Drd1^{FlAsH/ECFP} only. b is the ratio of the direct emission of FlAsH at 535 nm when excited at 425 nm to its emission at 535 nm when excited at 500 nm which was calculated with FlAsH labeled untransfected cells.

For FRET assay with Ghsr1α/Drd1 co-activation, the inventor generated pcDNA3-Ghsr1α^{FlAsH/ECFP}-T2A-Drd1-HA construct. Ghsr1α^{FlAsH/ECFP} and mouse Drd1-HA cDNA were cloned into pcDNA3 with T2A sequence (GSGEGRGSLLTCGDVEENPGP) inserted between those two cDNAs to express both proteins simultaneously. 50 μM MK0677 and 100 μM SKF81297 were added on HEK 293T cells at the same time to co-activate both Ghsr1α and Drd1 proteins in the presence or absence of Aβ (2 μM).

For cell imaging, HEK 293T cells were cultured on 96 well plate with cover glass bottom (Corning). The images were taken using an Olympus FV3000RS confocal microscope equipped with oil immersion 40× objective. ECFP was excited at 445 nm and images were taken with the factory setting for ECFP fluorescence (460 nm-500 nm). FlAsH was excited at 488 nm and images were taken with the factory setting for EYFP fluorescence (530 nm-580 nm). FRET between ECFP and FlAsH was excited at 445 nm and images were taken with the factory setting for EYFP fluorescence (530 nm-580 nm). The images on the same cell areas were taken before and after treatment. Image J software (NIH) was used to calculate FRET ratio (FlAsH^{exECFP/emFlAsH}/ECFP^{exFlAsH/emECFP}) and generate pseudocolor images.

Mouse behavioral test. Morris water maze test was performed as previously described in order to test changes in mice spatial learning and memory (70). Mice were allowed to acclimate to the testing environment at least 0.5 hr before tests. Mice were randomized by gender and genotypes to which the experimenter was blinded during the tests. In brief, mice were trained to find a hidden platform (20 cm diameter) in an open swimming pool (200 cm diameter) filled with 21° C. water. Four trials were performed each day for 12 days. Each trial started at a different position (NW, N, E, SE) while the platform was kept in a single location (SW). Each trial lasted 60 seconds, followed by 30 seconds during which mice were allowed to remain on the platform to give them an opportunity to memorize the location of the platform. After 12 days of training, mice were subjected to a probe test in which the platform was removed. The latency they needed to reach the platform or the number of times they passed the previous platform location were analyzed using HVS Image 2015 software (HVS Image) to present mice learning curves and probe results.

Primary hippocampal neuron culture. Hippocampal neuron cultures were prepared as previously described (15). In brief, mouse hippocampi were dissected from postnatal day 0-1 pups in cold HBSS. Cells were dissociated by using 0.025% trypsin at 37° C. for 15 minutes, followed by 10 times homogenization in ice cold DMEM. Dissociated cells were then passed through a 100 μm cell strainer (Corning) and centrifuged for 5 minutes at 210×g. The pellet was gently resuspended in neuron culture medium (Neurobasal A with 2% B27 supplement, 0.5 mM L-glutamine, Invitrogen) and plated on poly-D-lysine (Sigma-Aldrich) coated Lab-Tek chamber slides (Nunc, 177445) with appropriate densities.

Agonist treatment on primary cultured neurons. At 14 days in vitro (DIV14) hippocampal neurons were exposed to the synthetic growth hormone secretagogue receptor agonist MK0677 (Tocris, #5272, 1.5 μM) the dopamine D1-like receptor agonist SKF81297 (Sigma-Aldrich, #S143, 2 μM) or a mixture of MK0677 and SKF81297 for 5 minutes. The exposure was followed by immunostaining to examine the effects of Ghsr1α/Drd1 co-activation on Aβ induced synaptic loss or Ghsr1α/Drd1, Aβ/Ghsr1α interaction as described in previous sections.

Electrophysiology. For electrophysiological experiments, mice were anesthetized with isoflurane and decapitated. Brains were extracted and transverse sections (350 μm) of the hippocampus were cut on a vibratome (VT1200S, Leica) in ice-cold oxygenated (95% O₂, 5% CO₂) ACSF containing the following: 110 mM choline (Sigma-Aldrich), 25 mM NaHCO₃ (Fisher Scientific), 1.25 mM NaH₂PO₄ (Fisher Scientific), 2.5 mM KCl (Sigma-Aldrich), 7 mM MgCl₂ (Sigma-Aldrich), 0.5 mM CaCl₂ (Sigma-Aldrich), 10 mM dextrose (Fisher Scientific), 1.3 mM L-ascorbic acid (Fisher Scientific), and 2.4 mM sodium pyruvate (Sigma-Aldrich). Slices were incubated for at least 1 hr in normal recording ACSF consisting of: 126 mM NaCl (Fisher Scientific), 25 mM NaHCO₃, 1.25 mM NaH₂PO₄, 2.5 mM KCl, 1.3 mM MgCl₂, 2 mM CaCl₂, 10 mM dextrose, 2.4 mM sodium pyruvate, and 1.3 mM L-ascorbic acid, bubbled with 95% O₂/5% CO₂. Slices were allowed to rest for 30 minutes after being transferred to the recording chamber before recordings began. Recordings of local field potentials (LFPs) were performed on an Axon Multiclamp 700B amplifier (Molecular Devices), and data were acquired and analyzed using AxoGraph X (AxoGraph Scientific). A tungsten concentric bipolar microelectrode (World Precision Instruments), and a recording glass electrode (1.5 MS2) filled with recording ACSF, were placed approximately 200 μm apart in the Schaffer collateral-commissural pathway in the CA1 region of the hippocampus. Pulses were delivered in 30 seconds intervals. Before baseline recordings commenced, input-output curves were taken, using 25-150 μA stimulation currents in 25 μA steps. Three sweeps were sampled at each stimulation intensity and averaged to represent the voltage response at each step. Recordings of baseline responses lasted for at least 40 minutes. Stimulation intensity was set to approximately 40% of the minimum intensity required to evoke the maximum response (based on the input-output curve). The stimulation intensity was kept consistent throughout the duration of the experiment. All drugs (1.5 μM MK0677 and 2 μM SKF81297) were bath applied for 20 minutes prior to long-term potentiation (LTP) induction. LTP was induced using a theta burst stimulation (TBS) protocol consisting of 10 bursts (5 pulses at 100 Hz) repeated at 5 Hz delivered two times 30 seconds apart. After LTP induction, LFPs were recorded for an additional 60 minutes. The slope of the field excitatory postsynaptic potential (fEPSP) was measured in Axograph scientific software and sweeps were averaged in bins of 5 for both the baseline and post LTP induction periods. Changes in LFPs are expressed as percentage change from the averaged baseline values.

Whole-cell voltage-clamp recordings were obtained at room temperature using oxygenated (95% O₂, 5% CO₂, pH 7.3) recording ACSF containing the following: 120 mM NaCl, 2.5 mM KCl, 1.25 mM NaH₂PO₄, 25 mM Na₂HCO₃, 10 mM dextrose, 2 mM CaCl₂, and 2 mM MgCl₂, 2.4 mM sodium pyruvate, and 1.3 mM L-ascorbic acid. Slices treated with MK0677/SKF81297 were incubated for at least 1 hr in ACSF containing 1.5 μM MK0677 and 2 μM SKF81297 prior to the start of recordings. For voltage-clamp recordings, electrodes (WPI; 3-5 MΩ open tip resistance) were filled with the following: 130 mM CsCl (Sigma-Aldrich), 20 mM tetraethylammonium chloride (Sigma-Aldrich), 10 mM HEPES (Sigma-Aldrich), 2 mM MgCl₂, 0.5 mM EGTA (Sigma-Aldrich), 4 mM Na₂-ATP (Sigma-Aldrich), 0.3 mM Lithium-GTP (Sigma-Aldrich), 14 mM phosphocreatine (Sigma-Aldrich), and 2 mM QX-314 bromide (Tocris Bioscience) and brought to a pH of 7.2 with CsOH (Fischer Scientific). AMPA-mediated miniature excitatory postsynaptic currents (AMPA-mEPSCs) were pharmacologically isolated by adding 75 μM picrotoxin (Sigma-Aldrich), 1 μM tetrodotoxin (Alomone Labs) and 10 μM CPP ((±)-3-(2-Carboxypiperazin-4-yl)propyl-1-phosphonic acid, Sigma-Aldrich) to the recording ACSF. Access resistance was monitored throughout the recording, and a 20% change was deemed acceptable. The frequency and amplitude of AMPA-mEPSCs were measured from 200 seconds of continuous recording using MiniAnalysis (Synaptosoft) with a threshold set at two times the RMS baseline noise.

Agonist treatment on mice. 3 months-old non-Tg and 5×FAD mice received daily intraperitoneal (i.p.) injections of saline (sterilized 0.9% NaCl), or a combination of MK0677 (Tocris, #5272, a dose of 1 or 3 mg/kg) and SKF81297 (Sigma-Aldrich, #S143, a dose of 1.5 or 4.5 mg/kg) diluted in saline for one month. After the treatment the mice were subjected to behavioral test, then euthanized for tissue collection.

Cell membrane isolation and membrane blotting. Human or mouse hippocampal cell membranes were extracted using a previously published protocol (71). In brief, hippocampi were homogenized and incubated in ice-cold isolation buffer containing 40 mM Tris-HCl, pH 7.4, 1 mM MgCl₂ (Fisher Scientific), 0.15 U/μl benzonase (EMD Millipore) for 10 minutes. Three times 12,000g×10 minutes centrifugation were performed to isolate and wash cell membrane. Purified hippocampal cell membrane were then fixed in 4% PFA for 0.5 hrs followed by 1 hr blocking (5% donkey/goat serum, 0.3% Trition-X-100, PBS, pH 7.4). Membranes were incubated overnight in primary anti-GHSR1α or anti-DRD1 antibody at 4° C., followed by 1 hr incubation with anti-goat or anti-rabbit HRP-conjugated secondary antibody at room temperature. In order to remove non-specific binding of primary and secondary antibodies membranes were washed in PBST (PBS containing 0.05% Tween-20) for three times followed by centrifugation at 16,500×g for 20 minutes. Cell membrane protein were then extracted by using urea buffer containing 50 mM Tris-HCl, pH 6.8, 8 M urea (Fisher Scientific), 2% SDS (Fisher Scientific), 10% glycerol (Fisher Scientific). Cell membrane extracts were loaded onto nitrocellulose membrane (Bio-Rad). The nitrocellulose membrane was dried, then subjected to imaging immediately by using Bio-Rad Chemidoc Imaging System. The membrane was reprobed with mouse anti-β-III-tubulin (Proteintech, #66240, 1:1,000) to normalize protein expression.

Mouse serum total ghrelin ELISA. Mice were fasted for 8 hours before blood collection. Rat/Mouse total Ghrelin ELISA kits (Millipore, EZRGRT-91K) were used for serum total ghrelin measurement following the manufacturer's instruction. Data were collected on a microplate reader (BIOTEK) and the concentration of ghrelin was calculated (ng/ml).

Postsynaptic density isolation. Hippocampal postsynaptic densities (PSD) were prepared based on a published protocol (72). In brief, the hippocampi were dissected and homogenized in ice-cold homogenizing buffer (25 mM Tris-HCl, 0.32 M sucrose (Fisher Scientific), 1 mM phenylmethylsulfonyl fluoride (PMSF, Fisher Scientific), 1 mM EDTA, 1 mM EGTA (Fisher Scientific), 10 mM Na₃VO₄ (Fisher Scientific), 25 mM NaF (Fisher Scientific), 10 mM Na₄P₂O, (Fisher Scientific) and protease inhibitor cocktail, pH 7.5) with a Dounce homogenizer (Wheaton). The resultant homogenates were centrifuged at 1,000×g for 10 minutes to remove cell debris and nuclei. The supernatants were then centrifuged at 12,000×g at 4° C. for 15 minutes. Then the pellets (crude synaptosome) were resuspended in homogenizing buffer containing 1% Triton X-100 and 300 mM NaCl on ice for 30 minutes. After centrifugation at 16,000 g for 30 minutes the pellets were obtained as PSD (post synaptic density) fraction. Purified hippocampal PSD samples were resuspended in urea buffer for immunoblotting.

Immunoblotting. Samples were prepared in 1×NuPAGE LDS Sample Buffer or urea buffer as needed. Proteins were separated in 10% or 12% Bis-Tris Gel (Thermo Fisher Scientific) and then transferred to PVDF membrane (Bio-Rad). After blocking in 5% non-fat milk (Labscientific Inc) in TBS buffer (20 mM Tris-HC1, 150 mM NaCl, pH 7.6) for 1 hr at room temperature, the membranes were probed with appropriate primary antibodies overnight at 4° C. followed by incubation with the corresponding secondary antibody for 1 hr at room temperature. The following antibodies were used: rabbit monoclonal anti-Phospho-CaMKII (Thr286) (CST, #12716, 1:2,000), mouse monoclonal anti-CaMKII-α (CST, #50049, 1:5,000), mouse monoclonal anti-β-III-tubulin (Proteintech, #66240, 1:5,000), mouse monoclonal anti-Phospho-Tau (Ser202, Thr205) (Thermo Fisher, #MN1020, 1:1,000), mouse monoclonal anti-Phospho-Tau (Ser396) (CST, #9632, 1:5,000), rabbit monoclonal anti-Phospho-Tau (Ser404) (CST, #20194, 1:5,000), mouse monoclonal anti-T-Tau (Tau46) (CST, #4019, 1:1,000), mouse anti-β actin (Sigma-Aldrich, #5441, 1:10,000), mouse anti-amyloid precursor protein (BioLegend, #803002, 1:2000), goat anti-mouse IgG HRP conjugated and goat anti-rabbit IgG HRP conjugated secondary antibodies (Thermo Fisher Scientific, #31430 and 31460, 1:2,000-8,000). Images were collected on a Bio-Rad Chemidoc Imaging System. Image J software (NIH) was used for analysis.

Immunohistochemistry and H&E staining analysis. Paraffin-embedded human hippocampal sections were deparaffinized in xylene and rehydrated in a graded ethanol series. Heat induced antigen retrieval was performed in boiling citrate buffer (pH 6.0) for 15 minutes. Endogenous peroxidase activity was blocked with 0.3% hydrogen peroxide. Slides were blocked in PBS containing 5% goat or donkey serum (Sigma-Aldrich) and 0.3% Triton X-100 (Fisher Scientific) for 1 hr, then incubated with following primary antibodies at room temperature overnight: anti-GHSR1α (Santa Cruz Biotechnology, #sc-10359, 1:100) or anti-DRD1 (Abcam, #ab81296, 1:200) followed by 1 hr room temperature incubation of Biotin-conjugated secondary antibody (anti-goat from Invitrogen, #A16003, 1:500; anti-rabbit from Sigma-Aldrich, #B8895, 1:500) and ExtrAvidin—peroxidase (Sigma-Aldrich, #E2886, 1:500). Signal was developed using DAB (Sigma-Aldrich, #D4168). Hematoxylin (Sigma-Aldrich, MHS-16) was used for nuclear counterstain. Images were collected on Olympus upright microscope, mean intensity of the DAB signal were analyzed using Image J software (NIH) to represent human hippocampal GHSR1α and DRD1 expression.

Mice brain, kidney and liver sections were freshly dissected and fixed in 4% PFA overnight at 4° C. then proceeded to frozen tissue sectioning. Hematoxylin-Eosin (H&E) staining were performed following the commercial protocol. Briefly, slices are air dried overnight then proceeded to rehydration with 100%, 95%, 80%, 75% and 50% ethanol. Slices were stained with hematoxylin (Sigma-Aldrich, MHS-16) for 10 minutes followed by 5 minutes tap water rinsing. Next, the inventor put the slices into eosin (Sigma-Aldrich, HE110316) for 1 minute then dipped into ddH2O. Dehydration with reversed order of ethanol as rehydration were performed after the staining. Images were collected on Olympus upright microscope. Cell density were counted using Image J software (NIH).

Parenchymal amyloid plaques staining/Congo red staining. Parenchymal amyloid plaques were stained as previous described (73). The brain slices were dried overnight then rinsed in ddH₂O for 30 seconds. Afterwards, the brain slices were immersed in saturated NaCl solution (over saturated NaCl in 80% ethanol) with 10 mM NaOH for 20 min, followed by the immersion in 0.2% Congo red solution (0.2% Congo red in saturated NaCl solution, 10 mM NaOH) for 30 min. After the staining, the brain slices were quickly dipped in 90% ethanol for 8 times followed by 8× quick dip in 100% ethanol and then 3×5min′ s xylene incubation. Images were collected on Olympus upright microscope. The occupied area of Congo red-labelled Parenchymal amyloid plaques was analyzed using Image J software (NIH).

Intraneuronal amyloid β staining. PFA fixed frozen tissue were sectioned and proceeded to intraneuronal amyloid β staining as previously described (13). Briefly, mouse brain sections were blocked with 5% goat serum and 0.3% Triton X-100 for 1 hr. Slices were then probed with rabbit monoclonal anti-Aβ (1:500, Invitrogen, #700254) to detect intraneuronal amyloid β expression. Mouse monoclonal anti-β-III-tubulin (Proteintech, #66240, 1:1,000) was used to determine neurons and DAPI was used for nuclear staining. Images were collected on a Nikon confocal microscope. Aβ positive signals which were overlapped with β-III-tubulin were considered as intraneuronal amyloid β staining and its occupied area were analyzed by using “AND” operation in “binary operation” dialog of NIS element software. All confocal images were converted to 3 dimensional images by using NIS element software “3D reconstruction” module for analysis.

ELISA assay for soluble Aβ measurement. Aβ amounts in human or mouse hippocampal samples were measured by using human Aβ40 and Aβ42 ELISA kits (Thermo Fisher Scientific, KHB3481 for Aβ40, KHB3441 for Aβ42) following the manufacturer's instructions. Tissues were homogenized thoroughly with cold 5 M guanidine HCl/50 mM Tris HCl. The homogenates were incubated at room temperature for 4 hours. The samples were diluted with cold reaction buffer (Dulbecco's phosphate buffered saline with 5% BSA and 1× protease inhibitor cocktail) and centrifuged at 16,000×g for 20 minutes at 4° C. The supernatants were diluted with standard diluent buffer provided in the kit and quantified by ELISA kits. AP amounts were normalized to total protein content in the samples.

### REFERENCES FOR EXAMPLE 1

The following references, to the extent that they provide exemplary procedural or other details supplementary to those set forth herein, are specifically incorporated herein by reference.
1. S. W. Scheff, D. A. Price, F. A. Schmitt, E. J. Mufson, Hippocampal synaptic loss in early Alzheimer's disease and mild cognitive impairment. Neurobiol Aging 27, 1372-1384 (2006); published online EpubOct (10.1016/j.neurobiolaging.2005.09.012).
2. W. V. Graham, A. Bonito-Oliva, T. P. Sakmar, Update on Alzheimer's Disease Therapy and Prevention Strategies. Annu Rev Med 68, 413-430 (2017); published online EpubJan 14 (10.1146/annurev-med-042915- 103753).
3. B. K. W. Mani, A. K. Lopez Soto, E. J. Raingo, J. Lee, C. E. Perello, M. Andrews, Z. B. Zigman, J. M., Neuroanatomical characterization of a growth hormone secretagogue receptor-green fluorescent protein reporter mouse. J Comp Neurol 522, 3644-3666 (2014); published online EpubNov 1 (10.1002/cne.23627).
4. T. M. H. Hsu, J. D. Konanur, V. R. Noble, E. E. Suarez, A. N. Thai, J. Nakamoto, E. M. Kanoski, S. E., Hippocampus ghrelin signaling mediates appetite through lateral hypothalamic orexin pathways. eLife 4, (2015); published online EpubDec 15 (10.7554/eLife.11190).
5. B. K. Mani, S. Osborne-Lawrence, M. Mequinion, S. Lawrence, L. Gautron, Z. B. Andrews, J. M. Zigman, The role of ghrelin-responsive mediobasal hypothalamic neurons in mediating feeding responses to fasting. Mol Metab 6, 882-896 (2017); published online EpubAug (10.1016/j.molmet.2017.06.011).
6. S. E. Kanoski, S. M. Fortin, K. M. Ricks, H. J. Grill, Ghrelin signaling in the ventral hippocampus stimulates learned and motivational aspects of feeding via PI3K-Akt signaling. Biol Psychiatry 73, 915-923 (2013); published online EpubMay 1 (10.1016/j.biopsych.2012.07.002).
7. T. M. Hsu, E. E. Noble, D. J. Reiner, C. M. Liu, A. N. Suarez, V. R. Konanur, M. R. Hayes, S. E. Kanoski, Hippocampus ghrelin receptor signaling promotes socially-mediated learned food preference. Neuropharmacology 131, 487-496 (2018); published online EpubMar 15 (10.1016/j.neuropharm.2017.11.039).
8. A. Kern, M. Mavrikaki, C. Ullrich, R. Albarran-Zeckler, A. F. Brantley, R. G. Smith, Hippocampal Dopamine/DRD1 Signaling Dependent on the Ghrelin Receptor. Cell 163, 1176-1190 (2015); published online EpubNov 19 (10.1016/j.cell.2015.10.062).
9. A. Kern, C. Grande, R. G. Smith, Apo-Ghrelin Receptor (apo-GHSR1α) Regulates Dopamine Signaling in the Brain. Front Endocrinol (Lausanne) 5, 129 (2014)10.3389/fendo.2014.00129).
10. J. J. R. Sevigny, J. M. van Dyck, C. H. Peng, Y. Lines, C. R. Nessly, M. L. M. K. , Growth hormone secretagogue MK-677: no clinical effect on AD progression in a randomized trial. Neurology 71, 1702-1708 (2008); published online EpubNov 18 (10.1212/01.wnl.0000335163.88054.e7).
11. R. S. J. Seminara, C. Biswas, S. Kanwal, B. Iftikhar, W. Sakibuzzaman, M. Rutkofsky, I. H., The Neurocognitive Effects of Ghrelin-induced Signaling on the Hippocampus: A Promising Approach to Alzheimer's Disease. Cureus 10, e3285 (2018); published online EpubSep 11 (10.7759/cureus.3285).
12. X. Cao, M. Zhu, Y. He, W. Chu, Y. Du, H. Du, Increased Serum Acylated Ghrelin Levels in Patients with Mild Cognitive Impairment. J Alzheimers Dis 61, 545-552 (2018)10.3233/JAD-170721).
13. W. A. Eimer, R. Vassar, Neuron loss in the SXFAD mouse model of Alzheimer's disease correlates with intraneuronal Abeta42 accumulation and Caspase-3 activation. Mol Neurodegener 8, 2 (2013); published online EpubJan 14 (10.1186/1750-1326-8-2).
14. M. Manczak, M. J. Calkins, P. H. Reddy, Impaired mitochondrial dynamics and abnormal interaction of amyloid beta with mitochondrial protein Drp1 in neurons from patients with Alzheimer's disease: implications for neuronal damage. Hum Mol Genet 20, 2495-2509 (2011); published online EpubJul 1 (10.1093/hmg/ddr139).
15. S. J. Beck, L. Guo, A. Phensy, J. Tian, L. Wang, N. Tandon, E. Gauba, L. Lu, J. M. Pascual, S. Kroener, H. Du, Deregulation of mitochondrial F1FO-ATP synthase via OSCP in Alzheimer's disease. Nat Commun 7, 11483 (2016); published online EpubMay 6 (10.1038/ncomms11483).
16. M. Sadowski, J. Pankiewicz, H. Scholtzova, J. A. Ripellino, Y. Li, S. D. Schmidt, P. M. Mathews, J. D. Fryer, D. M. Holtzman, E. M. Sigurdsson, T. Wisniewski, A synthetic peptide blocking the apolipoprotein E/beta-amyloid binding mitigates beta-amyloid toxicity and fibril formation in vitro and reduces beta-amyloid plaques in transgenic mice. The American journal of pathology 165, 937-948 (2004); published online EpubSep (10.1016/50002-9440(10)63355-X).
17. M. I. Lutz, C. Schwaiger, B. Hochreiter, G. G. Kovacs, J. A. Schmid, Novel approach for accurate tissue-based protein colocalization and proximity microscopy. Scientific reports 7, 2668 (2017); published online EpubJun 1 (10.1038/s41598-017-02735-8).
18. C. Hoffmann, G. Gaietta, M. Bunemann, S. R. Adams, S. Oberdorff-Maass, B. Behr, J. P. Vilardaga, R. Y. Tsien, M. H. Ellisman, M. J. Lohse, A FlAsH-based FRET approach to determine G protein-coupled receptor activation in living cells. Nat Methods 2, 171-176 (2005); published online EpubMar (10.1038/nmeth742).
19. S. Diano, S. A. Farr, S. C. Benoit, E. C. McNay, I. da Silva, B. Horvath, F. S. Gaskin, N. Nonaka, L. B. Jaeger, W. A. Banks, J. E. Morley, S. Pinto, R. S. Sherwin, L. Xu, K. A. Yamada, M. W. Sleeman, M. H. Tschop, T. L. Horvath, Ghrelin controls hippocampal spine synapse density and memory performance. Nat Neurosci 9, 381-388 (2006); published online EpubMar (10.1038/nn1656).
20. Y. N. Jeffrey M. Zigman, Roberto Coppari, Nina Balthasar, Jacob N. Marcus, Charlotte E. Lee, Juli E. Jones, Amy E. Deysher, Amanda R. Waxman, Ryan D. White, Todd D. Williams, Jennifer L. Lachey, Randy J. Seeley, Bradford B. Lowell, Joel K. Elmquist, Mice lacking ghrelin receptors resist the development of diet-induced obesity. The Journal of Clinical Investigation 115, 3564-3572 (2005)10.1172/JCI26002).
21. B. K. O.-L. Mani, S. Mequinion, M. Lawrence, S. Gautron, L. Andrews, Z. B. Zigman, J. M., The role of ghrelin-responsive mediobasal hypothalamic neurons in mediating feeding responses to fasting. Mol Metab 6, 882-896 (2017); published online EpubAug (10.1016/j.molmet.2017.06.011).
22. G. A. H. Kerchner, C. P. Hammond-Rosenbluth, K. E. Xu, D. Rabinovici, G. D. Kelley, D. A. Vigneron, D. B. Nelson, S. J. Miller, B. L., Hippocampal CA1 apical neuropil atrophy in mild Alzheimer disease visualized with 7-T MRI. Neurology 75, 1381-1387 (2010); published online EpubOct 12 (10.1212/WNL.0b013e3181f736a1).
23. B. Huang, C. S. Yang, J. Wojton, N. J. Huang, C. Chen, E. J. Soderblom, L. Zhang, S. Kornbluth, Metabolic control of Ca2+/calmodulin-dependent protein kinase II (CaMKII)-mediated caspase-2 suppression by the B55beta/protein phosphatase 2A (PP2A). J Biol Chem 289, 35882-35890 (2014); published online EpubDec 26 (10.1074/jbc.M114.585844).
24. A. A. Patchett, R. P. Nargund, J. R. Tata, M. H. Chen, K. J. Barakat, D. B. Johnston, K. Cheng, W. W. Chan, B. Butler, G. Hickey, et al., Design and biological activities of L-163,191 (MK-0677): a potent, orally active growth hormone secretagogue. Proc Natl Acad Sci USA 92, 7001-7005 (1995); published online EpubJul 18 (10.1073/pnas.92.15.7001).
25. A. C. Paula-Lima, J. Brito-Moreira, S. T. Ferreira, Deregulation of excitatory neurotransmission underlying synapse failure in Alzheimer's disease. J Neurochem 126, 191-202 (2013); published online EpubJul (10.1111/jnc.12304).
26. S. L. Malkin, K. Kim, D. B. Tikhonov, A. V. Zaitsev, Properties of spontaneous and miniature excitatory postsynaptic currents of rat prefrontal cortex neurons. J Evol Biochem Phys 50, 506-514 (2014); published online EpubNov-Dec (10.1134/S0022093014060052).
27. L. Wang, L. Guo, L. Lu, H. Sun, M. Shao, S. J. Beck, L. Li, J. Ramachandran, Y. Du, H. Du, Synaptosomal Mitochondrial Dysfunction in 5×FAD Mouse Model of Alzheimer's Disease. PLoS One 11, e0150441 (2016)10.1371/journal.pone.0150441).
28. H. Oakley, S. L. Cole, S. Logan, E. Maus, P. Shao, J. Craft, A. Guillozet-Bongaarts, M. Ohno, J. Disterhoft, L. Van Eldik, R. Berry, R. Vassar, Intraneuronal beta-amyloid aggregates, neurodegeneration, and neuron loss in transgenic mice with five familial Alzheimer's disease mutations: potential factors in amyloid plaque formation. J Neurosci 26, 10129-10140 (2006); published online EpubOct 4 (10.1523/JNEUROSCI.1202-06.2006).
29. L. Lu, L. Guo, E. Gauba, J. Tian, L. Wang, N. Tandon, M. Shankar, S. J. Beck, Y. Du, H. Du, Transient Cerebral Ischemia Promotes Brain Mitochondrial Dysfunction and Exacerbates Cognitive Impairments in Young 5×FAD Mice. PLoS One 10, e0144068 (2015)10.1371/journal.pone.0144068).
30. H. Zheng, A. Bailey, M. H. Jiang, K. Honda, H. Y. Chen, M. E. Trumbauer, L. H. Van der Ploeg, J. M. Schaeffer, G. Leng, R. G. Smith, Somatostatin receptor subtype 2 knockout mice are refractory to growth hormone-negative feedback on arcuate neurons. Mol Endocrinol 11, 1709-1717 (1997); published online EpubOct (10.1210/mend.11.11.0016).
31. M. Xu, T. E. Koeltzow, G. T. Santiago, R. Moratalla, D. C. Cooper, X. T. Hu, N. M. White, A. M. Graybiel, F. J. White, S. Tonegawa, Dopamine D3 receptor mutant mice exhibit increased behavioral sensitivity to concurrent stimulation of D1 and D2 receptors. Neuron 19, 837-848 (1997); published online EpubOct (10.1016/S0896-6273(00)80965-4).
32. M. Moon, M. Y. Cha, I. Mook-Jung, Impaired hippocampal neurogenesis and its enhancement with ghrelin in 5×FAD mice. J Alzheimers Dis 41, 233-241 (2014)10.3233/JAD-132417).
33. S. Couillard-Despres, B. Winner, S. Schaubeck, R. Aigner, M. Vroemen, N. Weidner, U. Bogdahn, J. Winkler, H. G. Kuhn, L. Aigner, Doublecortin expression levels in adult brain reflect neurogenesis. Eur J Neurosci 21, 1-14 (2005); published online EpubJan (10.1111/j.1460-9568.2004.03813.x).
34. J. R. Menezes, M. B. Luskin, Expression of neuron-specific tubulin defines a novel population in the proliferative layers of the developing telencephalon. J Neurosci 14, 5399-5416 (1994); published online EpubSep (10.1523/JNEUROSCI.14-09-05399.1994).
35. L. F. C. Ribeiro, T. Santos, S. D. Benoist, M. van Leeuwen, J. F. Esteban, J. A. Carvalho, A. L., Ghrelin triggers the synaptic incorporation of AMPA receptors in the hippocampus. Proc Natl Acad Sci U S A 111, E149-158 (2014); published online EpubJan 7 (10.1073/pnas.1313798111).
36. M. D. Gahete, A. Rubio, J. Cordoba-Chacon, F. Gracia-Navarro, R. D. Kineman, J. Avila, R. M. Luque, J. P. Castano, Expression of the ghrelin and neurotensin systems is altered in the temporal lobe of Alzheimer's disease patients. J Alzheimers Dis 22, 819-828 (2010)10.3233/JAD-2010-100873).
37. G. Navarro, D. Aguinaga, E. Angelats, M. Medrano, E. Moreno, J. Mallol, A. Cortes, E. I. Canela, V. Casado, P. J. McCormick, C. Lluis, S. Ferre, A Significant Role of the Truncated Ghrelin Receptor GHS-R1b in Ghrelin-induced Signaling in Neurons. J Biol Chem 291, 13048-13062 (2016); published online EpubJun 17 (10.1074/jbc.M116.715144).
38. S. G. Gauthier, Alzheimer's disease: the benefits of early treatment. Eur J Neurol 12 Suppl 3, 11-16 (2005); published online EpubOct (10.1111/j.1468-1331.2005.01322.x).
39. M. B. Boada-Rovira, H. Cras, P. Baloyannis, S. Emre, M. Zhang, R. Bahra, R., Efficacy and safety of donepezil in patients with Alzheimer's disease: results of a global, multinational, clinical experience study. Drugs Aging 21, 43-53 (2004)10.2165/00002512-200421010-00004).
40. M. F. Egan, J. Kost, T. Voss, Y. Mukai, P. S. Aisen, J. L. Cummings, P. N. Tariot, B. Vellas, C. H. van Dyck, M. Boada, Y. Zhang, W. Li, C. Furtek, E. Mahoney, L. Harper Mozley, Y. Mo, C. Sur, D. Michelson, Randomized Trial of Verubecestat for Prodromal Alzheimer's Disease. N Engl J Med 380, 1408-1420 (2019); published online EpubApr 11 (10.1056/NEJMoa1812840).
41. S. Gauthier, H. H. Feldman, L. S. Schneider, G. K. Wilcock, G. B. Frisoni, J. H. Hardlund, H. J. Moebius, P. Bentham, K. A. Kook, D. J. Wischik, B. 0. Schelter, C. S. Davis, R. T. Staff, L. Bracoud, K. Shamsi, J. M. Storey, C. R. Harrington, C. M. Wischik, Efficacy and safety of tau-aggregation inhibitor therapy in patients with mild or moderate Alzheimer's disease: a randomised, controlled, double-blind, parallel-arm, phase 3 trial. Lancet 388, 2873-2884 (2016); published online EpubDec 10 (10.1016/S0140-6736(16)31275-2).
42. L. S. Honig, B. Vellas, M. Woodward, M. Boada, R. Bullock, M. Borrie, K. Hager, N. Andreasen, E. Scarpini, H. Liu-Seifert, M. Case, R. A. Dean, A. Hake, K. Sundell, V. Poole Hoffmann, C. Carlson, R. Khanna, M. Mintun, R. DeMattos, K. J. Selzler, E. Siemers, Trial of Solanezumab for Mild Dementia Due to Alzheimer's Disease. N Engl J Med 378, 321-330 (2018); published online EpubJan 25 (10.1056/NEJMoa1705971).
43. N. Kunath, T. van Groen, D. B. Allison, A. Kumar, M. Dozier-Sharpe, I. Kadish, Ghrelin agonist does not foster insulin resistance but improves cognition in an Alzheimer's disease mouse model. Scientific reports 5, 11452 (2015); published online EpubJun 19 (10.1038/srep11452).
44. Y. O. Jeong, S. J. Shin, J. Y. Park, B. K. Ku, J. S. Song, J. J. Kim, S. G. Jeon, S. M. Lee, M. Moon, MK-0677, a Ghrelin Agonist, Alleviates Amyloid Beta-Related Pathology in 5XFAD Mice, an Animal Model of Alzheimer's Disease. Int J Mol Sci 19, (2018); published online EpubJun 18 (10.3390/ijms19061800).
45. M. Eslami, B. Sadeghi, F. Goshadrou, Chronic ghrelin administration restores hippocampal long-term potentiation and ameliorates memory impairment in rat model of Alzheimer's disease. Hippocampus 28, 724-734 (2018); published online EpubOct (10.1002/hipo.23002).
46. E. J. Dhurandhar, D. B. Allison, T. van Groen, I. Kadish, Hunger in the absence of caloric restriction improves cognition and attenuates Alzheimer's disease pathology in a mouse model. PLoS One 8, e60437 (2013)10.1371/journal.pone.0060437).
47. V. V. Santos, R. Stark, D. Rial, H. B. Silva, J. A. Bayliss, M. B. Lemus, J. S. Davies, R. A. Cunha, R. D. Prediger, Z. B. Andrews, Acyl ghrelin improves cognition, synaptic plasticity deficits and neuroinflammation following amyloid beta (Abetal-40) administration in mice. J Neuroendocrinol 29, (2017); published online EpubMay (10.1111/jne.12476).
48. F. M. LaFerla, K. N. Green, Animal models of Alzheimer disease. Cold Spring Harb Perspect Med 2, (2012); published online EpubNov 1 (10.1101/cshperspect.a006320).
49. D. Gustafson, E. Rothenberg, K. Blennow, B. Steen, I. Skoog, An 18-year follow-up of overweight and risk of Alzheimer disease. Arch Intern Med 163, 1524-1528 (2003); published online EpubJul 14 (10.1001/archinte.163.13.1524).
50. C. M. O'Carroll, S. J. Martin, J. Sandin, B. Frenguelli, R. G. Morris, Dopaminergic modulation of the persistence of one-trial hippocampus-dependent memory. Learn Mem 13, 760-769 (2006); published online EpubNov-Dec (10.1101/1m.321006).
51. M. J. Lee, H. G. Dohlman, Coactivation of G protein signaling by cell-surface receptors and an intracellular exchange factor. Current biology : CB 18, 211-215 (2008); published online EpubFeb 12 (10.1016/j.cub.2008.01.007).
52. J. Jin, S. P. Kunapuli, Coactivation of two different G protein-coupled receptors is essential for ADP-induced platelet aggregation. Proc Natl Acad Sci USA 95, 8070-8074 (1998); published online EpubJul 7 (10.1073/pnas.95.14.8070).
53. C. Holmes, H. Smith, R. Ganderton, M. Arranz, D. Collier, J. Powell, S. Lovestone, Psychosis and aggression in Alzheimer's disease: the effect of dopamine receptor gene variation. J Neurol Neurosurg Psychiatry 71, 777-779 (2001); published online EpubDec (10.1136/jnnp.71.6.777).
54. H. Braak, K. Del Tredici, The pathological process underlying Alzheimer's disease in individuals under thirty. Acta Neuropathol 121, 171-181 (2011); published online EpubFeb (10.1007/s00401-010-0789-4).
55. T. Takeuchi, A. J. Duszkiewicz, A. Sonneborn, P. A. Spooner, M. Yamasaki, M. Watanabe, C. C. Smith, G. Fernandez, K. Deisseroth, R. W. Greene, R. G. Morris, Locus coeruleus and dopaminergic consolidation of everyday memory. Nature 537, 357-362 (2016); published online EpubSep 15 (10.1038/nature19325).
56. J. A. Crispo, Y. Fortin, D. P. Thibault, M. Emons, L. M. Bjerre, D. E. Kohen, S. Perez-Lloret, D. Mattison, A. W. Willis, D. Krewski, Trends in inpatient antiparkinson drug use in the USA, 2001-2012. Eur J Clin Pharmacol 71, 1011-1019 (2015); published online EpubAug (10.1007/s00228-015-1881-4).
57. A. K. Hornsby, Y. T. Redhead, D. J. Rees, M. S. Ratcliff, A. Reichenbach, T. Wells, L. Francis, K. Amstalden, Z. B. Andrews, J. S. Davies, Short-term calorie restriction enhances adult hippocampal neurogenesis and remote fear memory in a Ghsr-dependent manner. Psychoneuroendocrinology 63, 198-207 (2016); published online EpubJan (10.1016/j.psyneuen.2015.09.023).
58. A. Martinez-Canabal, Reconsidering hippocampal neurogenesis in Alzheimer's disease. Frontiers in neuroscience 8, 147 (2014)10.3389/fnins.2014.00147).
59. S. Forner, D. Baglietto-Vargas, A. C. Martini, L. Trujillo-Estrada, F. M. LaFerla, Synaptic Impairment in Alzheimer's Disease: A Dysregulated Symphony. Trends Neurosci 40, 347-357 (2017); published online EpubJun (10.1016/j.tins.2017.04.002).
60. J. J. Kim, D. M. Diamond, The stressed hippocampus, synaptic plasticity and lost memories. Nat Rev Neurosci 3, 453-462 (2002); published online EpubJun (10.1038/nrn849). .M. 0. Dietrich, T. L. Horvath, Hypothalamic control of energy balance: insights into the role of synaptic plasticity. Trends Neurosci 36, 65-73 (2013); published online EpubFeb (10.1016/j.tins.2012.12.005).
62. S. J. M. Baloyannis, I. Mitilineos, D. Baloyannis, I. S. Costa, V. G., The hypothalamus in Alzheimer's disease: a Golgi and electron microscope study. Am J Alzheimers Dis Other Demen 30, 478-487 (2015); published online EpubAug (10.1177/1533317514556876).
63. Y. Yin, Y. Li, W. Zhang, The growth hormone secretagogue receptor: its intracellular signaling and regulation. Int J Mol Sci 15, 4837-4855 (2014); published online EpubMar 19 (10.3390/ijms15034837).
64. W. Alzheimer's Association Calcium Hypothesis, Calcium Hypothesis of Alzheimer's disease and brain aging: A framework for integrating new evidence into a comprehensive theory of pathogenesis. Alzheimers Dement 13, 178-182 e117 (2017); published online EpubFeb (10.1016/j.jalz.2016.12.006).
65. A. B. Mathias Uhlen, Steven Can, Aled Edwards, Jan Ellenberg, Emma Lundberg, David L Rimm, Henry Rodriguez, Tara Hiltke, Michael Snyder, Tadashi Yamamoto, A proposal for validation of antibodies. Nature Methods 13, 823-827 (2016)10.1038/nmeth.3995).
66. I. Gomes, S. Sierra, L. A. Devi, Detection of Receptor Heteromerization Using In Situ Proximity Ligation Assay. Curr Protoc Pharmacol 75, 2 16 11-12 16 31 (2016); published online EpubDec 13 (10.1002/cpph.15).
67. A. B. Vincent Muczynski, Cecile Loubiére, Arnelie Harel, Ghislaine Cherel, Cecile V. Denis, Peter J. Lenting, Olivier D. Christophe, Macrophage receptor SR-AI is crucial to maintain normal plasma levels of coagulation factor X. Blood 127, 778-786 (2016)doi.org/10.1182/blood-2015-05-647032).
68. J. T. Qi Wang, Hao Chen, Heng Du, Lan Guo, Amyloid beta-mediated KIFSA deficiency disrupts anterograde axonal mitochondrial movement. Neurobiology of Disease 127, 410-418 (2019)doi.org/10.1016/j.nbd.2019.03.021).
69. V. O. N. Jean-Pierre Vilardaga, Kristina Lorenz, Sébastien Ferrandon, Zhenjie Zhuang, Martin J Lohse, Conformational cross-talk between α2A-adrenergic and μ-opioid receptors controls cell signaling. Nature Chemical Biology 4, 126-131 (2008)10.1038/nchembio.64).
70. R. G. M. Morris, Morris water maze. Scholarpedia 3, (2008) 10.4249/scholarpedia.6315).
71. M. N. Ines Lehner, Jurgen Borlak, An optimized method for the isolation and identification of membrane proteins. Electrophoresis 24, (2003)10.1002/elps.200305387).
72. W. Z. Akihiko S. Kato, Aaron D. Milstein, Mike D. Knierman, Edward R. Siuda, Joe E. Dotzlaf, Hong Yu, John E. Hale, Eric S. Nisenbaum, Roger A. Nicoll and David S. Bredt, New Transmembrane AMPA Receptor Regulatory Protein Isoform, γ-7, Differentially Regulates AMPA Receptors. The Journal of Neuroscience 27, 4969-4977 (2007)10.1523/JNEUROSCI.5561-06.2007).
73. M. N. G. Donna M Wilcock, Dave Morgan, Quantification of cerebral amyloid angiopathy and parenchymal amyloid plaques with Congo red histochemical stain. Nature Protocols 1, 1591-1595 (2006)10.1038/nprot.2006.277).

### Example 2

### Results

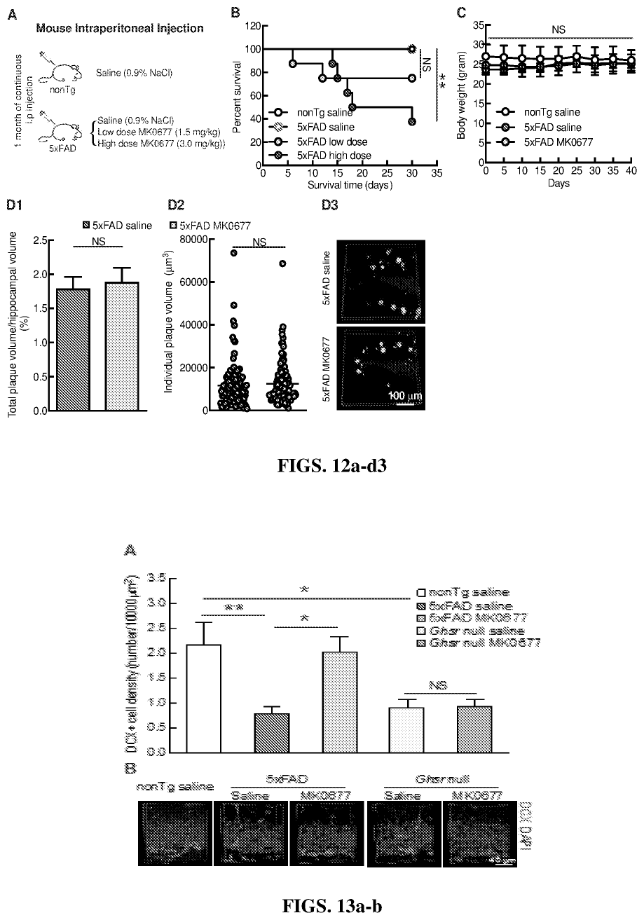

MK0677 did not mitigate hippocampal Aβ loading in 5×FAD mice. To determine whether MK0677 mitigates hippocampal lesions in 5×FAD mice, the inventor first sought to determine the optimal dose of MK0677. 5×FAD mice at 3 months old received either saline or MK0677 at 1.5 or 3 mg/kg via daily i.p. injection for 30 days (FIGS. 12a). 5×FAD mice at 3 months old have little to no hippocampal lesions and indiscernible behavioral change, both of which become prominent at 4 months of age (the endpoint of treatment) [1-5]. The doses of MK0677 were chosen based on previous studies [6-8]. Although 1.5 mg/kg MK0677 showed little effect on 5×FAD mouse survival as compared with saline treatment, MK0677 at 3 mg/kg significantly increased the mortality of 5×FAD mice (Figa. 12b). Of note, 1.5 mg/kg MK0677 did not affect mouse body weight (FIGS. 12c). The inventor therefore selected 5×FAD mice that received the 1.5 mg/kg dose of MK0677 for biological response assays. Next, he examined the impact of MK0677 treatment on hippocampal Aβ deposition in 5×FAD mice. To this end, immunofluorescent staining using the specific antibody against Aβ was performed to visualize Aβ plaques. 5×FAD mice exhibited similar levels of total Aβ plaque-occupied volume regardless of MK0677 treatment (FIGS. 12d1&d3). In addition, the measurement of the size of each single Aβ plaque showed that MK0677 administration did not significantly alter the pattern of AP accumulation in the hippocampus (FIGS. 12d2&d3). These results indicate that MK0677 at 1.5 mg/kg is not protective against hippocampal Aβ deposition, which is supported by previous studies showing minimal effects of ghrelin and its mimetic on Aβ deposition in transgenic AD mouse models [9, 10].

MK0677 promoted hippocampal neurogenesis in 5×FAD mice. In view of the neurogenic effect of GHSR1α activation [11] and defective neurogenesis in AD-relevant pathological settings [12], the inventor examined neurogenesis in the dentate gyrus by immunostaining of doublecortin (DCX), a specific marker of immature granule cells [13]. Compared with their non-TG littermates, 5×FAD mice exhibited a substantial decrease in DCX-positive cells in the dentate gyrus (FIGS. 13a&b), suggesting impaired neurogenesis in Aβ-rich milieus. 5×FAD mice with 1.5 mg/kg MK0677 treatment showed a remarkably augmented number of DCX-positive cells in their dentate gyri (FIGS. 13a&b). Importantly, the age- and gender-matched Ghsr null mice exhibited a substantially reduced number of DCX-positive cells and showed no response to MK0677′s neurogenic effect (FIGS. 13a&b), indicating the close relationship of GHSR1α signaling and neurogenesis. These findings are in agreement with previous reports that GHSR1α activation promotes hippocampal neurogenesis in AD mouse models [9].

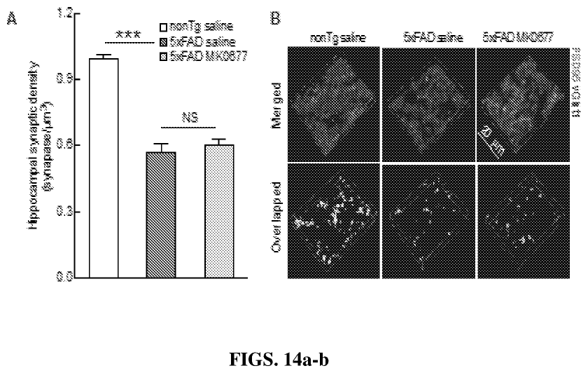

MK0677 did not alleviate hippocampal synaptic loss in 5×FAD mice. Although MK0677 at the tested dose did not affect hippocampal Aβ deposition, it was unclear whether or not elevated neurogenesis would mitigate hippocampal synaptic loss by replenishing degenerated neurons. In an effort to elucidate this, the inventor measured synaptic density in the CA1 region, which is an AD-sensitive hippocampal area [14] with pronounced synaptic loss and abundant GHSR1α expression [15-17]. Synapses were identified by colocalized staining of postsynaptic density 95 (PSD95, postsynaptic marker) and vesicular glutamate transporter 1 (vGlutl, presynaptic marker) [5]. In comparison with their non-TG counterparts, saline-treated 5×FAD mice demonstrated significantly decreased synaptic density in the CA1 region (FIGS. 14a&b). However, such 5×FAD genotypic synaptic loss was not significantly attenuated by MK0677 administration (FIGS. 14a&b).

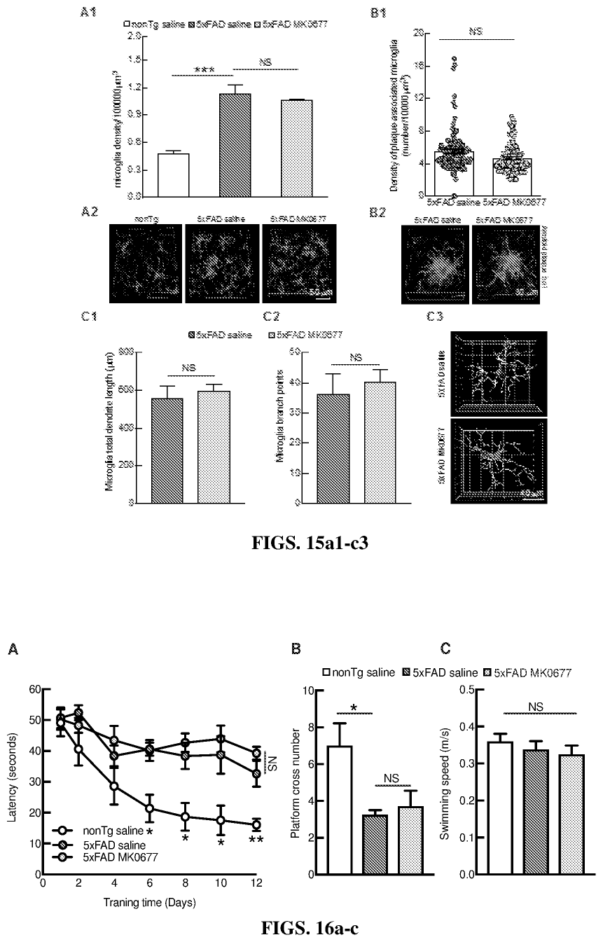

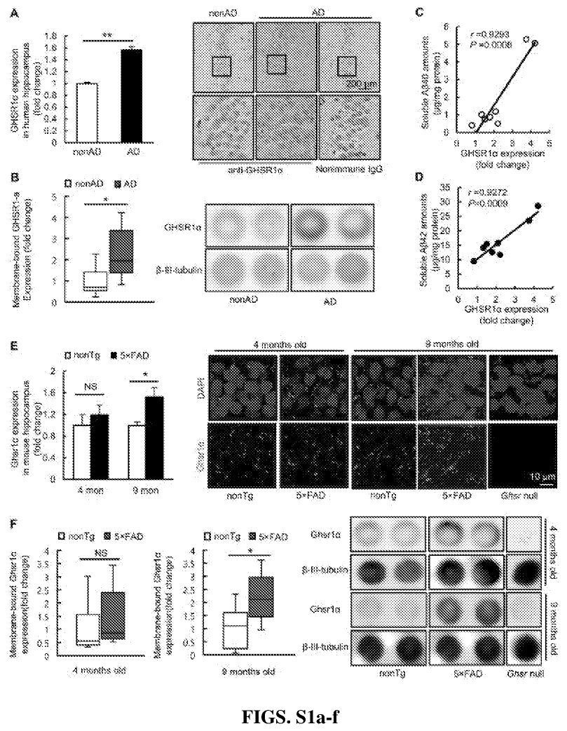

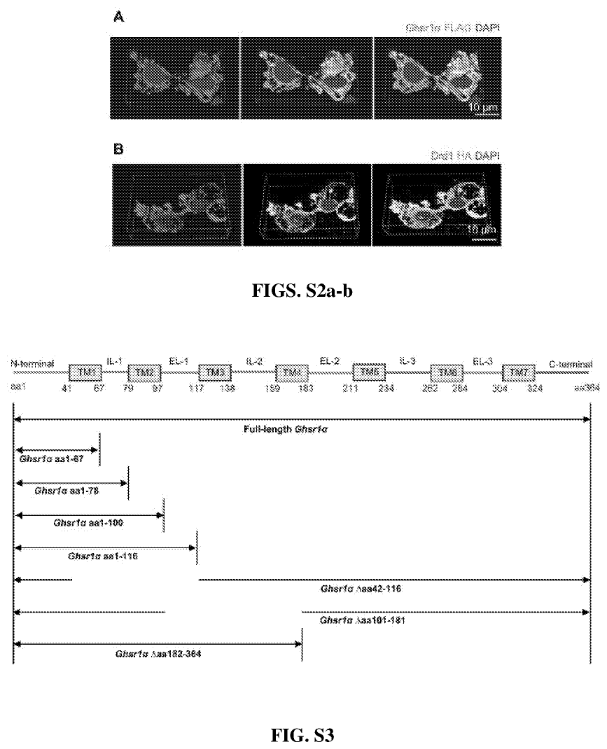

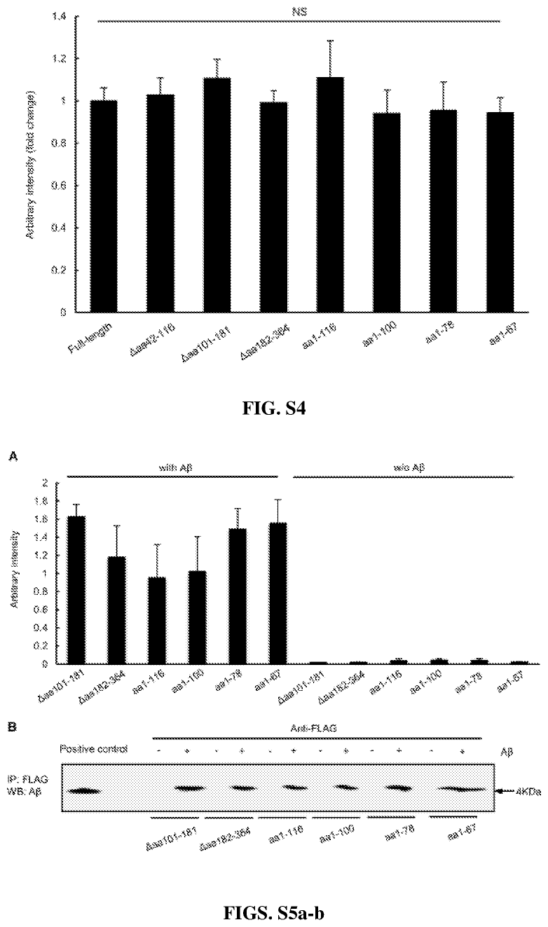

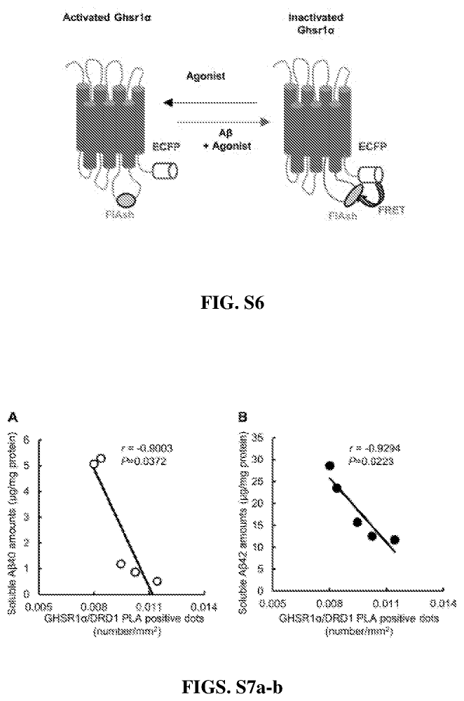

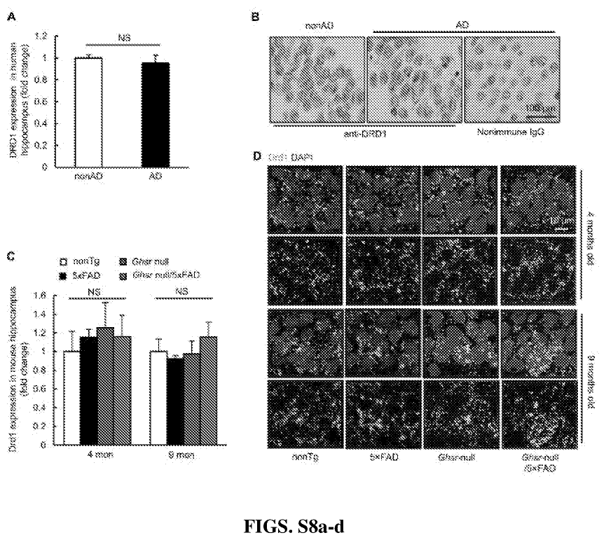

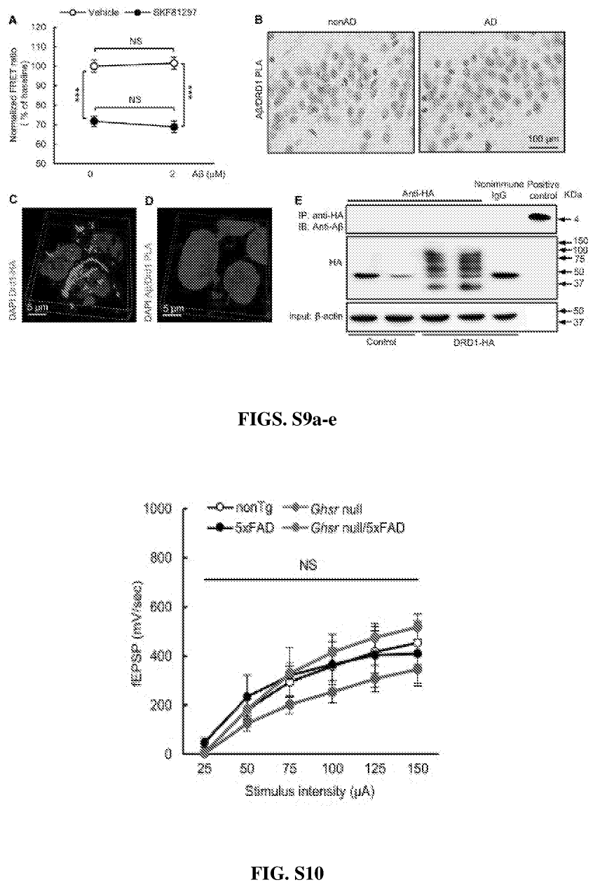

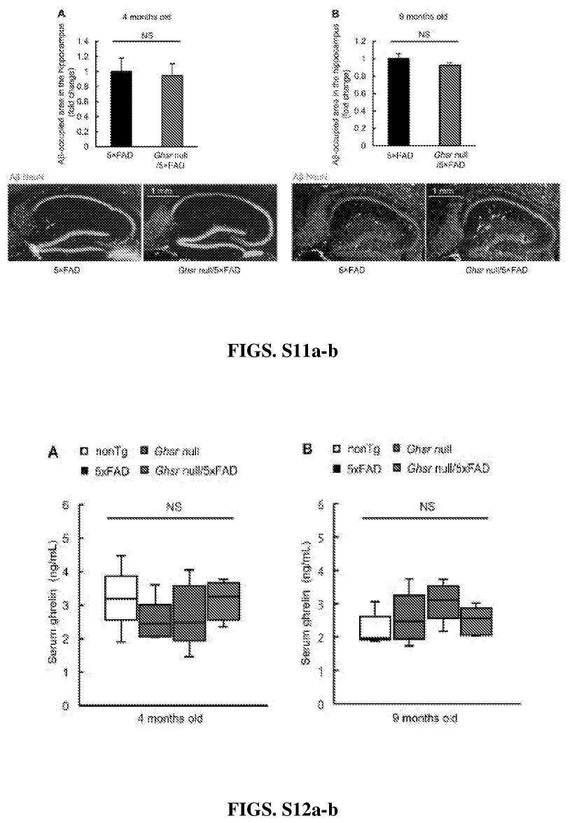

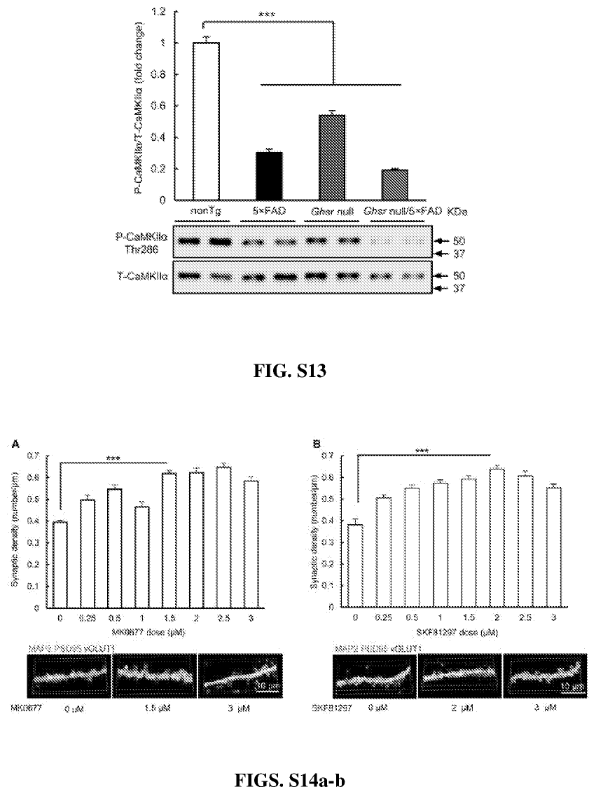

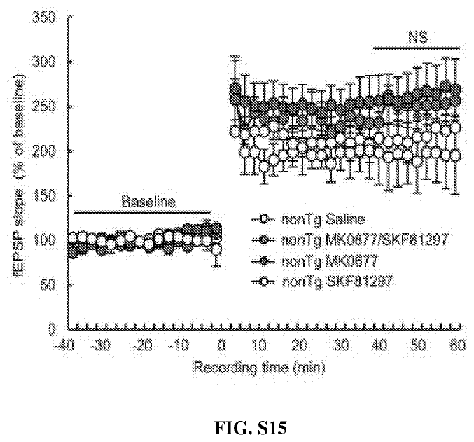

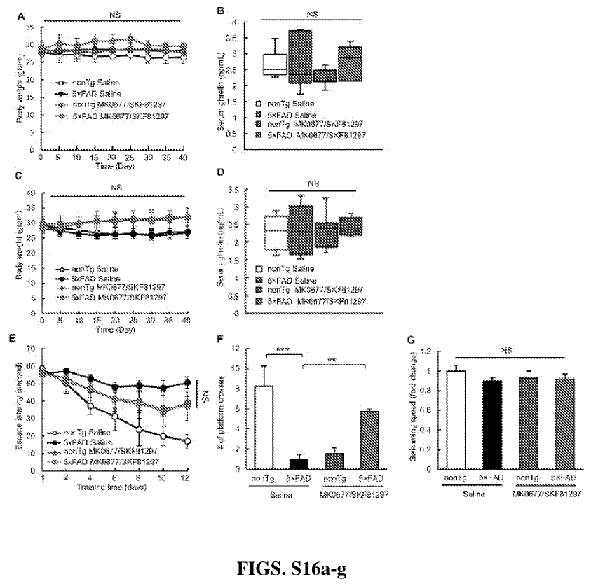

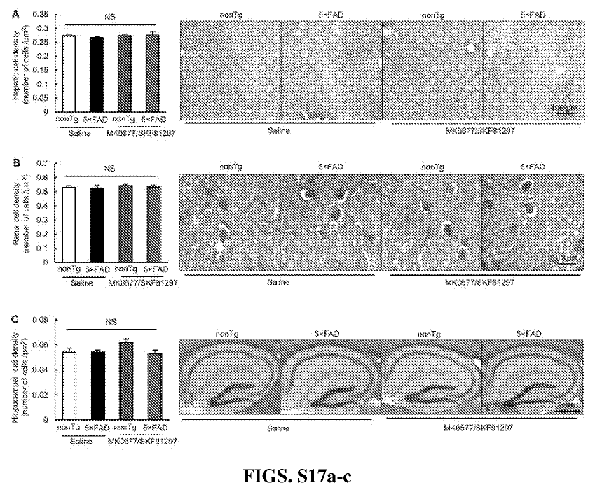

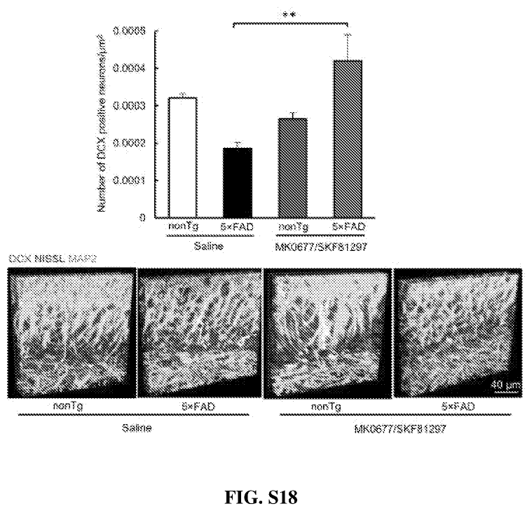

MK0677 did not attenuate microglial response to A. Microglial activation in response to Aβ toxicity and neuronal death is a pronounced AD brain pathology, which is proposed to be a key indicator of the neuroinflammation that is involved in AD pathogenesis [18-20]. To determine whether MK0677 affects hippocampal microglial activation, the inventor stained microglia using an antibody against a microglial marker, ionized calcium-binding adaptor molecule 1 (Iba-1) [21]. As opposed to their non-TG littermates, saline-treated 5×FAD mice demonstrated substantially increased microglial density in the hippocampus (FIGS. 15a1&a). Such genotypic change was not prevented by MK0677 treatment (FIGS. 15a1&a2). Moreover, saline- and MK0677-treated 5×FAD mice showed no significant difference in the density of plaque-associated microglia (FIGS. 15b1&b2), length of total process (FIGS. 15c1&c3), or the number of branch points (FIGS. 15c2&c3). These results do not demonstrate any effect of MK0677 on hippocampal microglial response to AP.

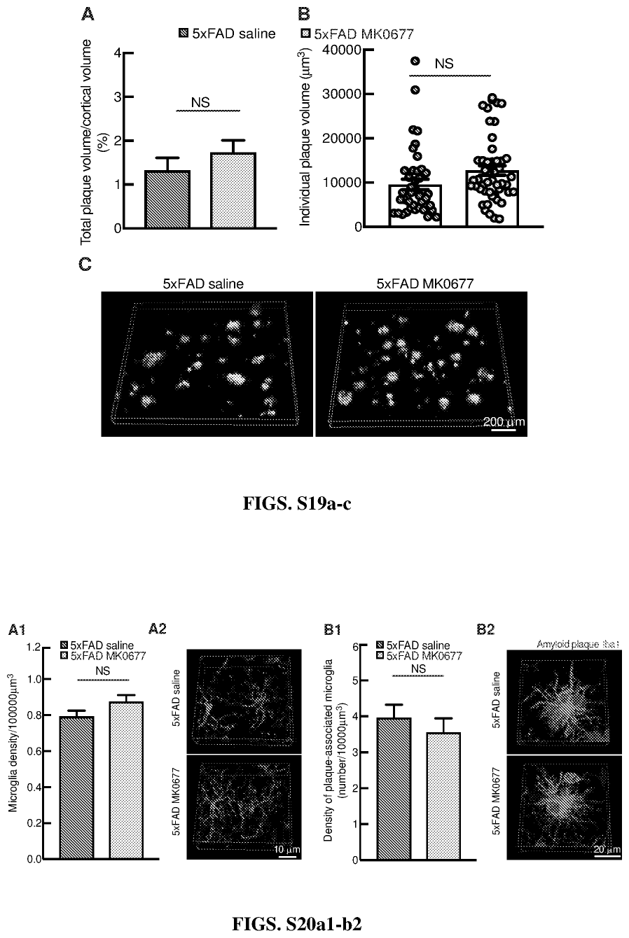

MK0677 did not improve spatial learning and memory in 5×FAD mice. To determine whether MK0677 preserves 5×FAD mouse cognition, especially AD-sensitive hippocampus-associated cognition, the inventor examined mouse spatial reference learning and memory using the Morris water maze. Saline-treated 5×FAD mice exhibited pronounced damage to spatial reference learning (FIG. 16a) and memory (FIG. 16b), which were in sharp contrast with their non-TG littermates (FIGS. 16a&b). MK0677 treatment failed to show any protective effect with regards to genotypic cognitive impairment in 5×FAD mice (FIGS. 16a&b). Of note, the administration of MK0677 did not affect the swimming speed of the experimental mice (FIG. 16c). Such findings echo the ineffectual function of MK0677 treatment on the development of hippocampal synaptic density in 5×FAD mice.

### Discussion

Although the detailed mechanisms underlying the ghrelin/GHSR1α system's regulation of hippocampal synaptic physiology remain to be elucidated, three pathways have been proposed including direct modulation, metabolic regulation, and neurogenic effect. The direct modulation of hippocampal synaptic function through GHSR1α is evidenced by the observation that GHSR1α activation promotes α-amino-3-hydroxy-5-methyl-4-isoxazolepropionic acid receptor (AMPAR) trafficking, resulting in enhanced AMPAR incorporation into hippocampal synapses [22]. Such an effect is associated with the activation of protein kinase A (PKA), which is a key in GHSR1α downstream signaling [22, 23]. Moreover, Kern and colleagues have shown that GHSR1α heteromerizes with hippocampal dopamine receptor D1 (DRD1), instigating DRD1 Gαq-Ca²⁺ signaling, and culminating in the initiation of hippocampal synaptic plasticity [24]. In addition to direct action, the ghrelin/GHSR1α system may modulate hippocampal function through its regulation of brain metabolism [25]. Indeed, the pituitary, the hypothalamus and the hippocampus are GHSR1α -expressing regions in central nervous system (CNS) [15, 26]. GHSR1α in the pituitary and the hypothalamic arcuate nucleus as well as ventromedial nuclei (VMN) regulates the production and secretion of multiple critical hormones such as growth hormone (GH), adrenocorticotropin (ACTH), and thyroid-stimulating hormone (TSH), as well as others that affect food intake, metabolism, and stress responses [27-30]. These metabolic effects of the ghrelin/GHSR1α system may promote hippocampal synaptic function and facilitate hippocampus-associated cognition [31, 32]. Lastly, recent studies have also highlighted the link between GHSR1α activation and enhanced hippocampal neurogenesis and its potential contribution to hippocampal synaptic regulation in health and disease [33]. Since AMPAR deregulation [34-36], brain dysmetabolism [37, 38], and neurogenesis defects [12, 39, 40] are featured pathological changes accompanying AD, GHSR1α activation has its potential to remedy hippocampal synaptic failure in AD.

In previous studies, different groups have validated the beneficial effects of GHSR1α ligands, including ghrelin and acyl ghrelin, as well as GHSR1α agonists, including MK0677 and LY444711, on hippocampal neurogenesis, long-term potentiation, and cognition against AP toxicity in rodent models [9, 10, 41, 42]. Although the inventor has observed impaired neurogenesis in the dentate gyms in 5×FAD mice and the neurogenic effect of MK0677 treatment, which is in agreement with a previous study [9], these results do not support the protective effect of GHSR1α activation against the development of Aβ-induced synaptic deficits or cognitive impairment. The ineffectiveness of MK0677 determined in the inventor's study is in line with a previous clinical trial using MK0677 for the treatment of AD [7]. Such discrepancies between previous studies and ours may arise from the use of different types of experimental systems. Two of the aforementioned studies employed Aβ40- or 42-injected mouse or rat models [41, 42]. The acute Aβ toxicity model may not represent the complexity of neuronal changes in response to chronic Aβ production and accumulation. Another group challenged a transgenic AD mouse model on a high glycemic index (GI) diet [10]; therefore, the protective effect of GHSR1α activation determined in this study may, at least in part, result from the influence of a GHSR1α agonist against high glucose-induced stress in Aβ-rich settings. It has also been proposed that hippocampal neurogenesis could be a potential avenue for AD treatment[43, 44]. Despite this theory, the enhanced hippocampal neurogenesis by the administration of MK0677 failed to alleviate synaptic loss in the hippocampal CA1 region or mouse cognitive impairment in this study. It cannot be excluded that MK0677-enhanced neurogenesis is not potent enough to replenish a sufficient number of neurons capable of attenuating hippocampal lesions in 5×FAD mice. In fact, whether or not neurogenesis can rescue hippocampal function in diseases has yet to reach a consensus. It also remains to be seen whether (and if so, how many) hippocampal progenitor cells can differentiate into granule neurons and subsequently form functional synaptic neural networks [44, 45]. Moreover, it is unclear whether MK0677-promoted neurogenesis implements any yet-undetermined influence(s) on brain function, especially in 5×FAD mice. Further studies on this issue could serve to bring light to these questions.

Lastly, Jeong and colleagues showed that MK0677 decreases Aβ deposition and microglial activation in 5×FAD mouse neocortices [6]. In current study, the inventor did not detect any effect of MK0677 on hippocampal Aβ loading or microglial activation. In fact, the inventor's quantitative analysis failed to show any influence of MK0677 on total plaque-occupied volume (FIGS. S19a&c), single plaque size (FIGS. S19b&c), the density of total microglia (FIGS. 20a1&a2), or the density of plaque-associated microglia (FIGS. 20b1&b2) in the neocortex of MK0677-treated 5×FAD mice. The inventor's observations of the inconsequential impact of ghrelin or other GHSR1α agonist on brain Aβ loading is in agreement with the results from several previous studies [9, 10]. The inventor cannot exclude the possibility that MK0677 at high doses, such as 5 mg/kg used in Jeong's study [6], would affect Aβ production and/or deposition. However, he found that MK0677 at 3 mg/kg significantly increased mortality of 5×FAD mice, which prevented the use of higher doses of MK0677. Therefore, it is suggested to err on the side of caution when using high doses of MK0677 as a therapeutic strategy for AD treatment even if it is effective in the reduction of brain amyloidosis.

In summary, the inventor has shown that GHSR1α activation by its agonist MK0677 promotes hippocampal neurogenesis but fails to mitigate hippocampal synaptic lesions and cognitive impairment in 5×FAD mice. Thse findings correlate with those from a failed clinical trial [7] regardless of the argument that said trial treated the patients at a critically advanced stage. To counter this, the inventor administrated MK0677 to mice at the asymptomatic stage when there is little to mild brain Aβ deposition and indiscernible synaptic injury and failed to detect the preventive effect of GHSR1α activation. Therefore, GHSR1α activation through its agonists does not seem to be an effective strategy for AD prevention. Of note, in a recent study the inventor determined increased serum levels of acyl ghrelin in patients with mild cognitive impairment (MCI) due to AD, which negatively correlates with the patients' cognitive performance [46]. This observation, together with the ineffectiveness of MK0677 as seen in this study and a previous clinical trial [7], seems to suggest a suppressed GHSR1α response to its ligand- or agonist-induced activation in AD-relevant pathophysiological settings. In this case, a comprehensive study on GHSR1α functional status, especially GHSR1α response to its activating factors in AD-related conditions will help to address this issue. Nevertheless, the simplest interpretation of these results is that GHSR1α activation by MK0677 alone has limited therapeutic potential for AD prevention and treatment.

### Materials and Methods

Mice and agonist treatment. 5×FAD mice (B6SJL-Tg (APPSwFlLon, PSEN1*M146L*L286V) 6799Vas/Mmjax) were obtained from Jackson Laboratory and crossed with B6SJL-Tg mice [1]. Ghsr null mice were originally from Dr. Jeffrey M. Zigman at UT Southwestern Medical Center and bred for experimental use [47]. Mouse breeding and usage was performed in accordance with the guidelines of the University of Texas at Dallas (UTD) Institutional Animal Care and Use Committee (IACUC) and National Institutes of Health (NIH). Mice were used in pairs of age-matched non-transgenic mice (non-TG). Genotypes of animals were confirmed using PCR and/or amyloid plaque staining. The number of mice used was determined by previous data and calculations to ensure that the minimal number of mice as required was used in these experiments. 3-month-old non-TG mice received daily intraperitoneal (i.p.) injections of saline (sterilized 0.9% NaCl) for one month. The age- and gender-matched 5×FAD mice and the littermates non-TG mice received daily i.p. injections of either saline (sterilized 0.9% NaCl) or MK0677 (Tocris, #5272, at doses of 1.5 or 3 mg/kg) diluted in saline for one month. Ghsr null mice received daily i.p. injections of saline (sterilized 0.9% NaCl) or MK0677 (Tocris, #5272, 1.5 mg/kg) diluted in saline for one month. Mouse body weight was recorded every five days until euthanasia. After the 1-month treatment, the mice were subjected to the Morris water maze behavioral test and then euthanized for tissue collection following IACUC-approved procedures.

Immunocytochemistry. Frozen sections were prepared as previously described [48]. Mouse brains were dissected and immediately fixed in 4% paraformaldehyde (PFA) (Sigma-Aldrich) for 24-26 hours at 4° C. followed by keeping in 30% sucrose for 24-26 hours at 4° C. The slices were blocked with blocking buffer (5% goat or donkey serum as needed, 0.3% Triton X-100 in PBS, pH 7.4) for 1 hour, then incubated with primary antibodies at room temperature overnight. The following antibodies were used: rabbit-anti-β-amyloid (CST, #8243, 1:1,000) for Aβ deposition detection, mouse-anti-Doublecortin (Santa Cruz Biotechnology, #sc271390, 1:100), rabbit-anti-PSD95 (CST, #3450, 1:400), guinea pig-anti-vGlutl (Synaptic system, #135304, 1:400), goat-anti-Ibal (abcam, #ab5076, 1:600). After washing with PBS, the slices were probed with appropriate cross-adsorbed secondary antibodies conjugated to Alexa Fluor 488 and/or Alexa Fluor 594 (Thermo Fisher Scientific, 1:400). Nuclei were labeled with 4′,6-diamidino-2-phenylindole (DAPI, Thermo Fisher Scientific, #R37606, 1 drop/500 ul) as needed. Images were collected on a Nikon confocal microscope.

Amyloid plaque deposition analysis. For amyloid plaque deposition analysis, amyloid plaque channel was subjected to “Binary” then “Define Threshold” in NIS element software. The threshold was set to include all the positive staining and the same setting was applied to all images. Volume measurement was then performed for all the plaques as well as each single plaque in both the hippocampal and cortical areas using the ROI selection option. Amyloid plaque-occupied percentage in the hippocampus or cortex and the average individual plaque volume was used to compare amyloid plaque deposition between 5×FAD saline and MK0677 treatment groups.

Neurogenesis analysis. Neurogenesis in the hippocampal dentate gyms (DG) area was presented by performing doublecortin (DCX) staining in fixed brain slices as previously described [9]. DCX-positive (DCX⁺) cell number was counted manually. The DG area was measured using NIS element software. DCX cell density was calculated by dividing the DCX cell number by the DG area to evaluate neurogenesis.

Synaptic density analysis. For synaptic density analysis, vesicular glutamate transporter 1(vGlutl)- and postsynaptic density 95 (PSD95)-stained channels were saved as two binary layers, the threshold for each channel defined separately and then applied to all the images. The overlapping areas were analyzed by using the “AND” operation in the “Binary Operation” dialogs of NIS element software [5]. Hippocampal volume was measured as described in the previous section (amyloid plaque deposition analysis). The overlapping dots represent synapses and average synapse number per selected area volume was calculated to represent synaptic density.

Microglia morphology and plaque-associated microglia density analysis. Analysis of microglia morphology was done by producing three-dimensional (3D) reconstructions of cells using Imaris software (Bitplane, Imaris×64 9.0.2)[49]. Using the Filament algorithm, the software was able to calculate the length of each cell dendrite and number of branch points. This was done by first selecting the appropriate region of interest for the desired cell. If there were nearby cells, the somas of those cells were included in the region of interest. Since the software builds the dendrites based on extension from the largest set diameter, doing this can prevent unwanted filaments bridging the dendrites of two or more cells. The dendrite diameter parameters were set with the largest diameter being the soma size and the smallest diameter at 0.5 μm. To further prevent unwanted connections, the option to ‘Remove disconnected segments’ was used in the dendrite building menu with the distance threshold set at 5 μm. After the algorithm finished detecting the dendrites, non-specifically detected filaments were manually trimmed. Dendrite length and branch points were then calculated by the software. Plaque-associated microglia density was analyzed by using NIS element software. Microglia were counted under the following rule: if more than 50% of the microglia soma was within a plaque, it was counted as one plaque-associated microglia. Plaque volume was analyzed as described in the previous section (amyloid plaque deposition analysis).

Mouse behavioral test. The Morris water maze test was performed as previously described in order to test changes in mouse spatial learning and memory [3, 50]. Mice were randomly grouped by gender, genotypes and treatments (which the experimenter was blind to) and then transferred to the testing environment at least 0.5 hours before tests. During the test, mice were trained to find a hidden platform (20 cm diameter) in an open swimming pool (200 cm diameter) filled with 21° C. water (non-toxic white dye was used to hide the platform). Four trials were performed each day for 12 days. Each trial started at a different position (NW, N, E, SE) while the platform was kept in the same location (SW). Each trial lasted 60 seconds followed by 30 seconds during which the mice were left on the platform to memorize its location. After 12 days of training, mice were subjected to a 60-second probe test, which started from a new position (NE) and the platform was removed. The frequency with which mice swam across the previous platform location as well as swimming speed and latency was recorded and analyzed with HVS Image software (HVS Image 2015). Probe results were analyzed using the platform location crossed frequency value.

Statistics. Statistical analyses were performed using GraphPad Software (Prism 7). For mouse lifespan, body weight, neurogenesis, synaptic density, hippocampal microglia density, Morris water maze learning curve, probe, and swimming speed, two-way ANOVA followed by Bonferroni post hoc analysis were performed. For hippocampal and cortical total plaque-occupied percentage, individual plaque volume, plaque-associated microglia density, microglia dendrite length, and microglia branch point number, unpaired Student's t-test were performed. Results are presented as mean ±s.e.m. T-value and p-value were used to represent the variation and the significance level, respectively. P<0.05 was considered as statistically significant. * P<0.05, ** P<0.01, *** P<0.001. NS: not significant.

### References for Example 2

The following references, to the extent that they provide exemplary procedural or other details supplementary to those set forth herein, are specifically incorporated herein by reference.
[1] W. A. Eimer and R. Vassar, “Neuron loss in the 5XFAD mouse model of Alzheimer's disease correlates with intraneuronal Abeta42 accumulation and Caspase-3 activation,” Mol Neurodegener, vol. 8, p. 2, Jan. 14 2013, doi: 10.1186/1750-1326-8-2.
[2] L. Wang et al., “Synaptosomal Mitochondrial Dysfunction in 5×FAD Mouse Model of Alzheimer's Disease,” PLoS One, vol. 11, no. 3, p. e0150441, 2016, doi: 10.1371/journal.pone.0150441.
[3] L. Lu et al., “Transient Cerebral Ischemia Promotes Brain Mitochondrial Dysfunction and Exacerbates Cognitive Impairments in Young 5×FAD Mice,” PLoS One, vol. 10, no. 12, p. e0144068, 2015, doi: 10.1371/journal.pone.0144068.
[4] E. Gauba, H. Chen, L. Guo, and H. Du, “Cyclophilin D deficiency attenuates mitochondrial F1Fo ATP synthase dysfunction via OSCP in Alzheimer's disease,” Neurobiol Dis, vol. 121, pp. 138-147, January 2019, doi: 10.1016/j.nbd.2018.09.020.
[5] S. J. Beck et al., “Deregulation of mitochondrial F1FO-ATP synthase via OSCP in Alzheimer's disease,” Nat Commun, vol. 7, p. 11483, May 6 2016, doi: 10.1038/ncomms 11483.
[6] Y. O. Jeong et al., “MK-0677, a Ghrelin Agonist, Alleviates Amyloid Beta-Related Pathology in 5XFAD Mice, an Animal Model of Alzheimer's Disease,” Int J Mol Sci, vol. 19, no. 6, Jun 18 2018, doi: 10.3390/ijms19061800.
[7] J. J. Sevigny et al., “Growth hormone secretagogue MK-677: no clinical effect on AD progression in a randomized trial,” Neurology, vol. 71, no. 21, pp. 1702-8, Nov. 18, 2008, doi: 10.1212/01.wn1.0000335163.88054.e7.
[8] H. Zheng et al., “Somatostatin receptor subtype 2 knockout mice are refractory to growth hormone-negative feedback on arcuate neurons,” Mol Endocrinol, vol. 11, no. 11, pp. 1709-17, October 1997, doi: 10.1210/mend.11.11.0016.
[9] M. Moon, M. Y. Cha, and I. Mook-Jung, “Impaired hippocampal neurogenesis and its enhancement with ghrelin in 5×FAD mice,” J Alzheimers Dis, vol. 41, no. 1, pp. 233-41, 2014, doi: 10.3233/JAD-132417.
[10] N. Kunath, T. van Groen, D. B. Allison, A. Kumar, M. Dozier-Sharpe, and I. Kadish, “Ghrelin agonist does not foster insulin resistance but improves cognition in an Alzheimer's disease mouse model,” Scientific reports, vol. 5, p. 11452, Jun 19 2015, doi: 10.1038/srep11452.
[11] C. Kim, S. Kim, and S. Park, “Neurogenic Effects of Ghrelin on the Hippocampus,” Int J Mol Sci, vol. 18, no. 3, Mar. 8, 2017, doi: 10.3390/ijms18030588.
[12] E. P. Moreno-Jimenez et al., “Adult hippocampal neurogenesis is abundant in neurologically healthy subjects and drops sharply in patients with Alzheimer's disease,” Nat Med, vol. 25, no. 4, pp. 554-560, April 2019, doi: 10.1038/s41591-019-0375-9.
[13] S. Couillard-Despres et al., “Doublecortin expression levels in adult brain reflect neurogenesis,” Eur J Neurosci, vol. 21, no. 1, pp. 1-14, January 2005, doi: 10.1111/j.1460-9568.2004.03813.x.
[14] M. Padurariu, A. Ciobica, I. Mavroudis, D. Fotiou, and S. Baloyannis, “Hippocampal neuronal loss in the CA1 and CA3 areas of Alzheimer's disease patients,” Psychiatr Danub, vol. 24, no. 2, pp. 152-8, June 2012.
[15] B. K. W. Mani, A. K. Lopez Soto, E. J. Raingo, J. Lee, C. E. Perello, M. Andrews, Z. B. Zigman, J. M., “Neuroanatomical characterization of a growth hormone secretagogue receptor-green fluorescent protein reporter mouse,” J Comp Neurol, vol. 522, no. 16, pp. 3644-66, Nov 1 2014, doi: 10.1002/cne.23627.
[16] T. M. H. Hsu, J. D. Konanur, V. R. Noble, E. E. Suarez, A. N. Thai, J. Nakamoto, E. M. Kanoski, S. E., “Hippocampus ghrelin signaling mediates appetite through lateral hypothalamic orexin pathways,” eLife, vol. 4, Dec. 15, 2015, doi: 10.7554/eLife.11190.
[17] B. K. Mani et al., “The role of ghrelin-responsive mediobasal hypothalamic neurons in mediating feeding responses to fasting,” Mol Metab, vol. 6, no. 8, pp. 882-896, August 2017, doi: 10.1016/j.molmet.2017.06.011.
[18] H. Keren-Shaul et al., “A Unique Microglia Type Associated with Restricting Development of Alzheimer's Disease,” Cell, vol. 169, no. 7, pp. 1276-1290 e17, Jun. 15 2017, doi: 10.1016/j.cell.2017.05.018.
[19] P. D. Wes, F. A. Sayed, F. Bard, and L. Gan, “Targeting microglia for the treatment of Alzheimer's Disease,” Glia, vol. 64, no. 10, pp. 1710-32, October 2016, doi: 10.1002/glia.22988.
[20] I. Visan, “Alzheimer's disease microglia,” Nat Immunol, vol. 18, no. 8, p. 876, Jul. 19 2017, doi: 10.1038/ni.3802.
[21] D. Ito, Y. Imai, K. Ohsawa, K. Nakajima, Y. Fukuuchi, and S. Kohsaka, “Microglia-specific localisation of a novel calcium binding protein, Ibal,” Brain Res Mol Brain Res, vol. 57, no. 1, pp. 1-9, Junl 1 1998.
[22] L. F. Ribeiro et al., “Ghrelin triggers the synaptic incorporation of AMPA receptors in the hippocampus,” Proc Natl Acad Sci USA, vol. 111, no. 1, pp. E149-58, Jan. 7, 2014, doi: 10.1073/pnas.1313798111.
[23] J. N. Cuellar and M. Isokawa, “Ghrelin-induced activation of cAMP signal transduction and its negative regulation by endocannabinoids in the hippocampus,” Neuropharmacology, vol. 60, no. 6, pp. 842-51, May 2011, doi: 10.1016/j .neuropharm.2010.12.024.
[24] A. Kern, M. Mavrikaki, C. Ullrich, R. Albarran-Zeckler, A. F. Brantley, and R. G. Smith, “Hippocampal Dopamine/DRD1 Signaling Dependent on the Ghrelin Receptor,” Cell, vol. 163, no. 5, pp. 1176-1190, Nov. 19, 2015, doi: 10.1016/j.cell.2015.10.062.
[25] S. Diano et al., “Ghrelin controls hippocampal spine synapse density and memory performance,” Nat Neurosci, vol. 9, no. 3, pp. 381-8, March 2006, doi: 10.1038/nn1656.
[26] X. M. Guan et al., “Distribution of mRNA encoding the growth hormone secretagogue receptor in brain and peripheral tissues,” Brain Res Mol Brain Res, vol. 48, no. 1, pp. 23-9, August 1997.
[27] C. L. Grey, L. Grayfer, M. Belosevic, and J. P. Chang, “Ghrelin stimulation of gonadotropin (LH) release from goldfish pituitary cells: presence of the growth hormone secretagogue receptor (GHS-R1a) and involvement of voltage-sensitive Ca2+ channels,” Mol Cell Endocrinol, vol. 317, no. 1-2, pp. 64-77, Apr. 12, 2010, doi: 10.1016/j.mce.2009.12.024.
[28] M. Kojima, H. Hosoda, Y. Date, M. Nakazato, H. Matsuo, and K. Kangawa, “Ghrelin is a growth-hormone-releasing acylated peptide from stomach,” Nature, vol. 402, no. 6762, pp. 656-60, Dec. 9, 1999, doi: 10.1038/45230.
[29] L. Gianotti et al., “Recombinant human IGF-I does not modify the ACTH and cortisol responses to hCRH and hexarelin, a peptidyl GH secretagogue, in humans,” J Endocrinol Invest, vol. 24, no. 2, pp. 67-71, February 2001, doi: 10.1007/BF03343815.
[30] K. Takaya et al., “Ghrelin strongly stimulates growth hormone release in humans,” J Clin Endocrinol Metab, vol. 85, no. 12, pp. 4908-11, Dec 2000, doi: 10.1210/jcem.85.12.7167.
[31] G. McGregor and J. Harvey, “Regulation of Hippocampal Synaptic Function by the Metabolic Hormone, Leptin: Implications for Health and Neurodegenerative Disease,” Front Cell Neurosci, vol. 12, p. 340, 2018, doi: 10.3389/fnce1.2018.00340.
[32] G. McGregor, Y. Malekizadeh, and J. Harvey, “Minireview: Food for thought: regulation of synaptic function by metabolic hormones,” Mol Endocrinol, vol. 29, no. 1, pp. 3-13, January 2015, doi: 10.1210/me.2014-1328.
[33] H. J. Huang et al., “The protective effects of Ghrelin/GHSR on hippocampal neurogenesis in CUMS mice,” Neuropharmacology, vol. 155, pp. 31-43, Sep. 1, 2019, doi: 10.1016/j.neuropharm.2019.05.013.
[34] S. Jurado, “AMPA Receptor Trafficking in Natural and Pathological Aging,” Front Mol Neurosci, vol. 10, p. 446, 2017, doi: 10.3389/fnmo1.2017.00446.
[35] Y. Zhang, 0. Guo, Y. Huo, G. Wang, and H. Y. Man, “Amyloid-beta Induces AMPA Receptor Ubiquitination and Degradation in Primary Neurons and Human Brains of Alzheimer's Disease,” J Alzheimers Dis, vol. 62, no. 4, pp. 1789-1801, 2018, doi: 10.3233/JAD-170879.
[36] S. Guntupalli, J. Widagdo, and V. Anggono, “Amyloid-beta-Induced Dysregulation of AMPA Receptor Trafficking,” Neural Plast, vol. 2016, p. 3204519, 2016, doi: 10.1155/2016/3204519.
[37] S. C. Thomas, A. Alhasawi, V. P. Appanna, C. Auger, and V. D. Appanna, “Brain metabolism and Alzheimer's disease: the prospect of a metabolite-based therapy,” J Nutr Health Aging, vol. 19, no. 1, pp. 58-63, January 2015, doi: 10.1007/s12603-014-0511-7.
[38] S. M. de la Monte and M. Tong, “Brain metabolic dysfunction at the core of Alzheimer's disease,” Biochem Pharmacol, vol. 88, no. 4, pp. 548-59, Apr. 15, 2014, doi: 10.1016/j.bcp.2013.12.012.
[39] A. Maruszak, A. Pilarski, T. Murphy, N. Branch, and S. Thuret, “Hippocampal neurogenesis in Alzheimer's disease: is there a role for dietary modulation?,” J Alzheimers Dis, vol. 38, no. 1, pp. 11-38, 2014, doi: 10.3233/JAD-131004.
[40] Y. Mu and F. H. Gage, “Adult hippocampal neurogenesis and its role in Alzheimer's disease,” Mol Neurodegener, vol. 6, p. 85, Dec 22 2011, doi: 10.1186/1750-1326-6-85.
[41] M. Eslami, B. Sadeghi, and F. Goshadrou, “Chronic ghrelin administration restores hippocampal long-term potentiation and ameliorates memory impairment in rat model of Alzheimer's disease,” Hippocampus, vol. 28, no. 10, pp. 724-734, Oct 2018, doi: 10.1002/hipo.23002.
[42] V. V. Santos et al., “Acyl ghrelin improves cognition, synaptic plasticity deficits and neuroinflammation following amyloid beta (Abetal-40) administration in mice,” J Neuroendocrinol, vol. 29, no. 5, May 2017, doi: 10.1111/jne.12476.
[43] K. A. Sailor, G. L. Ming, and H. Song, “Neurogenesis as a potential therapeutic strategy for neurodegenerative diseases,” Expert Opin Biol Ther, vol. 6, no. 9, pp. 879-90, September 2006, doi: 10.1517/14712598.6.9.879.
[44] N. A. DeCarolis and A. J. Eisch, “Hippocampal neurogenesis as a target for the treatment of mental illness: a critical evaluation,” Neuropharmacology, vol. 58, no. 6, pp. 884-93, May 2010, doi: 10.1016/j.neuropharm.2009.12.013.
[45] P. Baptista and J. P. Andrade, “Adult Hippocampal Neurogenesis: Regulation and Possible Functional and Clinical Correlates,” Front Neuroanat, vol. 12, p. 44, 2018, doi: 10.3389/fnana.2018.00044.
[46] X. Cao, M. Zhu, Y. He, W. Chu, Y. Du, and H. Du, “Increased Serum Acylated Ghrelin Levels in Patients with Mild Cognitive Impairment,” J Alzheimers Dis, vol. 61, no. 2, pp. 545-552, 2018, doi: 10.3233/JAD-170721.
[47] Y. N. Jeffrey M. Zigman, Roberto Coppari, Nina Balthasar, Jacob N. Marcus, Charlotte E. Lee, Juli E. Jones, Amy E. Deysher, Amanda R. Waxman, Ryan D. White, Todd D. Williams, Jennifer L. Lachey, Randy J. Seeley, Bradford B. Lowell, Joel K. Elmquist, “Mice lacking ghrelin receptors resist the development of diet-induced obesity,” The Journal of Clinical Investigation, vol. 115, pp. 3564-3572, 2005.
[48] J. T. Qi Wang, Hao Chen, Heng Du, Lan Guo, “Amyloid beta-mediated KIFSA deficiency disrupts anterograde axonal mitochondrial movement,” Neurobiology of Disease, vol. 127, pp. 410-418, 2019, doi: doi.org/10.1016/j.nbd.2019.03.021.
[49] D. PelinCengiz, Jayadevi H.Chandrashekhar, VishalChanan, JacobBogost, AlexWaldmanb, Beccallovak, Douglas B.Kintner, Peter A.Ferrazzano, “Developmental differences in microglia morphology and gene expression during normal brain development and in response to hypoxia-ischemic,” Neurochemistry International, vol. 127, pp. 137-147, 2018, doi: 10.1016/j.neuint.2018.12.016.
[50] R. G. M. Morris, “Morris water maze,” Scholarpedia, vol. 3, 2008, doi: 10.4249/scholarpedia.6315.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed:

1. A method for treatment of hippocampal synaptic dysfunction and Alzheimer's disease in a patient comprising the step of administering one or more drug compositions to the patient wherein the one or more drug compositions are configured to promote the heteromerization of GHSR1αand DRD1.

2. The method of claim 1, wherein the method of administering the one or more drug compositions includes the step of simultaneously administering an agonist of GHSR1αand an agonist of DRD1 to the patient.

3. The method of claim 2, further comprising the step of selecting the agonist of GHSR1αfrom the group consisting of: Adenosine, Alexamorelin, Anamorelin, Capromorelin, CP-464709, Cortistatin-14, Examorelin (hexarelin), Ghrelin (lenomorelin), GHRP-1, GHRP-3, GHRP-4, GHRP-5, GHRP-6, Ibutamoren (MK-0677), Ipamorelin, L-692,585, LY-426410, LY-444711, Macimorelin, Pralmorelin, (GHRP-2), Relamorelin, SM-130,686, Tabimorelin and Ulimorelin.

4. The method of claim 2, further comprising the step of selecting the agonist of DRD1 from the group consisting of: dopamine, A-86929, Dihydrexidine, Dinapsoline, Dinoxyline, Doxanthrine, SKF-81297, SKF-82958, SKF-38393, Fenoldopam, 6-Br-APB, Stepholidine, A-68930, A-77636, CY-208,243, SKF-89145, SKF-89626, 7,8-Dihydroxy-5-phenyl-octahydrobenzo[h]isoquinoline, Cabergoline, Pergolide, levodopa and carbidopa.

5. The method of claim 2, wherein the agonist of GHSR1αis MK0677 and the agonist of DRD1 is SKF81297.

6. The method of claim 5, wherein the MK0677 is combined with SKF81297 in a ratio of about 1:1.5.

7. The method of claim 2, wherein the agonist of DRD1 is selected based on 200-fold selectivity for D1.

8. A method for treatment of hippocampal synaptic dysfunction and Alzheimer's disease in a patient comprising the step of simultaneously applying a first drug and a second drug to promote the heteromerization of GHSR1αand DRD1, wherein the first drug is an agonist of GHSR1αand the second drug is an agonist of DRD1.

9. The method of claim 8, wherein the step of simultaneously applying a first drug and a second drug further comprises the step of combining the first drug and the second drug into a fixed-dose combination (FDC) product in a single dosage form.

10. The method of claim 8, further comprising the step of selecting the first drug from the group consisting of: Adenosine, Alexamorelin, Anamorelin, Capromorelin, CP-464709, Cortistatin-14, Examorelin (hexarelin), Ghrelin (lenomorelin), GHRP-1, GHRP-3, GHRP-4, GHRP-5, GHRP-6, Ibutamoren (MK-0677), Ipamorelin, L-692,585, LY-426410, LY-444711, Macimorelin, Pralmorelin, (GHRP-2), Relamorelin, SM-130,686, Tabimorelin and Ulimorelin.

11. The method of claim 8, further comprising the step of selecting the second drug from the group consisting of: dopamine, A-86929, Dihydrexidine, Dinapsoline, Dinoxyline, Doxanthrine, SKF-81297, SKF-82958, SKF-38393, Fenoldopam, 6-Br-APB, Stepholidine, A-68930, A-77636, CY-208,243, SKF-89145, SKF-89626, 7,8-Dihydroxy-5-phenyl-octahydrobenzo[h]isoquinoline, Cabergoline, Pergolide, levodopa and carbidopa.

12. The method of claim 8, wherein the first drug is MK0677 and the second drug is SKF81297.

13. A drug composition for rescuing hippocampal synaptic function in a patient and for rescuing cognition in Alzheimer's disease patients comprising an agonist of GHSR1 and an agonist of DRD1.

14. The drug composition of claim 13, wherein the ratio of the agonist of GHSR1αto the agonist of DRD1 is configured to promote the heteromerization of GHSR1αand DRD1.

15. The drug composition of claim 13, wherein the agonist of GHSR1 and the agonist of DRD1 are combined into a fixed-dose combination (1-DC) product in a single dosage form.

16. The drug composition of claim 13, wherein the agonist of GHSR1α is selected from the group consisting of: Adenosine, Alexamorelin, Anamorelin, Capromorelin, CP-464709, Cortistatin-14, Examorelin (hexarelin), Ghrelin (lenomorelin), GHRP-1, GHRP-3, GHRP-4, GHRP-5, GHRP-6, Ibutamoren (MK-0677), Ipamorelin, L-692,585, LY-426410, LY-444711, Macimorelin, Pralmorelin, (GHRP-2), Relamorelin, SM-130,686, Tabimorelin and Ulimorelin.

17. The drug composition of claim 13, wherein the agonist of DRD1 is selected from the group consisting of: dopamine, A-86929, Dihydrexidine, Dinapsoline, Dinoxyline, Doxanthrine, SKF-81297, SKF-82958, SKF-38393, Fenoldopam, 6-Br-APB, Stepholidine, A-68930, A-77636, CY-208,243, SKF-89145, SKF-89626, 7,8-Dihydroxy-5-phenyl-octahydrobenzo[h]isoquinoline, Cabergoline, Pergolide, levodopa and carbidopa.

18. The drug composition of claim 13, wherein the agonist of GHSR1α is MK0677 and the agonist of DRD1 is SKF81297.

19. The drug composition of claim 18, wherein the MK0677 is combined with SKF81297 in a ratio of about 1:1.5.

20. A fixed dose combination drug for rescuing hippocampal synaptic function in a patient and for rescuing cognition in Alzheimer's disease patients comprising an agonist of GHSR1 and an agonist of DRD1 wherein the ratio of the agonist of GHSR1α to the agonist of DRD1 is configured to promote the heteromerization of GHSR1α and DRD1.
